SEQUENCE LISTING (12 sequences)

SEQ ID NO: 1 - DNA, 18 bp, synthetic construct
  source 1..18: mol_type = other DNA
taccacttcacaagtcgg

SEQ ID NO: 2 - DNA, 23 bp, synthetic construct
  source 1..23: mol_type = other DNA
ctgcaagtgcatcatcgttgttc

SEQ ID NO: 3 - DNA, 20 bp, synthetic construct
  source 1..20: mol_type = other DNA
tgaccccaagaaggaatggg

SEQ ID NO: 4 - DNA, 20 bp, synthetic construct
  source 1..20: mol_type = other DNA
accttagggcagatgcagtt

SEQ ID NO: 5 - DNA, 22 bp, synthetic construct
  source 1..22: mol_type = other DNA
catccacagcttgagtgtgacg

SEQ ID NO: 6 - DNA, 22 bp, synthetic construct
  source 1..22: mol_type = other DNA
ggcttcagggtcaaggcaaact

SEQ ID NO: 7 - DNA, 22 bp, synthetic construct
  source 1..22: mol_type = other DNA
ccgctggcatttctgttgctgt

SEQ ID NO: 8 - DNA, 23 bp, synthetic construct
  source 1..23: mol_type = other DNA
cagggatcacctccaaattgacg

SEQ ID NO: 9 - DNA, 20 bp, synthetic construct
  source 1..20: mol_type = other DNA
ccaagtgctgccgtcatttt

SEQ ID NO: 10 - DNA, 20 bp, synthetic construct
  source 1..20: mol_type = other DNA
ctcaacacgtgggcaggata

SEQ ID NO: 11 - DNA, 21 bp, synthetic construct
  source 1..21: mol_type = other DNA
aagatggtgatgggcttcccg

SEQ ID NO: 12 - DNA, 22 bp, synthetic construct
  source 1..22: mol_type = other DNA
tggcaaagtggagattgttgcc

(19) United States
(12) Patent Application Publication
(10) Pub. No.: US 2026/0167648 A1
(43) Pub. Date: Jun. 18, 2026

(54) NOVEL DECURSIN DERIVATIVES AND ANTI-INFLAMMATORY COMPOSITION COMPRISING THE SAME

(71) Applicant: The Industry & Academic Cooperation in Chungnam National University (IAC), Daejeon (KR)

(72) Inventors: Eun-Kyeong JO, Daejeon (KR); Bokeum JUNG, Daejeon (KR); Eunwoo GWAK, Hwaseong-si (KR); Gyu Yong SONG, Daejeon (KR); Seok Woo LEE, Daejeon (KR); Jong-Beom HEO, Daejeon (KR); Yuri SEO, Daejeon (KR); Bomi LEE, Daejeon (KR)

(73) Assignee: The Industry & Academic Cooperation in Chungnam National University (IAC), Daejeon (KR)

(21) Appl. No.: 19/409,184

(22) Filed: Dec. 4, 2025

(30) Foreign Application Priority Data
Dec. 12, 2024 (KR) 10-2024-0184627

(51) Int. Cl. C07D 493/04; A61K 8/49; A61K 31/37; A61K 31/381; A61K 31/4184; A61K 31/4433; A61K 31/453; A61K 31/496; A61K 31/5377; A61P 29/00; A61Q 19/00

(52) U.S. Cl. CPC C07D 493/04; A61K 8/498; A61K 31/37; A61K 31/381; A61K 31/4184; A61K 31/4433; A61K 31/453; A61K 31/496; A61K 31/5377; A61P 29/00; A61Q 19/00

(57) ABSTRACT

Disclosed are novel decursin derivatives, methods for preparing the same, and anti-inflammatory compositions comprising the same as active ingredients. The decursin derivative is represented by Chemical Formula:

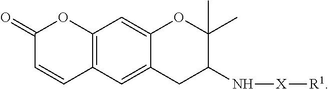

X represents —C(═O)—, —C(═O)NH—, or a direct bond; R¹ is —(CH═CH)—R², —(CH₂)ₙ—R³, or —(CH₂CH₂O)ₘ—R⁴; n is an integer of 0 to 8, and m is an integer of 1 to 3; R², R³, and R⁴ are each independently CH₃, C3-C6 cycloalkyl, 5- to 6-membered heterocycloalkyl, C6-C12 aryl, C4-C12 heteroaryl, 1,4-naphthoquinonyl, —CH═CH—R⁵, —S—R⁶, or —S(═O)—R⁷; R⁵, R⁶, and R⁷ are each independently C6-C12 aryl, C4-C12 heteroaryl, or 1,4-naphthoquinonyl; and R², R³, R⁴, R⁵, R⁶, and R⁷ may each independently be unsubstituted or substituted with a substituent.

### CROSS-REFERENCE TO PRIOR APPLICATION

This application claims priority to Korean Patent Application No. 10-2024-0184627 (filed on Dec. 12, 2024), which is all hereby incorporated by reference in its entirety.

### SEQUENCE LISTING

This application contains a Sequence Listing submitted via XML file and hereby incorporated by reference in its entirety. The sequence listing is named “OP25-1241US”, created on Dec. 1, 2025 and 11,725 bytes in size.

### BACKGROUND

The present invention relates to novel decursin derivatives, methods for preparing the same, and anti-inflammatory compositions comprising the same as active ingredients.

Decursin is one of the bioactive compounds contained in Angelica gigas, a traditional herbal medicine used in Korea and China. According to various studies, decursin inhibits the growth of diverse cancer cells, induces apoptosis, and promotes the proliferation of human immune cells such as B cells, T cells, and natural killer (NK) cells, thereby exhibiting antitumor immune activity. Its effect of inhibiting angiogenesis can be utilized in cancer therapy. Furthermore, by enhancing the activity of nerve growth factor (NGF), decursin promotes neuronal growth and protects nerve cells from oxidative stress, thereby having potential utility in the treatment of neurodegenerative diseases. In addition, by inhibiting NF-κB in macrophages, which mediate inflammatory responses, it suppresses the production of pro-inflammatory cytokines such as IL-6, IL-8, MCP-1, and TNF-α, thereby exhibiting anti-inflammatory activity. Due to such diverse bioactivities, decursin has attracted attention in the medical and pharmaceutical fields; however, for the development of decursin-based drugs, it is necessary to improve its low oral bioavailability and to develop derivatives with enhanced potency.

Decursin has the following structure based on a dihydropyranocoumarin skeleton.

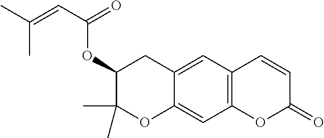 [SMILES: CC(C)=CC(=O)O[C@H]1Cc2cc3ccc(=O)oc3cc2OC1(C)C]

As predicted from its structure, decursin is a lipophilic compound that is almost insoluble in water, and improvement in solubility is essential to enhance its bioavailability. Poorly soluble hydrophobic compounds cannot be easily excreted from the body and tend to accumulate in lipid layers, raising concerns of chronic toxicity. The methyl-butenoyl ester bond of decursin is easily hydrolyzed by esterases in the body, and the double bond in the methyl-butenoyl side chain is susceptible to oxidation; thus, it can be readily oxidized by hepatic cytochrome P450 enzymes. Therefore, decursin introduced into the body undergoes rapid metabolism, making it difficult to maintain its blood concentration. Decursinol, generated by hydrolysis, has relatively low permeability through cell membranes or the blood-brain barrier, making it difficult for the drug to enter target cells, thereby reducing the overall bioavailability of the drug.

Therefore, to improve the bioavailability of decursin, strategies may be considered in which the ester bond is replaced with a more stable bond or steric hindrance around the ester bond is increased to hinder esterase access, and the double bond is removed or modified to prevent oxidation.

Korean Patent No. 10-0837733 discloses various derivatives in which the ester bond is replaced with an oxime bond, and Korean Patent No. 10-1413793 attempts to solve the above problem by introducing a heteroatom into the hydropyran skeleton of decursin. Replacing the ester bond with an amide bond is one useful strategy to improve solubility and suppress hydrolysis. Korean Patent No. 10-0966027 discloses a decursin derivative in which the ester bond of decursin is replaced with an amide bond and a method for preparing the same. However, since the method introduced an amino group by introducing and reducing a nitro group at the α-position of the carbonyl group using 8,8-dimethyl-7,8-dihydropyrano[3,2-g]chromene-2,6-dione as a starting material, deoxygenation was required to construct the decursin skeleton, making it impossible to apply the method to the preparation of diverse decursin amide derivatives. Due to the difficulty in preparing decursin amine in which the hydroxyl group of decursinol is substituted with an amino group, despite the recognition that introduction of an amide bond could improve drug properties, development of derivatives based on decursin amine has not been achieved.

### SUMMARY

An object of the present invention is to provide novel decursin derivatives that can improve the aqueous solubility of decursin and enhance bioavailability by not including substituents susceptible to oxidation, and methods for preparing the same.

Another object of the present invention is to provide a composition for the prevention, treatment, or improvement of symptoms of inflammatory diseases, comprising the decursin derivatives as active ingredients.

The technical problems to be solved by the present invention, even if not explicitly mentioned above, will be clearly understood by those skilled in the art.

### Technical Solution

To achieve the above-described objects, the present invention relates to decursin derivatives represented by Chemical Formula 1 below.

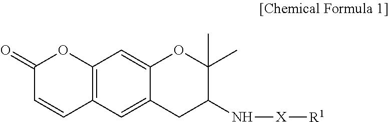

In Chemical Formula 1, X represents —C(═O)—, —C(═O)NH—, or a direct bond;
R¹ is —(CH═CH)—R², —(CH₂)ₙ—R³, or —(CH₂CH₂O)ₘ—R⁴;
n is an integer of 0 to 8, and m is an integer of 1 to 3;
R², R³, and R⁴ are each independently CH₃, C3-C6 cycloalkyl, 5- to 6-membered heterocycloalkyl, C6-C12 aryl, C4-C12 heteroaryl, 1,4-naphthoquinonyl, —CH═CH—R⁵, —S—R⁶, or —S(═O)—R⁷;
R⁵, R⁶, and R⁷ are each independently C6-C12 aryl, C4-C12 heteroaryl, or 1,4-naphthoquinonyl; and
R², R³, R⁴, R⁵, R⁶, and R⁷ may each independently be unsubstituted or substituted with a substituent.

Hereinafter, unless otherwise specified, the definitions of X, n, m, and R¹ to R⁷ shall be as defined above.

The term “alkyl” refers to a straight-chain or branched saturated hydrocarbon; the term “aryl” refers to an aromatic hydrocarbon ring; and the term “heteroaryl” refers to an aromatic ring containing one or more heteroatoms selected from nitrogen, oxygen, and sulfur.

The substituent may be selected from the group consisting of —F, —Cl, —Br, —CF₃, —OMe, —NO₂, -Me, —OH, —OAc, —SH, and —N(CH₃)₂, or a combination thereof, and one or more substituents may be present. The 5- to 6-membered heterocycloalkyl may be, for example, pyrrolidine, piperidine, morpholine, thiomorpholine, or piperazine; and the heteroaryl may be, for example, pyridine, pyrazine, furan, thiophene, or benzimidazole.

Hereinafter, numerical ranges described in the present specification shall include both endpoints unless otherwise specified. Throughout the present specification, when a component is described as “comprising,” this means that other components are not excluded unless expressly stated otherwise, and that one or more additional components may further be included. In this specification, “/” and “and/or” are to be construed as describing each of two specific features or elements either alone or in combination with the other. For example, “A/B” and “A and/or B” shall each be interpreted to mean (i) A, (ii) B, or (iii) both A and B, each independently. Hereinafter, in describing the invention, detailed descriptions of known techniques related to the invention may be omitted when it is determined that such descriptions would unnecessarily obscure the gist of the invention.

The decursin derivatives of the present invention can be classified into amide derivatives, urea derivatives, and amine derivatives depending on X. These derivatives are all decursin derivatives wherein the ester group of decursin, which is readily hydrolyzed in vivo, is replaced, thereby making hydrolysis more difficult while improving solubility, enhancing bioavailability, and preventing accumulation in lipid layers in the body, thus alleviating concerns regarding long-term toxicity. In addition, by replacing the methyl-butenoyl ester bond with an aromatic, heteroaromatic, or alkyl group, the problem of decursin being susceptible to oxidation can be alleviated.

Representative examples of the compounds of Chemical Formula 1 include the following compounds; however, the scope of the present invention is not limited thereto.

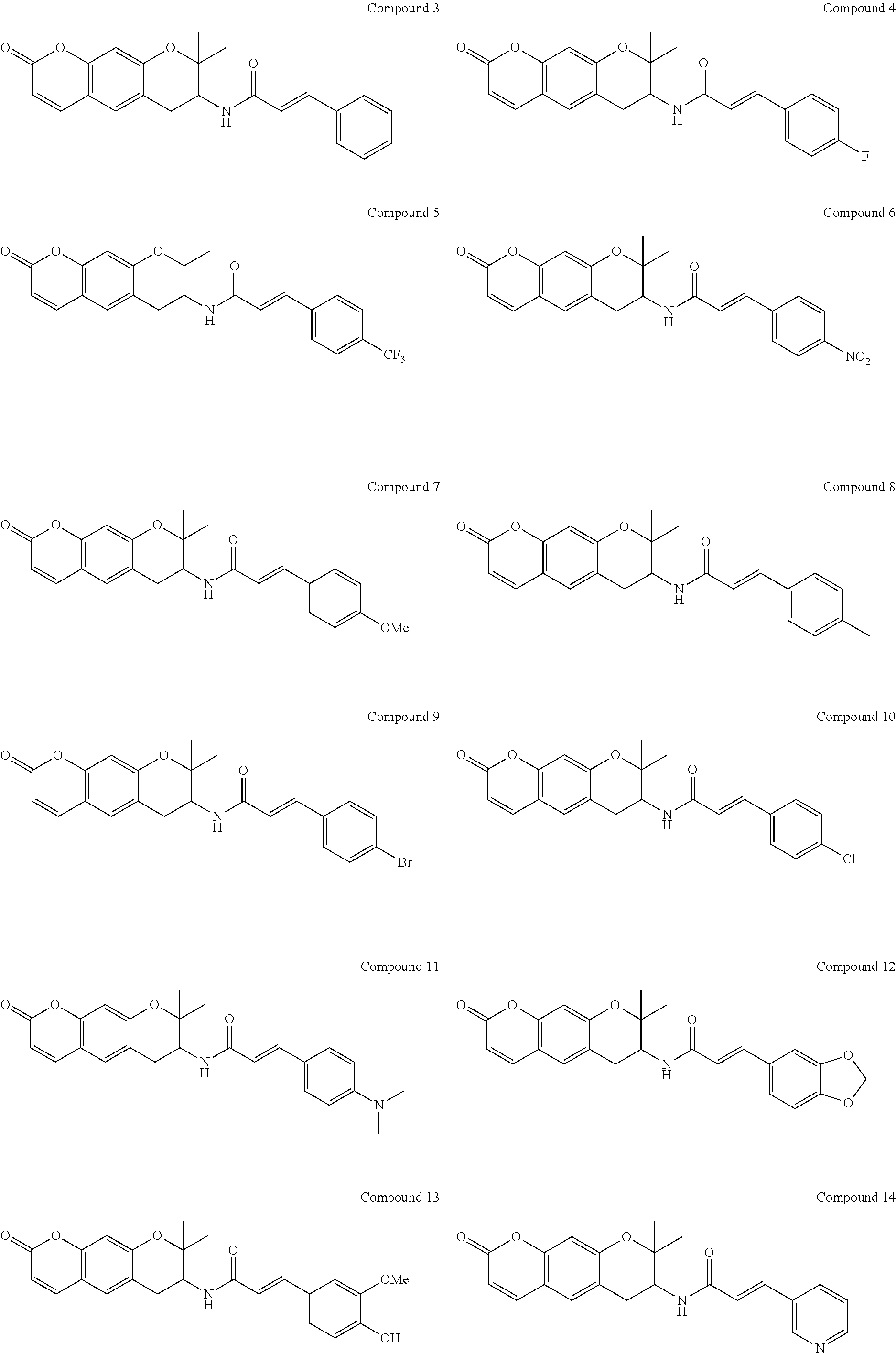

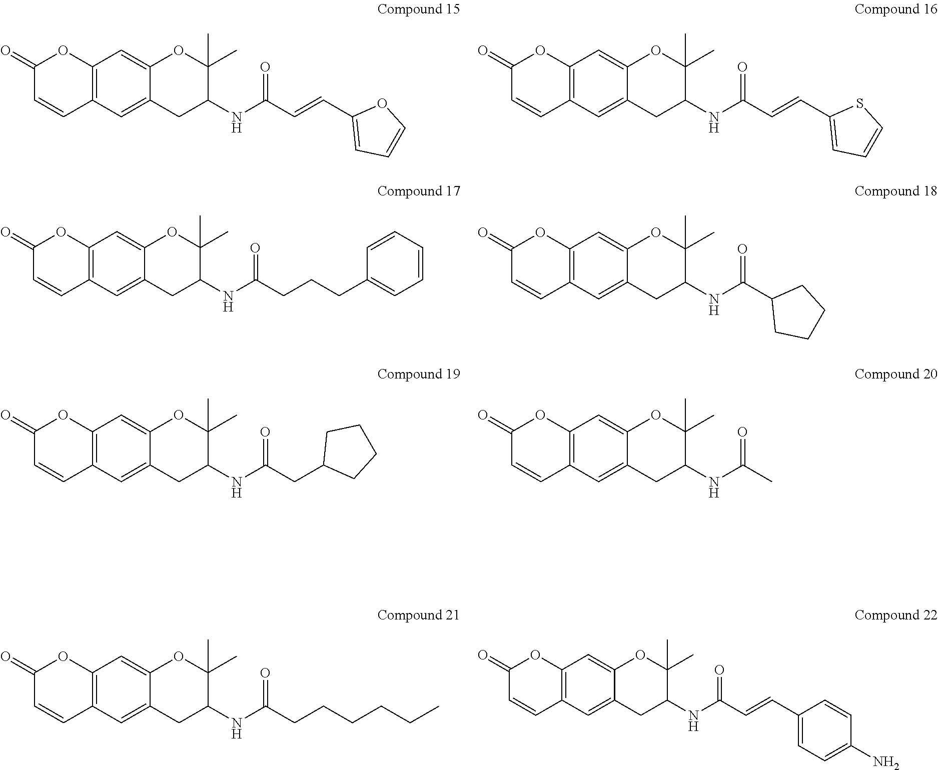

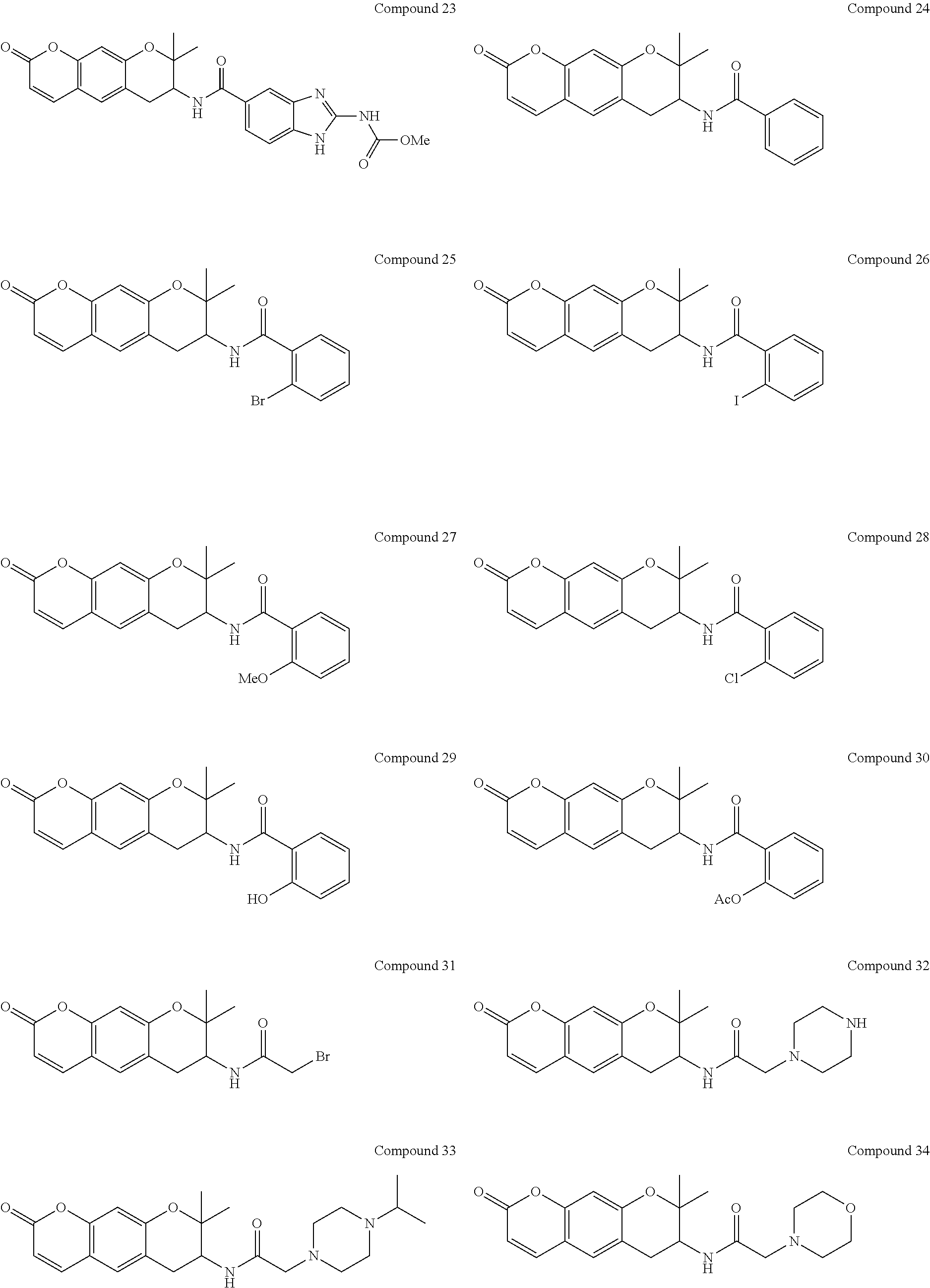

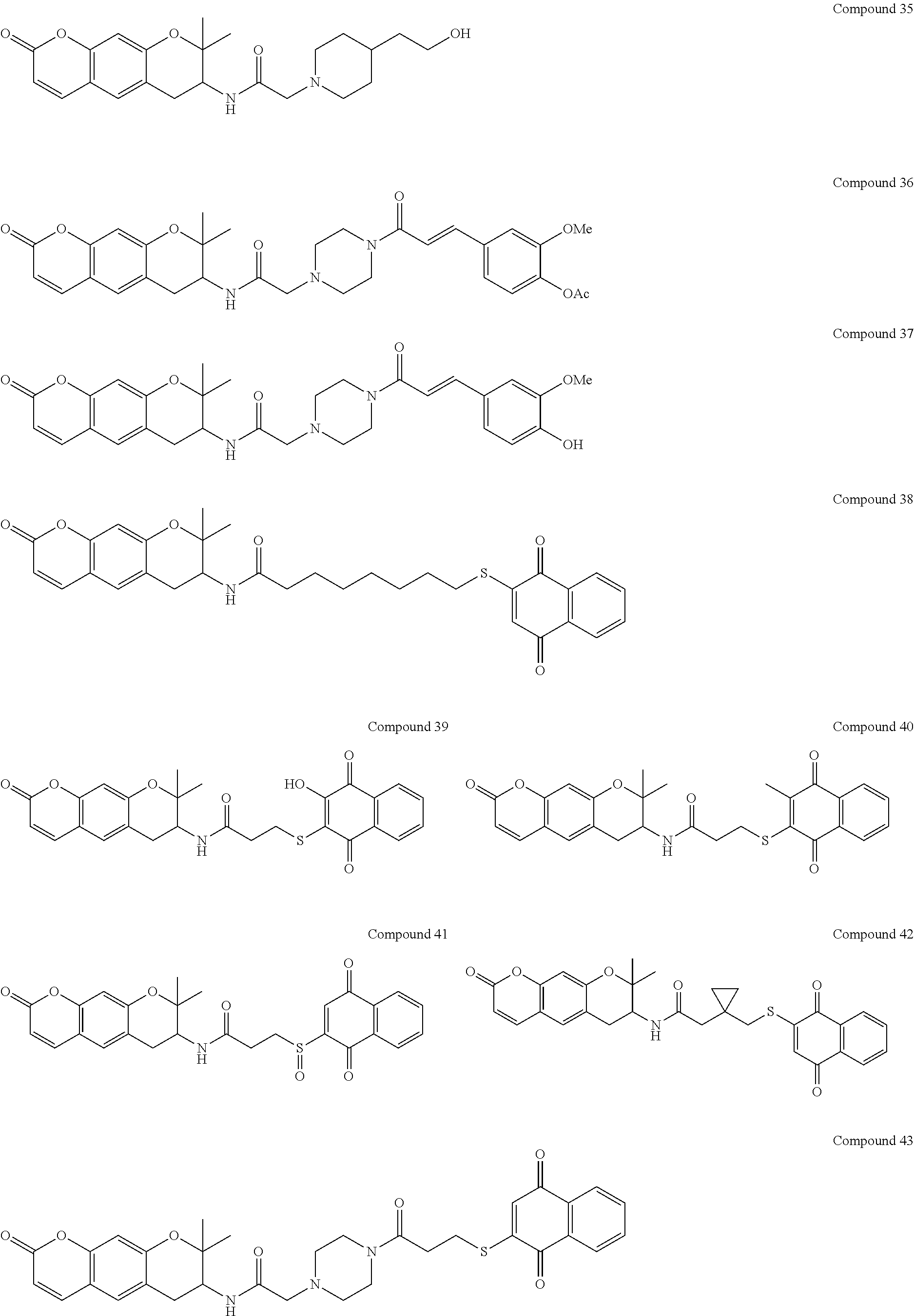

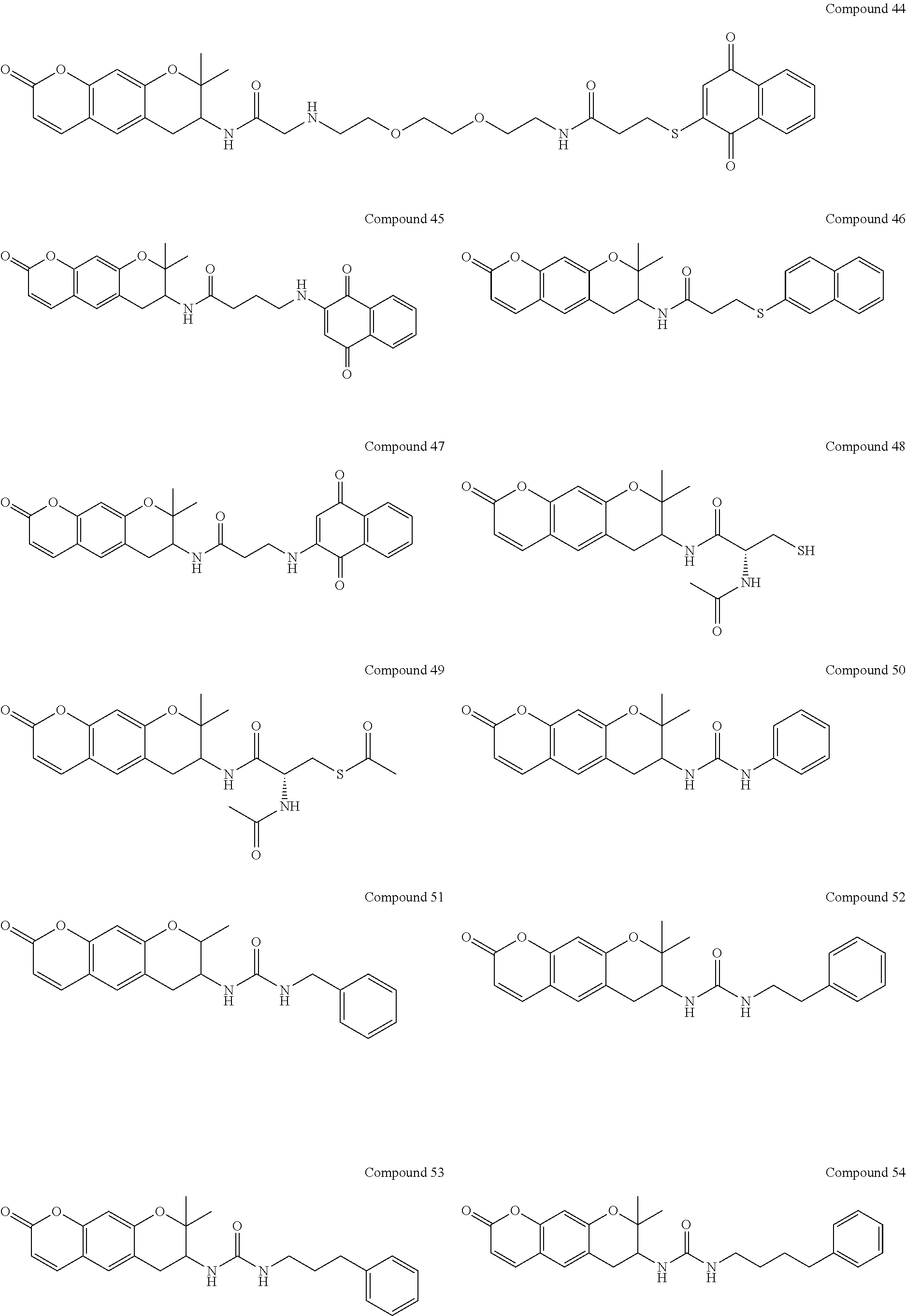

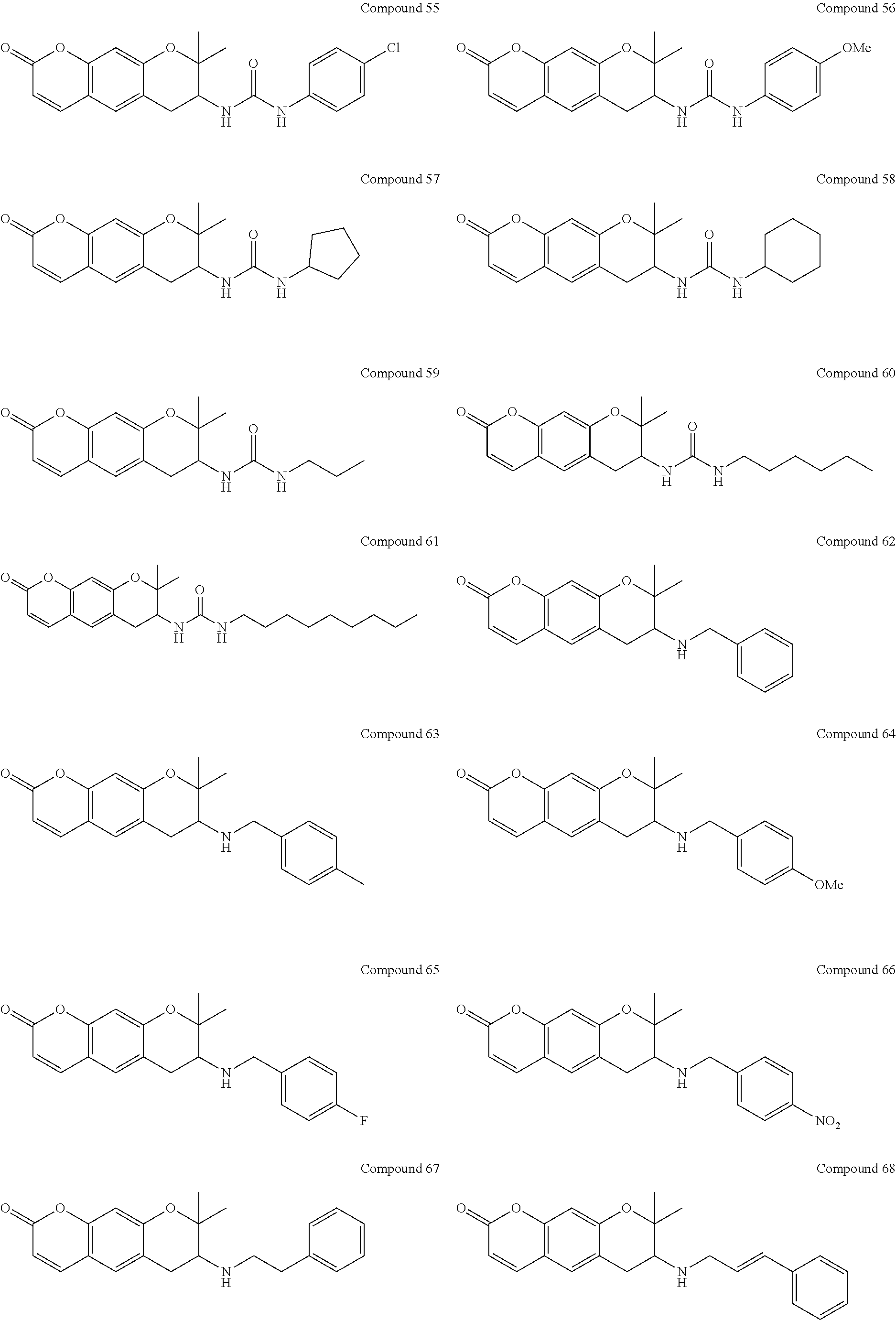

The compounds of Chemical Formula 1 may exist not only in free form but also in the form of pharmaceutically acceptable salts. Acid addition salts formed by pharmaceutically acceptable free acids are useful. Such acid addition salts may be prepared by conventional methods, for example by dissolving the compound in an aqueous solution containing an excess of acid and precipitating the resulting salt using a water-miscible organic solvent such as methanol, ethanol, acetone, or acetonitrile. Alternatively, the compound and an equivalent amount of an acid are heated in water or in an alcohol (e.g., glycol monomethyl ether), and the resulting mixture is then evaporated to dryness, or the precipitated salt may be collected by suction filtration.

The free acid may be either an organic acid or an inorganic acid. Examples of inorganic acids include hydrochloric acid, phosphoric acid, sulfuric acid, nitric acid, carbonic acid, hydroiodic acid, and stannic acid. Examples of organic acids include methanesulfonic acid, p-toluenesulfonic acid, acetic acid, trifluoroacetic acid, citric acid, maleic acid, succinic acid, oxalic acid, benzoic acid, tartaric acid, fumaric acid, mandelic acid, propionic acid, lactic acid, glycolic acid, gluconic acid, galacturonic acid, glutamic acid, glutaric acid, glucuronic acid, aspartic acid, ascorbic acid, and vanillic acid.

When the compounds of the present invention are in the form of salts, their aqueous solubility can be further increased and their stability and absorption can be improved, thereby providing improved bioavailability.

Another aspect of the present invention relates to methods for preparing decursin derivatives of Chemical Formula 1. The compounds of Chemical Formula 1 can be prepared from decursin amine (Compound 1). Decursin amine can in turn be obtained from decursinol (I) as a starting material, for example, according to the reaction scheme below.

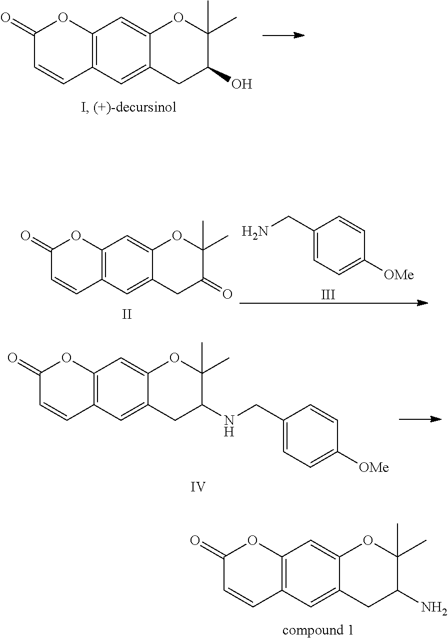

The present invention relates to methods for preparing decursin derivatives of Chemical Formula 1 using decursin amine or Compound II, an intermediate of decursin amine, as starting materials. Accordingly, detailed descriptions of methods for preparing decursin amine or its intermediates will be omitted. A method for preparing decursin amine has been filed as Korean Patent Application No. 10-2024-0105490 on Aug. 7, 2024, the disclosure of which is incorporated herein by reference in its entirety.

The methods for preparing the compounds of Chemical Formula 1, categorized by type, are as follows.

### (1) Amide Derivatives

Among the compounds of Chemical Formula 1, those wherein X is —C(═O)— fall within this category.

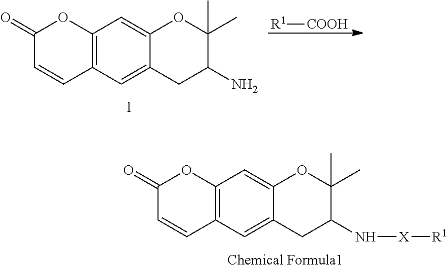

Compounds in this category can be prepared by reacting decursin amine (1) with R¹—COOH, i.e., carboxylic acids having the structure R²—(CH═CH)—COOH, R³—(CH₂)ₙ—COOH or R⁴—(CH₂CH₂O)ₘ—COOH, or derivatives thereof. Suitable derivatives of the carboxylic acids include acyl halides, esters, and the carboxylic acids themselves. In this step, bases conventionally used as catalysts in reactions between amines and carboxylic acid derivatives may be employed. Examples of such bases include triethylamine (TEA), N,N-dimethylaminopyridine, and 4-methylmorpholine, but the present invention is not limited thereto. The formation of amides by reactions between carboxylic acids or carboxylic acid derivatives and amines is well known and widely used in the field of organic synthesis. Since such known techniques can also be employed in the present invention, detailed descriptions of each specific case are omitted.

### (2) Urea Derivatives

Among the compounds of Chemical Formula 1, those wherein X is —C(═O)NH— fall within this category.

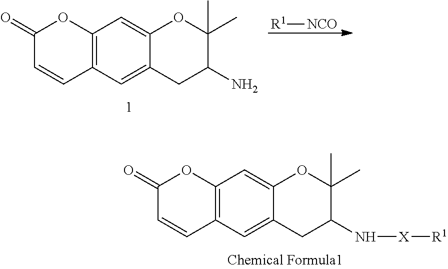

Compounds in this category can be prepared by reacting decursin amine (1) with R¹—NCO, i.e., isocyanates having the structure R²—(CH═CH)—NCO, R³—(CH₂)ₙ—NCO or R⁴—(CH₂CH₂O)ₘ—NCO, or derivatives thereof. The preparation of urea compounds by reactions between amines and isocyanates is well known and widely used in the field of organic synthesis. Since such known techniques can also be employed in the present invention, detailed descriptions of each specific case are omitted.

### (3) Amine Derivatives

Among the compounds of Chemical Formula 1, those wherein X is a direct bond fall within this category.

Compounds in this category can be prepared by reducing imines or enamines formed by a reaction between an amino group (—NH₂) and a carbonyl group (—C(═O)—).

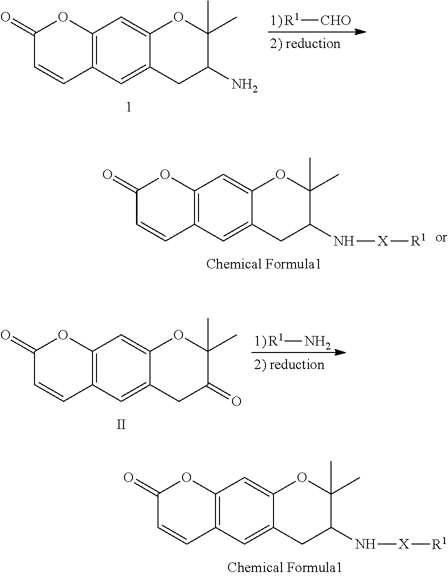

That is, the method comprises: (A) reacting decursin amine (1) with R¹—CHO, i.e., aldehydes having the structures R²—(CH═CH)—CHO, R³—(CH₂)ₙ—CHO or R⁴—(CH₂CH₂O)ₘ—CHO, or alternatively coupling compound (II) with R¹—NH₂, i.e., amines having the structures R²—(CH═CH)—NH₂, R³—(CH₂)ₙ—NH₂ or R⁴—(CH₂CH₂O)ₘ—NH₂, to produce imine or enamine intermediates; and

### (B) Reducing the Imine or Enamine Intermediates to the Corresponding Amines.

The step of reducing the imine or enamine intermediates to the corresponding amines is preferably carried out under mild conditions, because the imines or enamines must be selectively reduced without affecting the aromatic ring or double bond of the coumarin ring or the structures of R³ and R⁴. For example, sodium borohydride, sodium cyanoborohydride, or sodium triacetoxyborohydride may be employed, but the present invention is not limited thereto. It will be readily apparent to those skilled in the art that an appropriate reducing agent can be selected and used in view of the structure of each compound.

The present invention also provides an anti-inflammatory pharmaceutical composition comprising, as an active ingredient, a decursin derivative represented by Chemical Formula 1 or a pharmaceutically acceptable salt thereof. The anti-inflammatory pharmaceutical composition of the present invention can be used for the prevention or treatment of inflammatory diseases.

The composition of the present invention exhibited anti-inflammatory effects in vitro by effectively suppressing the expression of the pro-inflammatory cytokine IL-6 and the chemokines Ccl2, Cxcl2, Cxcl5, and Cxcl10, as compared with conventional decursin or decursinol. Furthermore, because the composition does not contain structural units that cause hydrolysis or oxidation in vivo and the introduction of an amino group increases solubility, the bioavailability of the composition is improved relative to decursin or decursinol. Accordingly, the composition is expected to have even greater utility.

The inflammatory diseases may include gastritis, stomatitis, atopic dermatitis, acne, psoriasis, sinusitis, rhinitis, conjunctivitis, asthma, dermatitis, inflammatory collagen vascular disease, glomerulonephritis, encephalitis, inflammatory enteritis, chronic obstructive pulmonary disease, sepsis, septic shock, pulmonary fibrosis, undifferentiated spondyloarthropathy, undifferentiated arthropathy, arthritis, inflammatory osteolysis, chronic inflammatory diseases caused by viral or bacterial infections, ulcerative colitis, inflammatory bowel disease, rheumatoid arthritis, reactive arthritis, osteoarthritis, scleroderma, osteoporosis, atherosclerosis, myocarditis, endocarditis, pericarditis, cystic fibrosis, Hashimoto's thyroiditis, Graves' disease, leprosy, syphilis, Lyme disease, borreliosis, neuroborreliosis, tuberculosis, sarcoidosis, lupus, chilblain lupus, tuberculous lupus, lupus nephritis, systemic lupus erythematosus, macular degeneration, uveitis, irritable bowel syndrome, Crohn's disease, Sjögren's syndrome, fibromyalgia, chronic fatigue syndrome, chronic fatigue immune deficiency syndrome, myalgic encephalomyelitis, amyotrophic lateral sclerosis, Parkinson's disease, and multiple sclerosis, and preferably may be inflammatory diseases associated with the expression of IL-6 and/or chemokines.

The anti-inflammatory pharmaceutical composition according to the present invention may be prepared, by methods well known in the pharmaceutical field, as dosage forms such as powders, granules, tablets, capsules, or injectable preparations, either as such or in combination with pharmaceutically acceptable carriers, excipients, diluents, and the like.

The pharmaceutical composition according to the present invention may be administered in a pharmaceutically effective amount. In the present invention, the term “pharmaceutically effective amount” refers to an amount sufficient to treat a disease with a reasonable benefit-to-risk ratio applicable to medical treatment. The effective dosage level may be determined depending on factors such as the type and severity of the disease, the age and sex of the patient, the activity of the drug, the patient's sensitivity to the drug, the time and route of administration, the rate of excretion, the duration of treatment, concomitant medications, and other factors well known in the medical field. Taking all of the above factors into account, it is important to administer an amount that achieves maximum efficacy with a minimal dosage and without side effects, which can be readily determined by those skilled in the art. Generally, for adults, the compound may be administered at a daily dosage of 0.1 to 100 mg per kg of body weight, in one or several divided doses.

The composition of the present invention may be administered alone or in combination with other therapeutic agents, and may be administered sequentially or concurrently with conventional therapeutic agents.

Another aspect of the present invention relates to a health functional food composition for the prevention or alleviation of inflammatory symptoms, comprising, as an active ingredient, a decursin derivative represented by Chemical Formula 1 or a salt thereof.

The effective dosage of the health functional food composition of the present invention may be set in accordance with the effective dosage of the above-described pharmaceutical composition; however, in cases of long-term intake for purposes of health maintenance or hygiene, the dosage may be below that range. The health functional food of the present invention may be formulated in the form of tablets, capsules, pills, or liquids, and examples of foods to which the composition of the present invention can be added include various food products, beverages, chewing gum, tea, confectionery, alcoholic drinks, chocolate, multivitamin preparations, and health functional food products.

The health functional food of the present invention may be used not only in humans but also in animals. In such cases, the health functional food may be employed as a feed additive composition for the prevention or alleviation of inflammatory symptoms. The animals include mammals, and examples of mammals to which the composition may be applied include companion animals such as dogs and cats, livestock such as cattle, pigs, and sheep, and laboratory rodents; however, the present invention is not limited thereto.

Another aspect of the present invention relates to a cosmetic composition for the prevention or alleviation of inflammatory symptoms, comprising, as an active ingredient, a decursin derivative compound represented by Chemical Formula 1 or a salt thereof. The composition of the present invention may be usefully applied, for example, to the prevention or alleviation of skin inflammation such as atopic dermatitis or acne. The composition may be used together with other additives widely employed in the cosmetic field and may be formulated into cosmetic preparations such as skin, skin lotions, skin softeners, skin toners, astringents, lotions, nourishing lotions, milk lotions, moisture lotions, nourishing creams, massage creams, moisture creams, hand creams, essences, nourishing essences, packs, soaps, cleansing foams, cleansing lotions, cleansing creams, body cleansers, body lotions, shampoos, emulsions, foundations, pressed powders, loose powders, and eye shadows.

As described above, the decursin derivative compounds of the present invention do not contain oxidation-sensitive substituents or hydrolysis-susceptible ester groups present in decursin and, because they include functional groups derived from amino groups, they exhibit excellent solubility in water. Accordingly, they can overcome the problem of low bioavailability observed in conventional decursin derivatives.

In addition, in vitro assays show that the compounds exhibit superior anti-inflammatory activity compared with decursin and decursinol, and are therefore useful as active ingredients in various compositions for the prevention, treatment, or alleviation of inflammatory symptoms.

### BRIEF DESCRIPTION OF THE DRAWINGS

FIG. 1 shows graphs illustrating the cytotoxicity of decursin derivatives according to the prior art and representative decursin derivatives of the present invention.

FIG. 2 shows graphs illustrating the anti-inflammatory activity of decursin derivatives according to the prior art.

FIG. 3 shows graphs illustrating the anti-inflammatory activity of amide-type decursin derivatives according to the present invention.

FIG. 4 shows graphs illustrating the anti-inflammatory activity of urea-type decursin derivatives according to the present invention.

FIG. 5 shows graphs illustrating the anti-inflammatory activity of amine-type decursin derivatives according to the present invention.

FIGS. 6 and 7 show graphs illustrating the effects of the decursin derivatives of the present invention on the mRNA expression of pro-inflammatory cytokines and chemokines in macrophages.

### DETAILED DESCRIPTION

The present invention will now be described in further detail with reference to the accompanying drawings and embodiments. However, such drawings and embodiments are merely illustrative for facilitating the understanding of the technical concept and scope of the present invention, and the technical scope of the present invention is not limited or altered thereby. It will be apparent to those skilled in the art that various modifications and alterations can be made within the scope of the technical concept of the present invention based on these examples.

### EXAMPLES

### Preparation Example 1: Preparation of Decursin Amine (Compound 1)

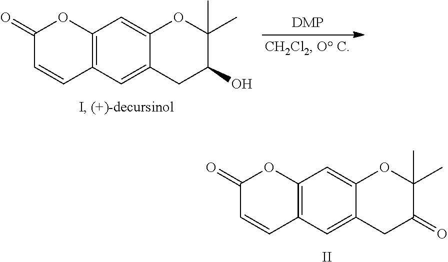

### 1) Preparation of 8,8-dimethyl-8-hydro-2H,6H-pyrano[3,2-g]chromene-2,7-dione (Compound II)

Starting material I (5,000 mg, 20.30 mmol) was dissolved in dichloromethane, and the solution was cooled at 0° C. for 20 minutes. After 20 minutes, Dess-Martin periodinane (DMP; 12,915 mg, 30.45 mmol) was added, and the reaction mixture was stirred for 4 hours and 30 minutes. Upon completion, the reaction mixture was quenched with saturated sodium bicarbonate solution and extracted with dichloromethane. The dichloromethane extract was dried over anhydrous sodium sulfate, concentrated, and the residue was purified by silica gel column chromatography using dichloromethane to afford Compound II as a white solid (4862 mg, 98%).

Melting point (mp): 163.1° C.

¹H NMR (400 MHz, CDCl₃): δ 7.64 (d, J=9.5 Hz, 1H), 7.22 (s, 1H), 6.97 (s, 1H), 6.31 (d, J=9.5 Hz, 1H), 3.66 (s, 2H), 1.45 (s, 6H).

¹³C NMR (101 MHz, CDCl₃): δ 208.19, 160.74, 156.08, 154.66, 142.86, 127.10, 119.33, 114.60, 114.55, 106.79, 83.31, 38.73, 24.11.

MS (ESI): m/z [M+H]⁺ 245.1, found.

### 2) Preparation of 7-(4-methoxybenzyl)amino-8,8-dimethyl-7,8-dihydro-2H,6H-pyrano[3,2-g]chromen-2-one (Compound IV)

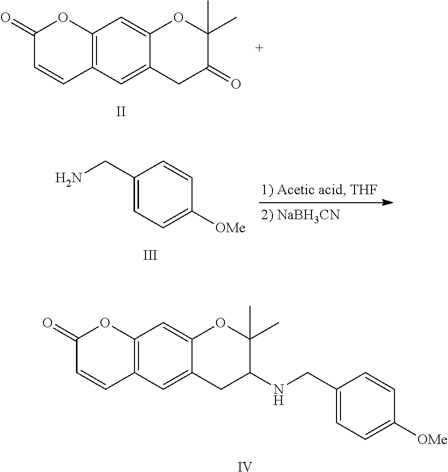

Compound II (1,000 mg, 4.09 mmol), prepared as described in 1) above, was dissolved in tetrahydrofuran. Acetic acid (266 μL) and 4-methoxybenzylamine (802 μL, 6.14 mmol) were added, and the mixture was stirred at room temperature for 15 minutes. Sodium cyanoborohydride (514 mg, 8.18 mmol) was then added, and the reaction mixture was stirred at room temperature for 18 hours. After 18 hours, tetrahydrofuran was removed under reduced pressure, water was added, and the mixture was extracted with ethyl acetate. The ethyl acetate extract was dried over anhydrous sodium sulfate and concentrated. The residue was purified by silica gel column chromatography using a hexane/ethyl acetate mixture (v/v, 2:1) to afford Compound IV as a white solid (1,211 mg, 81%).

Mp: 103° C.

¹H NMR (400 MHz, CDCl₃): δ 7.59 (d, J=9.5 Hz, 1H), 7.28 (s, 1H), 7.26 (s, 1H), 7.17 (s, 1H), 6.88 (d, J=8.5 Hz, 2H), 6.76 (s, 1H), 6.22 (d, J=9.4 Hz, 1H), 3.93 (d, J=13.1 Hz, 1H), 3.82 (s, 3H), 3.77 (d, J=13.1 Hz, 1H), 3.04 (dd, J=16.0, 4.6 Hz, 1H), 2.81 (dd, J=7.9, 4.9 Hz, 1H), 2.71 (dd, J=16.0, 8.0 Hz, 1H), 1.42 (s, 3H), 1.34 (s, 3H).

¹³C NMR (101 MHz, CDCl₃): δ 161.40, 158.78, 157.01, 154.17, 143.20, 132.38, 129.20, 128.72, 117.74, 113.84, 113.01, 112.56, 104.59, 79.20, 56.18, 55.29, 51.33, 28.10, 26.51, 21.54.

MS (ESI): m/z [M+H]⁺ 366.1, found.

### 3) Preparation of 7-amino-8,8-dimethyl-7,8-dihydro-2H,6H-pyrano[3,2-g]chromen-2-one (Compound 1)

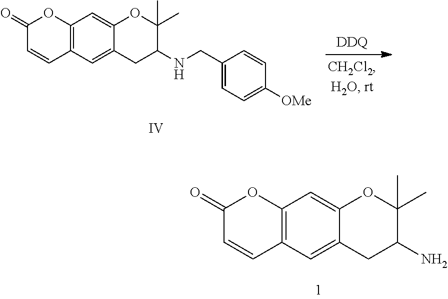

Compound IV (1,000 mg, 2.74 mmol) was dissolved in a mixture of dichloromethane and distilled water, and 2,3-dichloro-5,6-dicyano-1,4-benzoquinone (933 mg, 4.11 mmol) was added. The mixture was stirred at room temperature for 3 hours. Upon completion of the reaction, the reaction mixture was quenched with saturated aqueous sodium bicarbonate solution and extracted with dichloromethane. The dichloromethane extract was dried over anhydrous sodium sulfate, concentrated, and the residue was purified by silica gel column chromatography using a dichloromethane/methanol mixture (v/v, 30:1) to afford Compound 1 as a yellow semi-solid (470 mg, 70%).

¹H NMR (400 MHz, CDCl₃): δ 7.58 (d, J=9.5 Hz, 1H), 7.16 (s, 1H), 6.76 (s, 1H), 6.21 (d, J=9.5 Hz, 1H), 3.10-3.00 (m, 2H), 2.68-2.58 (m, 1H), 1.40 (s, 3H), 1.30 (s, 3H).

¹³C NMR (101 MHz, CDCl₃): δ 161.42, 156.82, 154.13, 143.25, 128.66, 117.62, 113.06, 112.65, 104.57, 78.97, 51.29, 31.30, 25.98, 20.90.

MS (ESI): m/z [M+H]⁺ 246.1, found.

### Preparation Example 2: Preparation of Decursin Amide Derivatives (VI)

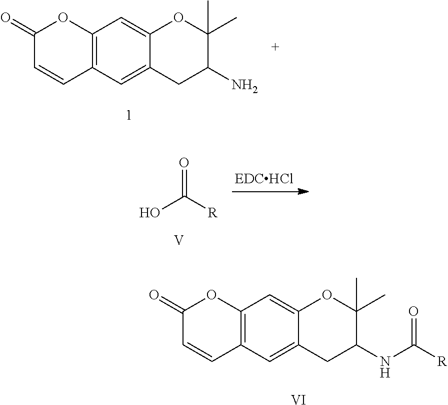

Amide derivatives of Formula (VI) were prepared by reacting Compound 1, prepared in Preparation Example 1, with carboxylic acids of Formula (V). A general reaction procedure for the amide derivatives is as follows.

Compound 1 (1.0 equiv), carboxylic acid (V) (1.2 equiv), 1-ethyl-3-(3-dimethylaminopropyl)carbodiimide hydrochloride (EDC·HCl) (1.2 equiv), triethylamine (TEA) (1.5 equiv), and 4-dimethylaminopyridine (4-DMAP) (0.6 equiv) were dissolved in dichloromethane and stirred at room temperature for 16 hours. Upon completion of the reaction, a saturated ammonium chloride solution was added to quench the reaction, and the mixture was extracted with dichloromethane. The dichloromethane extract was dried over anhydrous sodium sulfate and concentrated, and the residue was purified by silica gel column chromatography using a mixture of dichloromethane and methanol to afford decursin amide derivatives as solids.

Some derivatives were prepared by reacting Compound 1 with carboxylic acid derivatives, such as acyl halides, instead of carboxylic acids, or by further modifying amide derivatives bearing reactive functional groups, such as halogen or amino groups, in subsequent reactions.

In some reactions with carboxylic acids, solvents and/or bases different from those used in the general procedure were employed in view of solubility and/or reactivity. In certain embodiments, the reaction was carried out in the absence of a base catalyst.

For derivatives that are not prepared by the above general procedure, the preparation procedures are described in detail in the respective Examples.

The preparation of specific decursin amide derivatives is described in the following Examples.

### Example 1: Preparation of N-(8,8-dimethyl-2-oxo-7,8-dihydro-2H,6H-pyrano[3,2-g]chromen-7-yl)-3-methylbut-2-enamide (Compound 2)

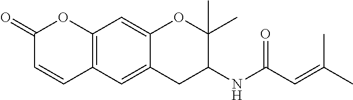 [SMILES: CC(C)=CC(=O)NC1Cc2cc3ccc(=O)oc3cc2OC1(C)C]

Compound 1 (120 mg, 0.49 mmol) and 3,3-dimethylacrylic acid (59 mg, 0.59 mmol) were reacted according to the method described in Preparation Example 2. The concentrated dichloromethane extract was purified by silica gel column chromatography using a mixture of dichloromethane and methanol (v/v, 50:1), affording Compound 2 as a white solid (104 mg, 65%).

Mp: 124° C.

¹H NMR (400 MHz, CDCl₃): δ 7.57 (d, J=9.5 Hz, 1H), 7.17 (s, 1H), 6.77 (s, 1H), 6.22 (d, J=9.5 Hz, 1H), 5.58 (d, J=9.1 Hz, 1H), 5.56-5.50 (m, 1H), 4.41-4.29 (m, 1H), 3.18 (ddd, J=16.9, 5.1, 0.8 Hz, 1H), 2.79 (dd, J=17.0, 3.0 Hz, 1H), 2.14 (d, J=1.0 Hz, 3H), 1.81 (d, J=1.0 Hz, 3H), 1.41 (s, 3H), 1.35 (s, 3H).

¹³C NMR (101 MHz, CDCl₃): δ 166.58, 161.14, 156.45, 154.12, 152.24, 143.07, 129.32, 118.05, 116.13, 113.54, 113.20, 105.03, 77.76, 47.40, 28.89, 27.15, 24.93, 24.73, 19.83.

MS (ESI): m/z [M+H]⁺ 328.1, found.

### Example 2: Preparation of (E)-N-(8,8-dimethyl-2-oxo-7,8-dihydro-2H,6H-pyrano[3,2-g]chromen-7-yl)cinnamamide (Compound 3)

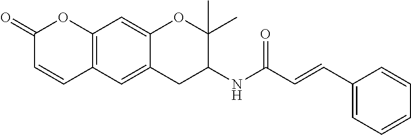 [SMILES: CC1(C)Oc2cc3oc(=O)ccc3cc2CC1NC(=O)/C=C/c1ccccc1]

Compound 1 (104 mg, 0.42 mmol) and trans-cinnamic acid (76 mg, 0.51 mmol) were reacted according to the method described in Preparation Example 2. The concentrated dichloromethane extract was purified by silica gel column chromatography using a mixture of dichloromethane and methanol (v/v, 50:1), affording Compound 3 as a white solid (127 mg, 81%).

Mp: 216-218° C.

¹H NMR (400 MHz, CDCl₃): δ 7.64 (d, J=15.6 Hz, 1H), 7.56 (d, J=9.5 Hz, 1H), 7.46 (dd, J=6.6, 2.8 Hz, 2H), 7.36-7.29 (m, 3H), 7.18 (s, 1H), 6.75 (s, 1H), 6.43 (d, J=15.6 Hz, 1H), 6.18 (d, J=9.5 Hz, 1H), 6.12 (d, J=9.2 Hz, 1H), 4.52-4.44 (m, 1H), 3.24 (dd, J=17.3, 4.8 Hz, 1H), 2.86 (dd, J=17.1, 2.6 Hz, 1H), 1.47 (s, 3H), 1.38 (s, 3H).

¹³C NMR (101 MHz, CDCl₃): δ 165.77, 161.18, 156.50, 154.09, 143.12, 141.76, 134.67, 129.81, 129.38, 128.80, 127.84, 120.21, 116.03, 113.48, 113.23, 105.07, 77.78, 48.01, 28.84, 24.93, 24.89.

MS (ESI): m/z [M+H]⁺ 376.1, found.

### Example 3: Preparation of (E)-N-(8,8-dimethyl-2-oxo-7,8-dihydro-2H,6H-pyrano[3,2-g]chromen-7-yl)-3-(4-fluorophenyl)acrylamide (Compound 4)

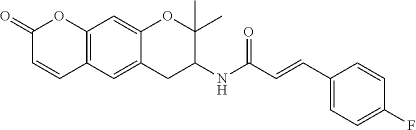 [SMILES: CC1(C)Oc2cc3oc(=O)ccc3cc2CC1NC(=O)/C=C/c1ccc(F)cc1]

Compound 1 (162 mg, 0.66 mmol) and 4-fluorocinnamic acid (132 mg, 0.79 mmol) were reacted according to the method described in Preparation Example 2. The concentrated dichloromethane extract was purified by silica gel column chromatography using a mixture of dichloromethane and methanol (v/v, 50:1), affording Compound 4 as a white solid (193 mg, 82%).

Mp: 187° C.

¹H NMR (400 MHz, CDCl₃): δ 7.60 (d, J=15.6 Hz, 1H), 7.56 (d, J=9.4 Hz, 1H), 7.44 (dd, J=8.7, 5.4 Hz, 2H), 7.19 (s, 1H), 7.02 (dd, J=12.0, 5.3 Hz, 2H), 6.74 (s, 1H), 6.36 (d, J=15.6 Hz, 1H), 6.16 (dd, J=16.6, 9.3 Hz, 2H), 4.48 (ddd, J=9.1, 5.1, 2.8 Hz, 1H), 3.24 (ddd, J=17.0, 5.1, 0.8 Hz, 1H), 2.86 (dd, J=17.1, 2.6 Hz, 1H), 1.47 (s, 3H), 1.38 (s, 3H).

¹³C NMR (101 MHz, DMSO): δ 165.35, 160.74, 156.68, 154.04, 144.60, 138.41, 132.14, 130.05, 130.03, 122.44, 117.71, 116.48, 116.26, 113.14, 113.04, 104.16, 78.58, 48.35, 28.49, 25.94, 22.97.

MS (ESI): m/z [M+H]⁺ 394.1, found.

### Example 4: Preparation of (E)-N-(8,8-dimethyl-2-oxo-7,8-dihydro-2H,6H-pyrano[3,2-g]chromen-7-yl)-3-(4-(trifluoromethyl)phenyl)acrylamide (Compound 5)

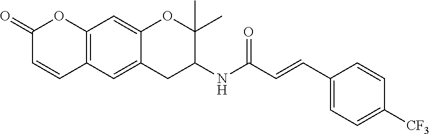 [SMILES: CC1(C)Oc2cc3oc(=O)ccc3cc2CC1NC(=O)/C=C/c1ccc(C(F)(F)F)cc1]

Compound 1 (115 mg, 0.47 mmol) and trans-4-(trifluoromethyl)cinnamic acid (121 mg, 0.56 mmol) were reacted according to the method described in Preparation Example 2. The concentrated dichloromethane extract was purified by silica gel column chromatography using a mixture of dichloromethane and methanol (v/v, 30:1), affording Compound 5 as a white solid (160 mg, 77%).

Mp: 189° C.

¹H NMR (400 MHz, DMSO): δ 8.24 (d, J=8.9 Hz, 1H), 7.94 (d, J=9.4 Hz, 1H), 7.77 (s, 4H), 7.53 (d, J=15.8 Hz, 1H), 7.50 (s, 1H), 6.93 (d, J=15.8 Hz, 1H), 6.83 (s, 1H), 6.28 (d, J=9.5 Hz, 1H), 4.35-4.21 (m, 1H), 3.13 (dd, J=17.2, 5.4 Hz, 1H), 2.78 (dd, J=17.1, 6.5 Hz, 1H), 1.33 (d, J=13.5 Hz, 6H).

¹³C NMR (101 MHz, DMSO): δ 164.96, 160.74, 156.65, 154.04, 144.60, 139.50, 137.91, 130.05, 128.57, 126.29, 126.25, 125.33, 117.61, 113.16, 113.06, 104.18, 78.51, 48.43, 31.41, 28.46, 25.89, 23.06, 22.52, 14.41.

MS (ESI): m/z [M+H]⁺ 444.1, found.

### Example 5: Preparation of (E)-N-(8,8-dimethyl-2-oxo-7,8-dihydro-2H,6H-pyrano[3,2-g]chromen-7-yl)-3-(4-nitrophenyl)acrylamide (Compound 6)

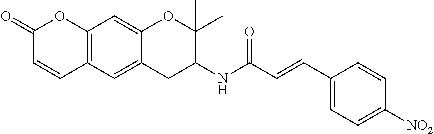 [SMILES: CC1(C)Oc2cc3oc(=O)ccc3cc2CC1NC(=O)/C=C/c1ccc([N+](=O)[O-])cc1]

Compound 1 (115 mg, 0.47 mmol) and 4-nitrocinnamic acid (108 mg, 0.56 mmol) were reacted according to the method described in Preparation Example 2. The concentrated dichloromethane extract was purified by silica gel column chromatography using a mixture of dichloromethane and methanol (v/v, 30:1), affording Compound 6 as a white solid (162 mg, 82%).

Mp: 239° C.

¹H NMR (400 MHz, DMSO): δ 8.27 (dd, J=11.4, 8.9 Hz, 3H), 7.94 (d, J=9.3 Hz, 1H), 7.80 (d, J=8.8 Hz, 2H), 7.57 (d, J=15.8 Hz, 1H), 7.50 (s, 1H), 6.99 (d, J=15.8 Hz, 1H), 6.83 (s, 1H), 6.28 (d, J=9.5 Hz, 1H), 4.37-4.19 (m, 1H), 3.13 (dd, J=17.2, 5.4 Hz, 1H), 2.78 (dd, J=17.2, 6.4 Hz, 1H), 1.33 (d, J=12.7 Hz, 6H).

¹³C NMR (101 MHz, DMSO): δ 164.75, 160.74, 156.64, 154.04, 147.98, 144.59, 142.03, 137.25, 130.06, 128.99, 126.82, 124.59, 117.56, 113.18, 113.07, 104.18, 78.48, 48.47, 28.44, 25.86, 23.11.

MS (ESI): m/z [M+H]⁺ 421.1, found.

### Example 6: Preparation of (E)-N-(8,8-dimethyl-2-oxo-7,8-dihydro-2H,6H-pyrano[3,2-g]chromen-7-yl)-3-(4-methoxyphenyl)acrylamide (Compound 7)

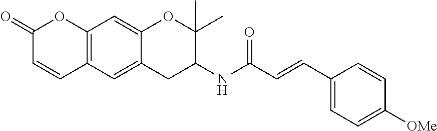 [SMILES: COc1ccc(/C=C/C(=O)NC2Cc3cc4ccc(=O)oc4cc3OC2(C)C)cc1]

Compound 1 (118 mg, 0.48 mmol) and 4-methoxycinnamic acid (103 mg, 0.58 mmol) were reacted according to the method described in Preparation Example 2. The concentrated dichloromethane extract was purified by silica gel column chromatography using a mixture of dichloromethane and methanol (v/v, 50:1), affording Compound 7 as a white solid (122 mg, 63%).

Mp: 201° C.

¹H NMR (400 MHz, CDCl₃): δ 7.50 (t, J=12.1 Hz, 2H), 7.33 (d, J=8.7 Hz, 2H), 7.11 (s, 1H), 6.78 (d, J=8.8 Hz, 2H), 6.70 (s, 1H), 6.20 (d, J=15.5 Hz, 1H), 6.12 (d, J=9.5 Hz, 1H), 5.89 (d, J=9.2 Hz, 1H), 4.40 (ddd, J=8.2, 5.0, 2.9 Hz, 1H), 3.73 (s, 3H), 3.15 (dd, J=17.3, 4.7 Hz, 1H), 2.78 (dd, J=17.1, 2.8 Hz, 1H), 1.39 (s, 3H), 1.30 (s, 3H).

¹³C NMR (101 MHz, CDCl₃): δ 166.09, 161.17, 161.02, 156.49, 154.11, 143.10, 141.41, 129.42, 129.38, 127.35, 117.74, 116.07, 114.25, 113.50, 113.23, 105.07, 77.81, 55.33, 47.95, 28.87, 24.90, 24.88.

MS (ESI): m/z [M+H]⁺ 406.1, found.

### Example 7: Preparation of (E)-N-(8,8-dimethyl-2-oxo-7,8-dihydro-2H,6H-pyrano[3,2-g]chromen-7-yl)-3-(p-tolyl)acrylamide (Compound 8)

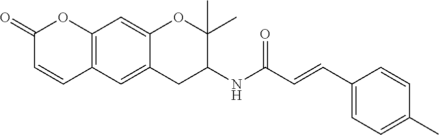 [SMILES: Cc1ccc(/C=C/C(=O)NC2Cc3cc4ccc(=O)oc4cc3OC2(C)C)cc1]

Compound 1 (105 mg, 0.43 mmol) and 4-methylcinnamic acid (84 mg, 0.52 mmol) were reacted according to the method described in Preparation Example 2. The concentrated dichloromethane extract was purified by silica gel column chromatography using a mixture of dichloromethane and methanol (v/v, 50:1), affording Compound 8 as a white solid (149 mg, 89%).

Mp: 188° C.

¹H NMR (400 MHz, CDCl₃): δ 7.58 (dd, J=18.8, 12.5 Hz, 2H), 7.36 (d, J=8.1 Hz, 2H), 7.18 (s, 1H), 7.14 (d, J=8.0 Hz, 2H), 6.77 (s, 1H), 6.36 (d, J=15.6 Hz, 1H), 6.19 (d, J=9.5 Hz, 1H), 6.02 (d, J=9.2 Hz, 1H), 4.47 (ddd, J=8.4, 5.0, 2.9 Hz, 1H), 3.23 (dd, J=17.4, 4.7 Hz, 1H), 2.86 (dd, J=17.1, 2.7 Hz, 1H), 2.34 (s, 3H), 1.46 (s, 3H), 1.38 (s, 3H).

¹³C NMR (101 MHz, CDCl₃): δ 165.95, 161.18, 156.48, 154.10, 143.12, 141.77, 140.17, 131.87, 129.54, 129.39, 127.83, 119.08, 116.04, 113.50, 113.23, 105.08, 77.79, 47.97, 28.85, 24.91, 24.90, 21.40.

MS (ESI): m/z [M+H]⁺ 390.1, found.

### Example 8: Preparation of (E)-3-(4-bromophenyl)-N-(8,8-dimethyl-2-oxo-7,8-dihydro-2H,6H-pyrano[3,2-g]chromen-7-yl)acrylamide (Compound 9)

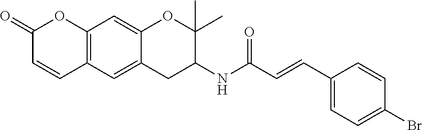 [SMILES: CC1(C)Oc2cc3oc(=O)ccc3cc2CC1NC(=O)/C=C/c1ccc(Br)cc1]

Compound 1 (105 mg, 0.43 mmol) and 4-bromocinnamic acid (118 mg, 0.52 mmol) were reacted according to the method described in Preparation Example 2. The concentrated dichloromethane extract was purified by silica gel column chromatography using a mixture of dichloromethane and methanol (v/v, 50:1), affording Compound 9 as a white solid (149 mg, 89%).

Mp: 228° C.

¹H NMR (400 MHz, CDCl₃): δ 7.57 (dd, J=12.5, 4.8 Hz, 2H), 7.46 (d, J=8.4 Hz, 2H), 7.32 (d, J=8.4 Hz, 2H), 7.18 (s, 1H), 6.72 (s, 1H), 6.45 (d, J=15.6 Hz, 1H), 6.25 (d, J=9.1 Hz, 1H), 6.17 (d, J=9.4 Hz, 1H), 4.52-4.43 (m, 1H), 3.24 (dd, J=17.1, 4.9 Hz, 1H), 2.91-2.81 (m, 1H), 1.47 (s, 3H), 1.37 (s, 3H).

¹³C NMR (101 MHz, CDCl₃): δ 165.50, 161.21, 156.52, 154.04, 143.16, 140.35, 133.65, 132.03, 129.40, 129.24, 123.92, 120.97, 116.01, 113.43, 113.20, 105.06, 77.76, 48.01, 28.82, 25.01, 24.88.

MS (ESI): m/z [M+H]⁺ 454.0, found.

### Example 9: Preparation of (E)-3-(4-chlorophenyl)-N-(8,8-dimethyl-2-oxo-7,8-dihydro-2H,6H-pyrano[3,2-g]chromen-7-yl)acrylamide (Compound 10)

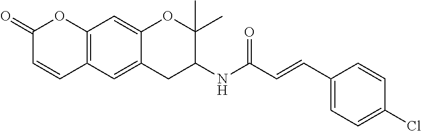 [SMILES: CC1(C)Oc2cc3oc(=O)ccc3cc2CC1NC(=O)/C=C/c1ccc(Cl)cc1]

Compound 1 (107 mg, 0.44 mmol) and 4-chlorocinnamic acid (97 mg, 0.53 mmol) were reacted according to the method described in Preparation Example 2. The concentrated dichloromethane extract was purified by silica gel column chromatography using a mixture of dichloromethane and methanol (v/v, 30:1), affording Compound 10 as a white solid (142 mg, 79%).

Mp: 207° C.

¹H NMR (400 MHz, CDCl₃): δ 7.57 (t, J=12.6 Hz, 2H), 7.39 (d, J=8.5 Hz, 2H), 7.29 (d, J=8.5 Hz, 2H), 7.18 (s, 1H), 6.72 (s, 1H), 6.43 (d, J=15.6 Hz, 1H), 6.26 (d, J=9.2 Hz, 1H), 6.17 (d, J=9.5 Hz, 1H), 4.50-4.45 (m, 1H), 3.24 (dd, J=17.1, 4.9 Hz, 1H), 2.86 (dd, J=17.1, 2.2 Hz, 1H), 1.47 (s, 3H), 1.37 (s, 3H).

¹³C NMR (101 MHz, CDCl₃): δ 164.42, 161.37, 156.55, 154.00, 145.12, 144.32, 143.32, 138.99, 135.35, 129.39, 125.57, 115.93, 113.37, 113.20, 105.01, 77.70, 48.30, 31.58, 28.74, 24.92, 24.88, 22.64, 14.10.

MS (ESI): m/z [M+H]⁺ 410.1, found.

### Example 10: Preparation of (E)-N-(8,8-dimethyl-2-oxo-7,8-dihydro-2H,6H-pyrano[3,2-g]chromen-7-yl)-3-(4-(dimethylamino)phenyl)acrylamide (Compound 11)

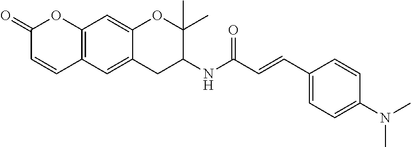 [SMILES: CN(C)c1ccc(/C=C/C(=O)NC2Cc3cc4ccc(=O)oc4cc3OC2(C)C)cc1]

Compound 1 (115 mg, 0.47 mmol) and 4-dimethylaminocinnamic acid (108 mg, 0.56 mmol) were reacted according to the method described in Preparation Example 2. The concentrated dichloromethane extract was purified by silica gel column chromatography using a mixture of dichloromethane and methanol (v/v, 30:1), affording Compound 11 as an ivory solid (115 mg, 58%).

Mp: 244.2° C.

¹H NMR (400 MHz, DMSO): δ 7.93 (dd, J=9.2, 5.6 Hz, 2H), 7.48 (s, 1H), 7.35 (dd, J=12.1, 9.1 Hz, 3H), 6.81 (s, 1H), 6.71 (d, J=8.9 Hz, 2H), 6.51 (d, J=15.7 Hz, 1H), 6.27 (d, J=9.5 Hz, 1H), 4.27 (dd, J=14.5, 6.8 Hz, 1H), 3.08 (dd, J=17.0, 5.4 Hz, 1H), 2.95 (s, 6H), 2.75 (dd, J=17.0, 7.0 Hz, 1H), 1.31 (d, J=17.4 Hz, 6H).

¹³C NMR (101 MHz, DMSO): δ 166.15, 160.76, 156.73, 154.02, 151.57, 144.62, 140.13, 129.99, 129.32, 122.81, 117.90, 116.86, 113.09, 112.99, 112.44, 104.13, 78.72, 48.21, 28.57, 26.05, 22.78.

MS (ESI): m/z [M+H]⁺ 419.2, found.

### Example 11: Preparation of (E)-3-(benzo[d][1,3]dioxol-5-yl)-N-(8,8-dimethyl-2-oxo-7,8-dihydro-2H,6H-pyrano[3,2-g]chromen-7-yl)acrylamide (Compound 12)

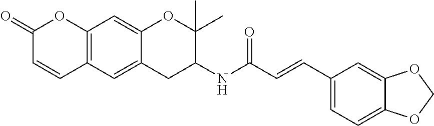 [SMILES: CC1(C)Oc2cc3oc(=O)ccc3cc2CC1NC(=O)/C=C/c1ccc2c(c1)OCO2]

Compound 1 (100 mg, 0.41 mmol) and 3,4-(methylenedioxy)cinnamic acid (95 mg, 0.49 mmol) were reacted according to the method described in Preparation Example 2. The concentrated dichloromethane extract was purified by silica gel column chromatography using a mixture of dichloromethane and methanol (v/v, 50:1), affording Compound 12 as a white solid (122 mg, 71%).

Mp: 265° C.

¹H NMR (400 MHz, CDCl₃): δ 7.55 (dd, J=12.5, 7.9 Hz, 2H), 7.18 (s, 1H), 6.95 (dd, J=4.1, 2.5 Hz, 2H), 6.77 (d, J=7.3 Hz, 2H), 6.22 (dd, J=18.1, 12.5 Hz, 2H), 6.02 (d, J=9.2 Hz, 1H), 5.97 (s, 2H), 4.51-4.42 (m, 1H), 3.23 (dd, J=17.2, 4.8 Hz, 1H), 2.85 (dd, J=17.1, 2.7 Hz, 1H), 1.46 (s, 3H), 1.37 (s, 3H).

¹³C NMR (101 MHz, CDCl₃): δ 165.91, 161.18, 156.48, 154.09, 149.19, 148.24, 143.12, 141.48, 129.38, 129.06, 123.98, 118.19, 116.04, 113.49, 113.22, 108.51, 106.36, 105.07, 101.45, 77.79, 47.96, 28.86, 24.91, 24.90.

MS (ESI): m/z [M+H]⁺ 420.1, found.

### Example 12: Preparation of (E)-N-(8,8-dimethyl-2-oxo-7,8-dihydro-2H,6H-pyrano[3,2-g]chromen-7-yl)-3-(4-hydroxy-3-methoxyphenyl)acrylamide (Compound 13)

### Preparation of (E)-4-(3-((8,8-dimethyl-2-oxo-7,8-dihydro-2H,6H-pyrano[3,2-g]chromen-7-yl)amino)-3-oxoprop-1-en-1-yl)-2-methoxyphenyl acetate (Compound 13a)

Compound 1 (200 mg, 0.82 mmol) and 4-acetoxy-3-methoxycinnamic acid (231 mg, 0.98 mmol) were reacted according to the method described in Preparation Example 2. The concentrated dichloromethane extract was purified by silica gel column chromatography using a mixture of dichloromethane and methanol (v/v, 50:1), affording Compound 13a as an ivory solid (240 mg, 63%).

¹H NMR (400 MHz, CDCl₃): δ 7.58 (dd, J=12.5, 6.4 Hz, 2H), 7.20 (s, 1H), 7.10-6.97 (m, 3H), 6.79 (s, 1H), 6.32 (d, J=15.5 Hz, 1H), 6.22 (d, J=9.5 Hz, 1H), 5.91 (d, J=9.2 Hz, 1H), 4.52-4.43 (m, 1H), 3.81 (s, 3H), 3.24 (dd, J=17.5, 4.8 Hz, 1H), 2.86 (dd, J=17.2, 2.7 Hz, 1H), 2.30 (s, 3H), 1.46 (s, 3H), 1.38 (s, 3H).

### Preparation of (E)-N-(8,8-dimethyl-2-oxo-7,8-dihydro-2H,6H-pyrano[3,2-g]chromen-7-yl)-3-(4-hydroxy-3-methoxyphenyl)acrylamide (Compound 13)

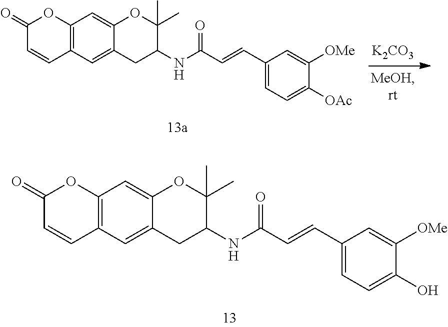

Compound 13a (230 mg, 0.50 mmol) was dissolved in methanol, and potassium carbonate (76 mg, 0.55 mmol) was added, followed by stirring at room temperature for 1 hour. Upon completion of the reaction, 1 N hydrochloric acid was added to quench the reaction, and the mixture was extracted with ethyl acetate. The ethyl acetate extract was dried over anhydrous sodium sulfate and concentrated, and the residue was purified by silica gel column chromatography using a mixture of dichloromethane and methanol (v/v, 30:1), affording Compound 13 as a white solid (144 mg, 68%).

Mp: 213° C.

¹H NMR (400 MHz, DMSO): δ 9.42 (s, 1H), 7.98 (d, J=8.9 Hz, 1H), 7.93 (d, J=9.5 Hz, 1H), 7.48 (s, 1H), 7.35 (d, J=15.7 Hz, 1H), 7.10 (d, J=1.6 Hz, 1H), 6.97 (dd, J=8.2, 1.7 Hz, 1H), 6.82 (s, 1H), 6.78 (d, J=8.1 Hz, 1H), 6.59 (d, J=15.7 Hz, 1H), 6.27 (d, J=9.5 Hz, 1H), 4.27 (dt, J=8.6, 6.4 Hz, 1H), 3.79 (s, 3H), 3.09 (dd, J=17.1, 5.4 Hz, 1H), 2.75 (dd, J=17.0, 6.8 Hz, 1H), 1.34 (s, 3H), 1.30 (s, 3H).

¹³C NMR (101 MHz, DMSO): δ 165.84, 160.76, 156.71, 154.03, 148.82, 148.30, 144.61, 140.02, 130.01, 126.86, 122.36, 119.15, 117.80, 116.07, 113.11, 113.02, 110.80, 104.15, 78.65, 55.91, 48.24, 28.54, 25.98, 22.92.

MS (ESI): m/z [M+H]⁺ 422.1, found.

### Example 13: Preparation of (E)-N-(8,8-dimethyl-2-oxo-7,8-dihydro-2H,6H-pyrano[3,2-g]chromen-7-yl)-3-(pyridin-3-yl)acrylamide (Compound 14)

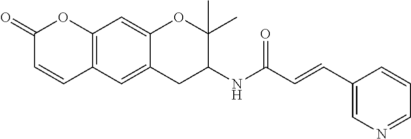 [SMILES: CC1(C)Oc2cc3oc(=O)ccc3cc2CC1NC(=O)/C=C/c1cccnc1]

Compound 1 (120 mg, 0.49 mmol) and trans-3-(3-pyridyl)acrylic acid (88 mg, 0.59 mmol) were reacted according to the method described in Preparation Example 2. The dichloromethane extract was concentrated, and the residue was purified by silica gel column chromatography using a mixture of dichloromethane and methanol (v/v, 30:1), affording Compound 14 as a white solid (173 mg, 94%).

Mp: 242° C.

¹H NMR (400 MHz, CDCl₃): δ 8.69 (d, J=1.7 Hz, 1H), 8.53 (dd, J=4.8, 1.4 Hz, 1H), 7.75 (dt, J=8.0, 1.8 Hz, 1H), 7.63 (d, J=15.7 Hz, 1H), 7.57 (d, J=9.5 Hz, 1H), 7.29-7.24 (m, 1H), 7.20 (s, 1H), 6.71 (s, 1H), 6.53 (d, J=15.7 Hz, 1H), 6.31 (d, J=9.2 Hz, 1H), 6.19 (d, J=9.5 Hz, 1H), 4.49 (ddd, J=9.0, 5.1, 2.7 Hz, 1H), 3.30-3.21 (m, 1H), 2.87 (dd, J=17.2, 2.5 Hz, 1H), 1.47 (s, 3H), 1.38 (s, 3H).

¹³C NMR (101 MHz, CDCl₃): δ 164.41, 161.37, 156.53, 154.01, 145.11, 144.32, 143.31, 139.02, 135.37, 129.39, 125.55, 115.9, 113.39, 113.21, 105.02, 48.31, 31.58, 28.74, 24.91, 24.88, 22.64, 14.10.

MS (ESI): m/z [M+H]⁺ 377.1, found.

### Example 14: Preparation of (E)-N-(8,8-dimethyl-2-oxo-7,8-dihydro-2H,6H-pyrano[3,2-g]chromen-7-yl)-3-(furan-2-yl)acrylamide (Compound 15)

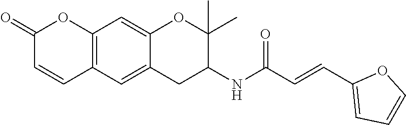 [SMILES: CC1(C)Oc2cc3oc(=O)ccc3cc2CC1NC(=O)/C=C/c1ccco1]

Compound 1 (150 mg, 0.61 mmol) and 3-(2-furyl)acrylic acid (101 mg, 0.73 mmol) were reacted according to the method described in Preparation Example 2. The dichloromethane extract was concentrated, and the residue was purified by silica gel column chromatography using a mixture of dichloromethane and methanol (v/v, 30:1), affording Compound 15 as a white solid (132 mg, 59%).

Mp: 228° C.

¹H NMR (400 MHz, CDCl₃): δ 7.56 (d, J=9.5 Hz, 1H), 7.39 (dd, J=8.3, 6.8 Hz, 2H), 7.18 (s, 1H), 6.77 (s, 1H), 6.53 (d, J=3.4 Hz, 1H), 6.42 (dd, J=3.4, 1.8 Hz, 1H), 6.29 (d, J=15.3 Hz, 1H), 6.20 (d, J=9.5 Hz, 1H), 5.97 (d, J=9.2 Hz, 1H), 4.49-4.42 (m, 1H), 3.22 (ddd, J=17.0, 5.0, 0.6 Hz, 1H), 2.84 (dd, J=17.1, 2.9 Hz, 1H), 1.44 (s, 3H), 1.37 (s, 3H).

¹³C NMR (101 MHz, CDCl₃): δ 165.66, 161.14, 156.42, 154.12, 151.16, 144.13, 143.06, 129.34, 128.54, 117.87, 115.98, 114.02, 113.55, 113.25, 112.15, 105.08, 77.74, 48.05, 28.83, 24.90, 24.80.

MS (ESI): m/z [M+H]⁺ 366.1, found.

### Example 15: Preparation of (E)-N-(8,8-dimethyl-2-oxo-7,8-dihydro-2H,6H-pyrano[3,2-g]chromen-7-yl)-3-(thiophen-2-yl)acrylamide (Compound 16)

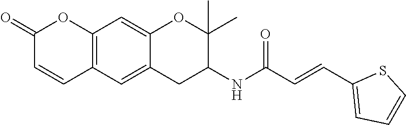 [SMILES: CC1(C)Oc2cc3oc(=O)ccc3cc2CC1NC(=O)/C=C/c1cccs1]

Compound 1 (150 mg, 0.61 mmol) and trans-3-(3-thienyl)acrylic acid (113 mg, 0.73 mmol) were reacted according to the method described in Preparation Example 2. The dichloromethane extract was concentrated, and the residue was purified by silica gel column chromatography using a mixture of dichloromethane and methanol (v/v, 30:1), affording Compound 16 as a white solid (147 mg, 63%).

Mp: 249° C.

¹H NMR (400 MHz, CDCl₃): δ 7.62 (d, J=15.5 Hz, 1H), 7.56 (d, J=9.5 Hz, 1H), 7.42 (d, J=2.0 Hz, 1H), 7.30-7.25 (m, 1H), 7.23-7.16 (m, 2H), 6.76 (s, 1H), 6.24 (d, J=15.5 Hz, 1H), 6.19 (d, J=9.5 Hz, 1H), 6.01 (d, J=9.2 Hz, 1H), 4.51-4.42 (m, 1H), 3.23 (dd, J=17.1, 4.8 Hz, 1H), 2.85 (dd, J=17.1, 2.6 Hz, 1H), 1.46 (s, 3H), 1.37 (s, 3H).

¹³C NMR (101 MHz, CDCl₃): δ 165.97, 161.15, 156.47, 154.10, 143.09, 137.67, 135.36, 129.38, 127.31, 126.79, 125.03, 119.86, 116.02, 113.51, 113.24, 105.07, 77.77, 47.98, 28.85, 24.92, 24.89.

MS (ESI): m/z [M+H]⁺ 382.1, found.

### Example 16: Preparation of N-(8,8-dimethyl-2-oxo-7,8-dihydro-2H,6H-pyrano[3,2-g]chromen-7-yl)-4-phenylbutanamide (Compound 17)

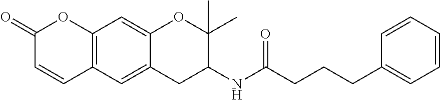 [SMILES: CC1(C)Oc2cc3oc(=O)ccc3cc2CC1NC(=O)CCCc1ccccc1]

Compound 1 (109 mg, 0.44 mmol) and 4-phenylbutyric acid (87 mg, 0.53 mmol) were reacted according to the method described in Preparation Example 2. The dichloromethane extract was concentrated, and the residue was purified by silica gel column chromatography using a mixture of dichloromethane and methanol (v/v, 30:1), affording Compound 17 as a white solid (124 mg, 72%).

Mp: 144-146° C.

¹H NMR (400 MHz, DMSO): δ 7.92 (d, J=9.5 Hz, 1H), 7.88 (d, J=8.9 Hz, 1H), 7.46 (s, 1H), 7.27 (t, J=7.5 Hz, 2H), 7.17 (t, J=6.9 Hz, 3H), 6.78 (s, 1H), 6.26 (d, J=9.5 Hz, 1H), 4.15 (td, J=8.0, 6.0 Hz, 1H), 2.99 (dd, J=16.9, 5.5 Hz, 1H), 2.70 (dd, J=16.9, 7.8 Hz, 1H), 2.55 (t, J=7.6 Hz, 2H), 2.22-2.08 (m, 2H), 1.86-1.73 (m, 2H), 1.31 (s, 3H), 1.24 (s, 3H).

¹³C NMR (101 MHz, DMSO): δ 172.47, 160.75, 156.67, 154.00, 144.60, 142.20, 129.85, 128.77, 128.74, 126.22, 118.05, 113.08, 112.94, 104.09, 78.69, 48.11, 35.14, 28.39, 27.65, 26.14, 22.30.

MS (ESI): m/z [M+H]⁺ 392.1, found.

### Example 17: Preparation of N-(8,8-dimethyl-2-oxo-7,8-dihydro-2H,6H-pyrano[3,2-g]chromen-7-yl)cyclopentanecarboxamide (Compound 18)

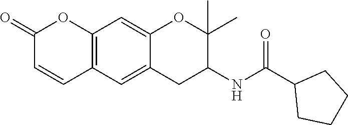 [SMILES: CC1(C)Oc2cc3oc(=O)ccc3cc2CC1NC(=O)C1CCCC1]

Compound 1 (123 mg, 0.50 mmol) and cyclopentanecarboxylic acid (65 μL, 0.60 mmol) were reacted according to the method described in Preparation Example 2. The dichloromethane extract was concentrated, and the residue was purified by silica gel column chromatography using a mixture of dichloromethane and methanol (v/v, 50:1), affording Compound 18 as a white solid (112 mg, 66%).

Mp: 215° C.

¹H NMR (400 MHz, DMSO): δ 7.93 (d, J=9.5 Hz, 1H), 7.80 (d, J=9.0 Hz, 1H), 7.46 (s, 1H), 6.78 (s, 1H), 6.26 (d, J=9.5 Hz, 1H), 4.14 (td, J=8.4, 5.7 Hz, 1H), 2.98 (dd, J=17.0, 5.6 Hz, 1H), 2.76-2.57 (m, 2H), 1.77 (s, 1H), 1.63 (s, 5H), 1.48 (s, 2H), 1.29 (s, 3H), 1.23 (s, 3H).

¹³C NMR (101 MHz, DMSO): δ 175.87, 160.75, 156.67, 154.00, 144.61, 129.80, 118.16, 113.08, 112.92, 104.08, 78.84, 48.06, 44.38, 31.00, 29.96, 28.35, 26.19, 26.16, 26.09, 22.05.

MS (ESI): m/z [M+H]⁺ 342.2, found.

### Example 18: Preparation of 2-cyclopentyl-N-(8,8-dimethyl-2-oxo-7,8-dihydro-2H,6H-pyrano[3,2-g]chromen-7-yl)acetamide (Compound 19)

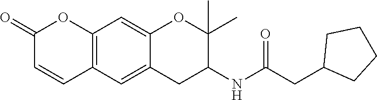 [SMILES: CC1(C)Oc2cc3oc(=O)ccc3cc2CC1NC(=O)CC1CCCC1]

Compound 1 (105 mg, 0.43 mmol) and cyclopentylacetic acid (65 μL, 0.52 mmol) were reacted according to the method described in Preparation Example 2. The dichloromethane extract was concentrated, and the residue was purified by silica gel column chromatography using a mixture of dichloromethane and methanol (v/v, 30:1), affording Compound 19 as a white solid (136 mg, 89%).

Mp: 150° C.

¹H NMR (400 MHz, DMSO): δ 7.92 (d, J=9.5 Hz, 1H), 7.82 (d, J=8.9 Hz, 1H), 7.46 (s, 1H), 6.78 (s, 1H), 6.26 (d, J=9.5 Hz, 1H), 4.13 (td, J=8.2, 5.8 Hz, 1H), 2.98 (dd, J=16.9, 5.6 Hz, 1H), 2.68 (dd, J=16.9, 7.9 Hz, 1H), 2.18-2.08 (m, 3H), 1.67 (s, 2H), 1.60-1.44 (m, 4H), 1.30 (s, 3H), 1.23 (s, 3H), 1.12 (s, 2H).

¹³C NMR (101 MHz, DMSO): δ 172.32, 160.75, 156.68, 154.00, 144.60, 129.82, 118.08, 113.08, 112.93, 104.08, 78.71, 48.05, 41.87, 37.29, 32.30, 28.45, 26.20, 25.01, 24.99, 22.21.

MS (ESI): m/z [M+H]⁺ 356.2, found.

### Example 19: Preparation of N-(8,8-dimethyl-2-oxo-7,8-dihydro-2H,6H-pyrano[3,2-g]chromen-7-yl)acetamide (Compound 20)

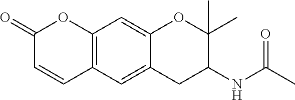 [SMILES: CC(=O)NC1Cc2cc3ccc(=O)oc3cc2OC1(C)C]

Compound 1 (130 mg, 0.53 mmol) and acetic acid (37 μL, 0.64 mmol) were reacted according to the method described in Preparation Example 2. The dichloromethane extract was concentrated, and the residue was purified by silica gel column chromatography using a mixture of dichloromethane and methanol (v/v, 50:1), affording Compound 20 as a white solid (107 mg, 70%).

Mp: 228° C.

¹H NMR (400 MHz, CDCl₃): δ 7.57 (d, J=9.5 Hz, 1H), 7.18 (s, 1H), 6.73 (s, 1H), 6.20 (d, J=9.4 Hz, 1H), 5.88 (d, J=8.3 Hz, 1H), 4.40-4.26 (m, 1H), 3.18 (dd, J=17.0, 4.8 Hz, 1H), 2.78 (dd, J=16.9, 1.7 Hz, 1H), 1.99 (s, 3H), 1.42 (s, 3H), 1.34 (s, 3H).

¹³C NMR (101 MHz, CDCl₃): δ 169.96, 161.11, 156.41, 154.09, 143.07, 129.33, 115.96, 113.55, 113.23, 105.06, 77.61, 47.88, 28.77, 24.83, 23.28.

MS (ESI): m/z [M+H]⁺: 288.1, found.

### Example 20: Preparation of N-(8,8-dimethyl-2-oxo-7,8-dihydro-2H,6H-pyrano[3,2-g]chromen-7-yl)heptanamide (Compound 21)

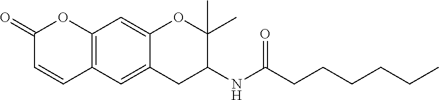 [SMILES: CCCCCCC(=O)NC1Cc2cc3ccc(=O)oc3cc2OC1(C)C]

Compound 1 (130 mg, 0.53 mmol) and heptanoic acid (79 μL, 0.64 mmol) were reacted according to the method described in Preparation Example 2. The dichloromethane extract was concentrated, and the residue was purified by silica gel column chromatography using a mixture of dichloromethane and methanol (v/v, 50:1), affording Compound 21 as a white solid (128 mg, 68%).

Mp: 86-88° C.

¹H NMR (400 MHz, CDCl₃): δ 7.58 (d, J=9.5 Hz, 1H), 7.18 (s, 1H), 6.75 (s, 1H), 6.21 (d, J=9.5 Hz, 1H), 5.74 (d, J=9.1 Hz, 1H), 4.36-4.29 (m, 1H), 3.17 (dd, J=17.1, 5.1 Hz, 1H), 2.77 (dd, J=17.1, 2.9 Hz, 1H), 2.21-2.11 (m, 2H), 1.58 (dd, J=14.5, 7.1 Hz, 2H), 1.41 (s, 3H), 1.34 (s, 3H), 1.25 (d, J=3.9 Hz, 6H), 0.84 (t, J=6.7 Hz, 3H).

¹³C NMR (101 MHz, CDCl₃): δ 173.05, 161.09, 156.40, 154.10, 143.05, 129.29, 116.03, 113.56, 113.23, 105.05, 77.63, 47.68, 36.76, 31.45, 28.87, 28.84, 25.71, 24.86, 24.74, 22.47, 13.97.

MS (ESI): m/z [M+H]⁺: 358.2, found.

### Example 21: Preparation of (E)-3-(4-aminophenyl)-N-(8,8-dimethyl-2-oxo-7,8-dihydro-2H,6H-pyrano[3,2-g]chromen-7-yl)acrylamide (Compound 22)

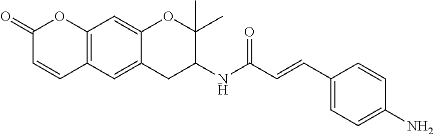 [SMILES: CC1(C)Oc2cc3oc(=O)ccc3cc2CC1NC(=O)/C=C/c1ccc(N)cc1]

Compound 1 (130 mg, 0.53 mmol) and trans-4-aminocinnamic acid (104 mg, 0.64 mmol) were reacted according to the method described in Preparation Example 2. The dichloromethane extract was concentrated, and the residue was purified by silica gel column chromatography using a mixture of dichloromethane and methanol (v/v, 50:1), affording Compound 22 as a yellow solid (98 mg, 47%).

Mp: 253° C.

¹H NMR (400 MHz, DMSO): δ 7.93 (d, J=9.5 Hz, 1H), 7.88 (d, J=8.9 Hz, 1H), 7.48 (s, 1H), 7.28 (d, J=15.6 Hz, 1H), 7.21 (d, J=8.5 Hz, 2H), 6.81 (s, 1H), 6.55 (d, J=8.5 Hz, 2H), 6.43 (d, J=15.6 Hz, 1H), 6.26 (d, J=9.5 Hz, 1H), 4.25 (dd, J=14.5, 7.0 Hz, 1H), 3.07 (dd, J=17.0, 5.4 Hz, 1H), 2.74 (dd, J=17.0, 7.2 Hz, 1H), 1.31 (d, J=18.9 Hz, 6H).

¹³C NMR (101 MHz, DMSO): δ 166.27, 160.78, 156.73, 154.02, 150.98, 144.63, 140.51, 129.97, 129.53, 122.70, 117.93, 116.10, 114.16, 113.08, 112.97, 104.12, 78.75, 48.19, 28.57, 26.07, 22.71.

MS (ESI): m/z [M+H]⁺ 391.2, found.

### Example 22: Preparation of methyl (5-((8,8-dimethyl-2-oxo-7,8-dihydro-2H,6H-pyrano[3,2-g]chromen-7-yl)carbamoyl)-1H-benzo[d]imidazol-2-yl)carbamate (Compound 23)

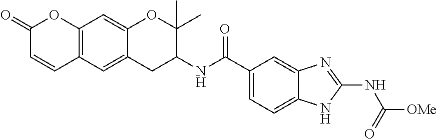 [SMILES: COC(=O)Nc1nc2cc(C(=O)NC3Cc4cc5ccc(=O)oc5cc4OC3(C)C)ccc2[nH]1]

Compound 1 (200 mg, 0.82 mmol) and 2-methoxycarbonylamino-1H-benzoimidazole-6-carboxylic acid (230 mg, 0.98 mmol) were reacted according to the method described in Preparation Example 2. The dichloromethane extract was concentrated, and the residue was purified by silica gel column chromatography using a mixture of dichloromethane and methanol (v/v, 50:1), affording Compound 23 as a white solid (110 mg, 29%).

Mp: 276-278° C.

¹H NMR (400 MHz, DMSO): δ 11.76 (br, 2H), 8.22 (d, J=8.9 Hz, 1H), 7.94 (d, J=8.7 Hz, 2H), 7.68-7.62 (m, 1H), 7.50 (s, 1H), 7.44 (d, J=8.3 Hz, 1H), 6.81 (s, 1H), 6.27 (d, J=9.5 Hz, 1H), 4.45 (q, J=8.1 Hz, 1H), 3.77 (s, 3H), 3.01 (d, J=7.8 Hz, 2H), 1.39 (s, 3H), 1.31 (s, 3H).

¹³C NMR (101 MHz, DMSO): δ 167.90, 160.80, 156.67, 154.01, 144.65, 129.66, 127.92, 121.41, 118.72, 113.09, 112.92, 104.09, 79.35, 53.03, 49.14, 28.04, 26.59, 21.41.

MS (ESI): m/z [M+H]⁺ 463.2, found.

### Example 23: Preparation of N-(8,8-dimethyl-2-oxo-7,8-dihydro-2H,6H-pyrano[3,2-g]chromen-7-yl)benzamide (Compound 24)

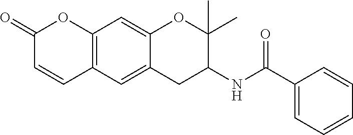 [SMILES: CC1(C)Oc2cc3oc(=O)ccc3cc2CC1NC(=O)c1ccccc1]

Compound 1 (70 mg, 0.29 mmol) and benzoic acid (43 mg, 0.35 mmol) were reacted according to the method described in Preparation Example 2. The dichloromethane extract was concentrated, and the residue was purified by silica gel column chromatography using a mixture of dichloromethane and methanol (v/v, 100:1), affording Compound 24 as a white solid (53 mg, 52%).

Mp: 207° C.

¹H NMR (400 MHz, CDCl₃): δ 7.71-7.66 (m, 2H), 7.58 (d, J=9.5 Hz, 1H), 7.48 (ddt, J=12.0, 7.8, 3.9 Hz, 1H), 7.40 (t, J=7.5 Hz, 2H), 7.20 (s, 1H), 6.84 (s, 1H), 6.30-6.18 (m, 2H), 4.56-4.50 (m, 1H), 3.27 (dd, J=17.1, 5.0 Hz, 1H), 2.92 (dd, J=17.1, 2.9 Hz, 1H), 1.49 (s, 3H), 1.41 (s, 3H).

¹³C NMR (101 MHz, CDCl₃): δ 167.25, 161.06, 156.28, 154.20, 143.01, 134.05, 131.83, 129.38, 128.68, 126.90, 115.86, 113.76, 113.42, 105.20, 77.72, 48.44, 28.82, 24.89, 24.86.

MS (ESI): m/z [M+H]⁺ 350.1, found.

### Example 24: Preparation of 2-bromo-N-(8,8-dimethyl-2-oxo-7,8-dihydro-2H,6H-pyrano[3,2-g]chromen-7-yl)benzamide (Compound 25)

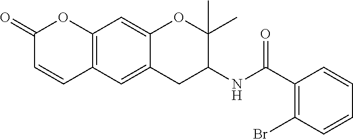 [SMILES: CC1(C)Oc2cc3oc(=O)ccc3cc2CC1NC(=O)c1ccccc1Br]

Compound 1 (70 mg, 0.29 mmol) and 2-bromobenzoic acid (70 mg, 0.35 mmol) were reacted according to the method described in Preparation Example 2. The dichloromethane extract was concentrated, and the residue was purified by silica gel column chromatography using a mixture of dichloromethane and methanol (v/v, 100:1), affording Compound 25 as a white solid (65 mg, 52%).

Mp: 170-172° C.

¹H NMR (400 MHz, CDCl₃): δ 7.58 (d, J=9.5 Hz, 1H), 7.52 (ddd, J=11.1, 7.8, 1.4 Hz, 2H), 7.33 (td, J=7.5, 1.1 Hz, 1H), 7.25 (td, J=7.7, 1.8 Hz, 1H), 7.21 (s, 1H), 6.79 (s, 1H), 6.23 (d, J=9.5 Hz, 1H), 6.19 (d, J=9.0 Hz, 1H), 4.56-4.49 (m, 1H), 3.27 (ddd, J=17.0, 5.0, 0.8 Hz, 1H), 2.99 (dd, J=17.1, 3.2 Hz, 1H), 1.52 (s, 3H), 1.42 (s, 3H).

¹³C NMR (101 MHz, CDCl₃): δ 167.31, 161.05, 156.26, 154.19, 143.01, 137.21, 133.44, 131.54, 129.69, 129.22, 127.66, 119.08, 115.87, 113.74, 113.35, 105.15, 77.52, 48.91, 28.69, 24.94, 24.83.

MS (ESI): m/z [M+H]⁺ 428.0, found.

### Example 25: Preparation of N-(8,8-dimethyl-2-oxo-7,8-dihydro-2H,6H-pyrano[3,2-g]chromen-7-yl)-2-iodobenzamide (Compound 26)

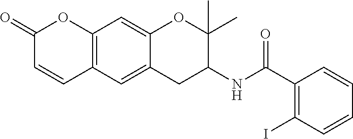 [SMILES: CC1(C)Oc2cc3oc(=O)ccc3cc2CC1NC(=O)c1ccccc1I]

Compound 1 (70 mg, 0.29 mmol) and 2-iodobenzoic acid (87 mg, 0.35 mmol) were reacted according to the method described in Preparation Example 2. The dichloromethane extract was concentrated, and the residue was purified by silica gel column chromatography using a mixture of dichloromethane and methanol (v/v, 100:1), affording Compound 26 as a white solid (65 mg, 47%).

Mp: 182° C.

¹H NMR (400 MHz, CDCl₃): δ 7.81 (d, J=7.9 Hz, 1H), 7.58 (d, J=9.5 Hz, 1H), 7.38-7.31 (m, 2H), 7.22 (s, 1H), 7.08 (ddd, J=8.1, 6.3, 2.9 Hz, 1H), 6.79 (s, 1H), 6.23 (d, J=9.5 Hz, 1H), 5.95 (d, J=9.0 Hz, 1H), 4.55-4.49 (m, 1H), 3.27 (dd, J=17.1, 5.0 Hz, 1H), 3.04 (dd, J=17.1, 3.1 Hz, 1H), 1.53 (s, 3H), 1.42 (s, 3H).

¹³C NMR (101 MHz, CDCl₃): δ 169.09, 161.04, 156.26, 154.19, 143.00, 141.69, 139.93, 131.37, 129.27, 128.30, 128.27, 115.85, 113.76, 113.40, 105.19, 92.14, 77.50, 48.79, 28.62, 24.96, 24.94.

MS (ESI): m/z [M+H]⁺ 476.0, found.

### Example 26: Preparation of N-(8,8-dimethyl-2-oxo-7,8-dihydro-2H,6H-pyrano[3,2-g]chromen-7-yl)-2-methoxybenzamide (Compound 27)

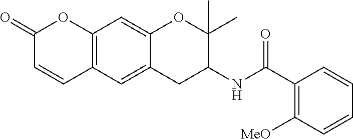 [SMILES: COc1ccccc1C(=O)NC1Cc2cc3ccc(=O)oc3cc2OC1(C)C]

Compound 1 (70 mg, 0.29 mmol) and 2-methoxybenzoic acid (53 mg, 0.35 mmol) were reacted according to the method described in Preparation Example 2. The dichloromethane extract was concentrated, and the residue was purified by silica gel column chromatography using a mixture of dichloromethane and methanol (v/v, 100:1), affording Compound 27 as a white solid (58 mg, 53%).

Mp: 132-133° C.

¹H NMR (400 MHz, CDCl₃): δ 8.22 (d, J=8.7 Hz, 1H), 8.17 (dd, J=7.8, 1.8 Hz, 1H), 7.59 (d, J=9.5 Hz, 1H), 7.42 (ddd, J=8.4, 7.5, 1.8 Hz, 1H), 7.20 (s, 1H), 7.10-7.03 (m, 1H), 6.92-6.83 (m, 2H), 6.24 (d, J=9.5 Hz, 1H), 4.51 (ddd, J=8.4, 4.7, 3.3 Hz, 1H), 3.71 (s, 3H), 3.23 (ddd, J=16.8, 4.7, 0.7 Hz, 1H), 2.93 (dd, J=16.9, 3.2 Hz, 1H), 1.48 (s, 3H), 1.41 (s, 3H).

¹³C NMR (101 MHz, CDCl₃): δ 164.97, 161.20, 157.44, 156.64, 154.18, 143.16, 133.02, 132.14, 129.51, 121.43, 121.12, 116.42, 113.52, 113.09, 111.45, 104.63, 77.78, 55.85, 48.44, 28.86, 24.93, 24.87.

MS (ESI): m/z [M+H]⁺ 380.1, found.

### Example 27: Preparation of 2-chloro-N-(8,8-dimethyl-2-oxo-7,8-dihydro-2H,6H-pyrano[3,2-g]chromen-7-yl)benzamide (Compound 28)

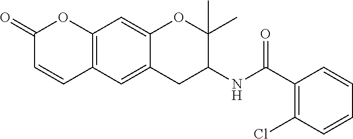 [SMILES: CC1(C)Oc2cc3oc(=O)ccc3cc2CC1NC(=O)c1ccccc1Cl]

Compound 1 (70 mg, 0.29 mmol) and 2-chlorobenzoic acid (55 mg, 0.35 mmol) were reacted according to the method described in Preparation Example 2. The dichloromethane extract was concentrated, and the residue was purified by silica gel column chromatography using a mixture of dichloromethane and methanol (v/v, 100:1), affording Compound 28 as a white solid (59 mg, 53%).

Mp: 185° C.

¹H NMR (400 MHz, CDCl₃): δ 7.66-7.54 (m, 2H), 7.38-7.28 (m, 3H), 7.21 (s, 1H), 6.38 (d, J=8.9 Hz, 1H), 6.23 (d, J=9.5 Hz, 1H), 4.53 (ddd, J=8.8, 5.0, 3.5 Hz, 1H), 3.26 (dd, J=17.0, 5.0 Hz, 1H), 2.96 (dd, J=17.1, 3.3 Hz, 1H), 1.52 (s, 3H), 1.42 (s, 3H)

¹³C NMR (101 MHz, CDCl₃): δ 166.25, 161.06, 156.26, 154.20, 143.02, 134.53, 131.59, 130.51, 130.31, 130.22, 129.23, 127.20, 115.85, 113.72, 113.33, 105.12, 77.52, 48.96, 28.74, 24.93, 24.78.

MS (ESI): m/z [M+H]⁺ 384.1, found.

### Example 28: Preparation of N-(8,8-dimethyl-2-oxo-7,8-dihydro-2H,6H-pyrano[3,2-g]chromen-7-yl)-2-hydroxybenzamide (Compound 29)

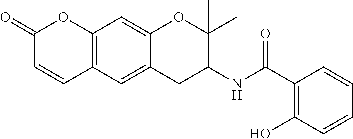 [SMILES: CC1(C)Oc2cc3oc(=O)ccc3cc2CC1NC(=O)c1ccccc1O]

Compound 1 (70 mg, 0.29 mmol) and 2-hydroxybenzoic acid (48 mg, 0.35 mmol) were reacted according to the method described in Preparation Example 2. The dichloromethane extract was concentrated, and the residue was purified by silica gel column chromatography using a mixture of dichloromethane and methanol (v/v, 100:1), affording Compound 29 as a white solid (56 mg, 53%).

Mp: 194° C.

¹H NMR (400 MHz, CDCl₃): δ 12.06 (s, 1H), 7.58 (d, J=9.5 Hz, 1H), 7.38 (ddd, J=8.6, 7.3, 1.5 Hz, 1H), 7.25-7.17 (m, 2H), 6.97 (dd, J=8.4, 1.0 Hz, 1H), 6.85 (s, 1H), 6.82-6.75 (m, 1H), 6.45 (d, J=8.8 Hz, 1H), 6.24 (d, J=9.5 Hz, 1H), 4.51 (ddd, J=8.6, 5.1, 2.9 Hz, 1H), 3.28 (ddd, J=17.1, 5.1, 0.9 Hz, 1H), 2.93 (dd, J=17.2, 2.8 Hz, 1H), 1.49 (s, 3H), 1.41 (s, 3H).

¹³C NMR (101 MHz, CDCl₃): δ 169.75, 161.56, 160.99, 156.10, 154.22, 142.97, 134.60, 129.40, 125.37, 118.80, 118.69, 115.57, 113.89, 113.86, 113.56, 105.31, 77.47, 48.15, 28.69, 24.98, 24.74.

MS (ESI): m/z [M+H]⁺ 366.1, found.

### Example 29: Preparation of 2-((8,8-dimethyl-2-oxo-7,8-dihydro-2H,6H-pyrano[3,2-g]chromen-7-yl)carbamoyl)phenyl acetate (Compound 30)

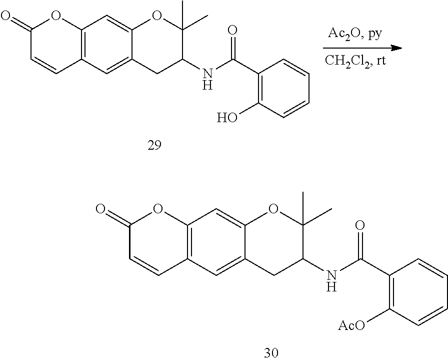 [SMILES: CC1(C)Oc2cc3oc(=O)ccc3cc2CC1NC(=O)c1ccccc1O ; CC(=O)Oc1ccccc1C(=O)NC1Cc2cc3ccc(=O)oc3cc2OC1(C)C]

Compound 29 (50 mg, 0.14 mmol), acetic anhydride (26 μL, 0.28 mmol), and pyridine (23 μL, 0.28 mmol) were dissolved in dichloromethane, and the resulting solution was stirred at room temperature for 1 hour. Upon completion of the reaction, the reaction mixture was quenched by addition of an aqueous 1 N hydrochloric acid solution and extracted with dichloromethane. The dichloromethane extract was dried over anhydrous sodium sulfate, concentrated, and the concentrate was purified and separated by silica gel column chromatography using a mixture of dichloromethane and methanol (v/v, 100:1), thereby affording Compound 30 as a white solid (21 mg, 37%).

Mp: 158-160° C.

¹H NMR (400 MHz, CDCl₃): δ 7.88 (dd, J=7.8, 1.6 Hz, 1H), 7.58 (d, J=9.5 Hz, 1H), 7.50-7.41 (m, 1H), 7.30 (t, J=7.2 Hz, 1H), 7.21 (s, 1H), 7.06 (d, J=7.7 Hz, 1H), 6.82 (s, 1H), 6.73 (d, J=8.9 Hz, 1H), 6.25 (d, J=9.5 Hz, 1H), 4.54 (ddd, J=8.8, 5.1, 2.6 Hz, 1H), 3.27 (dd, J=17.2, 4.8 Hz, 1H), 2.87 (dd, J=17.2, 2.3 Hz, 1H), 2.14 (s, 3H), 1.48 (s, 3H), 1.39 (s, 3H).

¹³C NMR (101 MHz, CDCl₃): δ 168.68, 164.90, 160.95, 156.21, 154.21, 147.93, 142.95, 132.26, 130.52, 129.52, 126.83, 126.34, 123.21, 115.78, 113.91, 113.46, 105.10, 77.74, 48.26, 28.80, 25.03, 24.68, 20.72.

MS (ESI): m/z [M+H]⁺ 408.1, found.

### Example 30: Preparation of 2-bromo-N-(8,8-dimethyl-2-oxo-7,8-dihydro-2H,6H-pyrano[3,2-g]chromen-7-yl)acetamide (Compound 31)

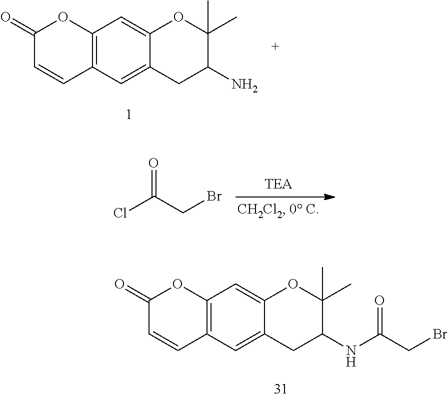 [SMILES: CC1(C)Oc2cc3oc(=O)ccc3cc2CC1N ; O=C(Cl)CBr ; CC1(C)Oc2cc3oc(=O)ccc3cc2CC1NC(=O)CBr]

Compound 1 (100 mg, 0.41 mmol) and triethylamine (TEA) (68 μL, 0.49 mmol) were dissolved in dichloromethane, and the resulting solution was stirred at 0° C. for 10 minutes. After 10 minutes, 2-bromoacetyl chloride (37 μL, 0.45 mmol) was slowly added, and the mixture was stirred for 1 hour. Upon completion of the reaction, the reaction mixture was quenched by addition of a saturated aqueous ammonium chloride solution and extracted with dichloromethane. The dichloromethane extract was dried over anhydrous sodium sulfate, concentrated, and the concentrate was purified and separated by silica gel column chromatography using a mixture of dichloromethane and methanol (v/v, 150:1), thereby affording Compound 31 as a white solid (134 mg, 89%).

Mp: 235° C.

¹H NMR (400 MHz, CDCl₃): δ 7.58 (d, J=9.5 Hz, 1H), 7.19 (s, 1H), 6.78 (s, 1H), 6.63 (d, J=8.6 Hz, 1H), 6.23 (d, J=9.5 Hz, 1H), 4.28 (ddd, J=8.8, 5.1, 3.5 Hz, 1H), 3.85 (s, 2H), 3.19 (dd, J=17.1, 4.5 Hz, 1H), 2.81 (dd, J=17.1, 3.3 Hz, 1H), 1.39 (d, J=26.4 Hz, 6H).

¹³C NMR (101 MHz, CDCl₃): δ 165.53, 161.04, 156.16, 154.20, 142.99, 129.20, 115.52), 113.77, 113.39, 105.19, 48.96, 28.91, 28.50, 24.81, 24.47.

MS (ESI): m/z [M+H]⁺ 366.0, found.

### Example 31: Preparation of N-(8,8-dimethyl-2-oxo-7,8-dihydro-2H,6H-pyrano[3,2-g]chromen-7-yl)-2-(piperazin-1-yl)acetamide (Compound 32)

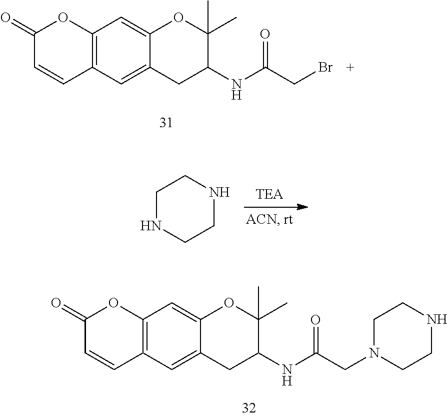 [SMILES: CC1(C)Oc2cc3oc(=O)ccc3cc2CC1NC(=O)CBr ; C1CNCCN1 ; CC1(C)Oc2cc3oc(=O)ccc3cc2CC1NC(=O)CN1CCNCC1]

Piperazine (24 mg, 0.28 mmol) and triethylamine (TEA) (49 μL, 0.35 mmol) were dissolved in acetonitrile, and the resulting solution was stirred at room temperature for 10 minutes. After 10 minutes, Compound 31 (50 mg, 0.14 mmol) was added, and the mixture was stirred for 4 hours. Upon completion of the reaction, the reaction mixture was quenched by addition of distilled water and extracted with ethyl acetate. The ethyl acetate extract was dried over anhydrous sodium sulfate, concentrated, and the concentrate was purified and separated by silica gel column chromatography using a mixture of dichloromethane and methanol (v/v, 50:1), thereby affording Compound 32 as a white solid (45 mg, 87%).

Mp: 279° C.

¹H NMR (400 MHz, CDCl₃): δ 7.59 (d, J=9.5 Hz, 1H), 7.41 (d, J=9.0 Hz, 1H), 7.18 (s, 1H), 6.83 (s, 1H), 6.25 (d, J=9.5 Hz, 1H), 4.24 (dt, J=8.8, 4.2 Hz, 1H), 3.19-3.12 (m, 1H), 3.05-2.90 (m, 2H), 2.82 (dd, J=16.9, 3.6 Hz, 1H), 2.74 (ddt, J=14.4, 7.8, 2.9 Hz, 4H), 2.50-2.33 (m, 6H), 1.40 (s, 3H), 1.36 (s, 3H).

¹³C NMR (101 MHz, CDCl₃) δ 169.92, 161.09, 156.35, 154.18, 143.02, 129.17, 115.93, 113.73, 113.19, 104.92, 61.70, 53.80, 47.83, 45.61, 28.69, 24.95, 24.66.

MS (ESI): m/z [M+H]⁺ 372.2, found.

### Example 32: Preparation of N-(8,8-dimethyl-2-oxo-7,8-dihydro-2H,6H-pyrano[3,2-g]chromen-7-yl)-2-(4-isopropylpiperazin-1-yl)acetamide (Compound 33)

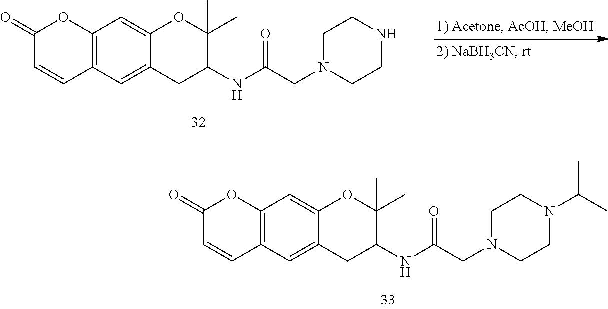 [SMILES: CC1(C)Oc2cc3oc(=O)ccc3cc2CC1NC(=O)CN1CCNCC1 ; CC(C)N1CCN(CC(=O)NC2Cc3cc4ccc(=O)oc4cc3OC2(C)C)CC1]

Acetic acid (9 μL, 0.16 mmol) was added to a solution of Compound 32 (50 mg, 0.13 mmol) and acetone (10 μL, 0.14 mmol) in methanol, and the mixture was stirred at room temperature for 15 minutes. After 15 minutes, sodium cyanoborohydride (8 mg, 0.13 mmol) was added, and the mixture was stirred for 24 hours. Upon completion of the reaction, the reaction mixture was quenched by addition of distilled water and concentrated. The concentrate was diluted with dichloromethane and washed with distilled water. The dichloromethane extract was dried over anhydrous sodium sulfate, concentrated, and the concentrate was purified and separated by silica gel column chromatography using a mixture of dichloromethane and methanol (v/v, 50:1), thereby affording Compound 33 as a white solid (24 mg, 45%).

Mp: 132° C.

¹H NMR (400 MHz, CDCl₃): δ 7.59 (d, J=9.5 Hz, 1H), 7.51 (d, J=9.3 Hz, 1H), 7.18 (s, 1H), 6.84 (s, 1H), 6.25 (d, J=9.5 Hz, 1H), 4.28-4.20 (m, 1H), 3.21-3.11 (m, 1H), 2.96 (dd, J=41.4, 16.4 Hz, 2H), 2.79 (dd, J=16.9, 3.6 Hz, 1H), 2.56 (dt, J=13.0, 6.5 Hz, 1H), 2.47 (d, J=2.9 Hz, 2H), 2.40-2.28 (m, 4H), 1.94 (s, 2H), 1.39 (d, J=13.1 Hz, 6H), 0.94 (dd, J=6.5, 3.6 Hz, 6H).

¹³C NMR (101 MHz, CDCl₃): 170.19, 161.02, 156.47, 154.26, 142.98, 129.22, 115.94, 113.64, 113.13, 104.87, 77.56, 61.03, 54.32, 53.46, 48.51, 47.70, 28.84, 25.06, 24.71, 18.26, 18.21.

MS (ESI): m/z [M+H]⁺ 414.2, found.

### Example 33: Preparation of N-(8,8-dimethyl-2-oxo-7,8-dihydro-2H,6H-pyrano[3,2-g]chromen-7-yl)-2-morpholinoacetamide (Compound 34)

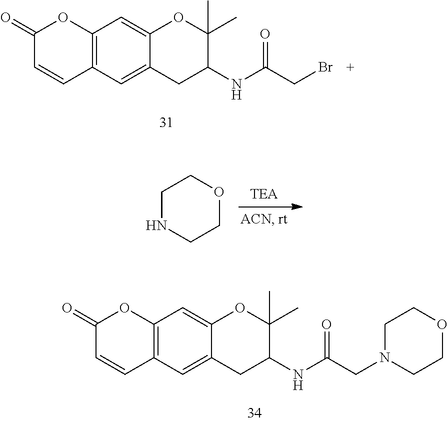 [SMILES: CC1(C)Oc2cc3oc(=O)ccc3cc2CC1NC(=O)CBr ; C1COCCN1 ; CC1(C)Oc2cc3oc(=O)ccc3cc2CC1NC(=O)CN1CCOCC1]

A solution of morpholine (24 μL, 0.28 mmol) and triethylamine (TEA) (49 μL, 0.35 mmol) in acetonitrile was stirred at room temperature for 10 minutes. After 10 minutes, Compound 31 (50 mg, 0.14 mmol) was added, and the mixture was stirred for 3 hours. Upon completion of the reaction, the reaction mixture was quenched by addition of distilled water and extracted with ethyl acetate. The ethyl acetate extract was dried over anhydrous sodium sulfate, concentrated, and the concentrate was purified and separated by silica gel column chromatography using a mixture of dichloromethane and methanol (v/v, 100:1), thereby affording Compound 34 as a white solid (42 mg, 80%).

Mp: 183° C.

¹H NMR (400 MHz, CDCl₃): δ 7.58 (d, J=9.5 Hz, 1H), 7.38 (d, J=9.0 Hz, 1H), 7.17 (s, 1H), 6.84 (s, 1H), 6.26 (d, J=9.5 Hz, 1H), 4.24 (ddd, J=8.8, 4.7, 3.8 Hz, 1H), 3.56-3.41 (m, 4H), 3.16 (ddd, J=16.8, 4.8, 0.8 Hz, 1H), 2.98 (dd, J=36.1, 16.4 Hz, 2H), 2.81 (dd, J=16.9, 3.6 Hz, 1H), 2.50-2.29 (m, 4H), 1.40 (s, 3H), 1.36 (s, 3H).

¹³C NMR (101 MHz, CDCl₃): 169.72, 161.00, 156.32, 154.21, 142.91, 129.12, 115.85, 113.82, 113.21, 104.93, 66.79, 61.69, 53.68, 47.85, 28.72, 24.93, 24.65.

MS (ESI): m/z [M+H]⁺ 373.2, found.

### Example 34: Preparation of N-(8,8-dimethyl-2-oxo-7,8-dihydro-2H,6H-pyrano[3,2-g]chromen-7-yl)-2-(4-(2-hydroxyethyl)piperidin-1-yl)acetamide (Compound 35)

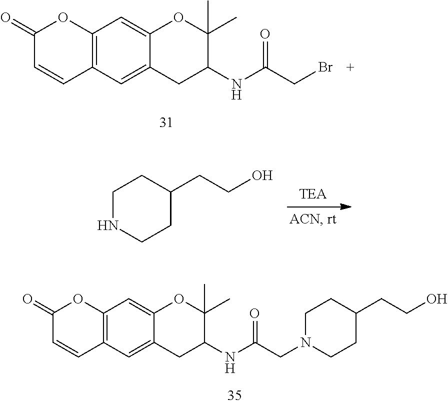 [SMILES: CC1(C)Oc2cc3oc(=O)ccc3cc2CC1NC(=O)CBr ; OCCC1CCNCC1 ; CC1(C)Oc2cc3oc(=O)ccc3cc2CC1NC(=O)CN1CCC(CCO)CC1]

A solution of 4-piperidineethanol (24 μL, 0.28 mmol) and triethylamine (TEA) (49 μL, 0.35 mmol) in acetonitrile was stirred at room temperature for 10 minutes. After 10 minutes, Compound 31 (50 mg, 0.14 mmol) was added, and the mixture was stirred for 3 hours. Upon completion of the reaction, the reaction mixture was quenched by addition of distilled water and extracted with ethyl acetate. The ethyl acetate extract was dried over anhydrous sodium sulfate, concentrated, and the concentrate was purified and separated by silica gel column chromatography using a mixture of dichloromethane and methanol (v/v, 100:1), thereby affording Compound 35 as a white solid (29 mg, 50%).

Mp: 86° C.

¹H NMR (400 MHz, CDCl₃): δ 7.58 (dd, J=13.7, 9.4 Hz, 2H), 7.18 (s, 1H), 6.83 (s, 1H), 6.25 (d, J=9.5 Hz, 1H), 4.23 (dt, J=9.0, 4.3 Hz, 1H), 3.65-3.55 (m, 2H), 3.15 (dd, J=16.8, 4.7 Hz, 1H), 2.92 (q, J=16.4 Hz, 2H), 2.83-2.71 (m, 2H), 2.47 (dd, J=7.2, 4.3 Hz, 1H), 2.07 (dtd, J=34.3, 11.7, 2.5 Hz, 2H), 1.70-1.60 (m, 2H), 1.46-1.40 (m, 2H), 1.37 (t, J=8.0 Hz, 8H), 1.07 (ddd, J=15.7, 12.5, 3.9 Hz, 1H), 0.70 (ddd, J=15.8, 12.5, 3.8 Hz, 1H).

¹³C NMR (101 MHz, CDCl₃): δ 170.64, 161.16, 156.48, 154.22, 143.08, 129.21, 116.09, 113.56, 113.13, 104.88, 77.53, 61.59, 60.25, 54.17, 54.02, 47.76, 39.13, 32.60, 32.36, 31.51, 28.79, 25.09, 24.50.

MS (ESI): m/z [M+H]⁺ 415.2, found.

### Example 35: Preparation of (E)-4-(3-(4-(2-((8,8-dimethyl-2-oxo-7,8-dihydro-2H,6H-pyrano[3,2-g]chromen-7-yl)amino)-2-oxoethyl)piperazin-1-yl)-3-oxoprop-1-en-1-yl)-2-methoxyphenyl acetate (Compound 36)

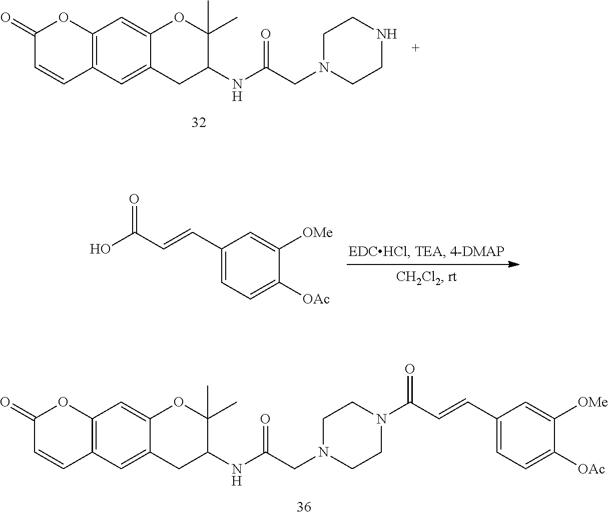 [SMILES: CC1(C)Oc2cc3oc(=O)ccc3cc2CC1NC(=O)CN1CCNCC1 ; COc1cc(/C=C/C(=O)O)ccc1OC(C)=O ; COc1cc(/C=C/C(=O)N2CCN(CC(=O)NC3Cc4cc5ccc(=O)oc5cc4OC3(C)C)CC2)ccc1OC(C)=O]

A solution of Compound 32 (150 mg, 0.40 mmol), (E)-3-(4-acetoxy-3-methoxyphenyl)acrylic acid (113 mg, 0.48 mmol), EDC·HCl (115 mg, 0.60 mmol), triethylamine (TEA) (84 μL, 0.60 mmol), and 4-dimethylaminopyridine (4-DMAP) (39 mg, 0.32 mmol) in dichloromethane was stirred at room temperature for 18 hours. Upon completion of the reaction, the reaction mixture was quenched by addition of a saturated aqueous ammonium chloride solution and extracted with dichloromethane. The dichloromethane extract was dried over anhydrous sodium sulfate, concentrated, and the concentrate was purified and separated by silica gel column chromatography using a mixture of dichloromethane and methanol (v/v, 100:1), thereby affording Compound 36 as a white solid (212 mg, 90%).

Mp: 160° C.

¹H NMR (400 MHz, CDCl₃): δ 7.61-7.55 (m, 2H), 7.30 (d, J=9.1 Hz, 1H), 7.20-7.03 (m, 4H), 6.84 (s, 1H), 6.70 (d, J=15.4 Hz, 1H), 6.25 (d, J=9.5 Hz, 1H), 4.30-4.23 (m, 1H), 3.86 (s, 3H), 3.64 (b, 1H), 3.47 (b, 3H), 3.18 (dd, J=17.2, 4.6 Hz, 1H), 3.03 (q, J=16.3 Hz, 2H), 2.82 (dd, J=17.0, 3.3 Hz, 1H), 2.50 (b, 2H), 2.45-2.36 (m, 2H), 2.32 (s, 3H), 1.41 (s, 3H), 1.37 (s, 3H).

¹³C NMR (101 MHz, CDCl₃): δ 169.39, 168.85, 165.31, 160.93, 156.27, 154.22, 151.34, 142.91, 142.58, 140.97, 134.13, 129.18, 123.19, 120.29, 117.03, 115.76, 113.92, 113.31, 111.68, 104.97, 61.24, 55.99, 47.87, 28.75, 24.86, 24.80, 20.66.

MS (ESI): m/z [M+H]⁺ 590.2, found.

### Example 36: Preparation of (E)-N-(8,8-dimethyl-2-oxo-7,8-dihydro-2H,6H-pyrano[3,2-g]chromen-7-yl)-2-(4-(3-(4-hydroxy-3-methoxyphenyl)acryloyl)piperazin-1-yl)acetamide (Compound 37)

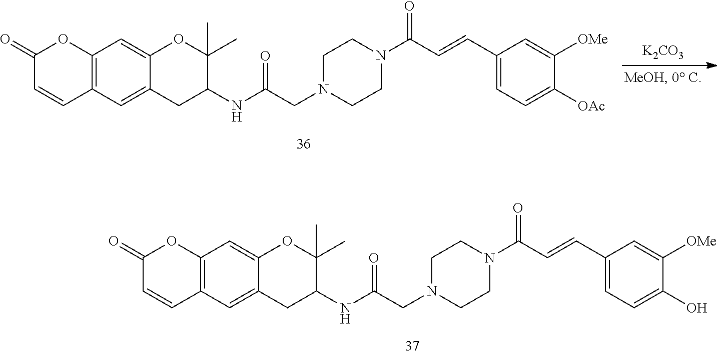

Compound 36 (164 mg, 0.28 mmol) was dissolved in dichloromethane, and the solution was cooled to 0° C. for 10 minutes. Potassium carbonate (43 mg, 0.31 mmol) was then added, and the mixture was stirred for 1 hour. After completion of the reaction, 1 N aqueous hydrochloric acid was added to quench the reaction, and the mixture was extracted with dichloromethane. The dichloromethane extract was dried over anhydrous sodium sulfate and concentrated, and the concentrate was purified and separated by silica gel column chromatography using a mixture of dichloromethane and methanol (v/v, 50:1), affording Compound 37 as a white solid (136 mg, 89%).

Mp: 163-165° C.

¹H NMR (400 MHz, CDCl₃): δ 7.58 (dd, J=12.4, 9.8 Hz, 2H), 7.32 (d, J=9.1 Hz, 1H), 7.19 (s, 1H), 7.07 (dd, J=8.2, 1.8 Hz, 1H), 6.96 (d, J=1.8 Hz, 1H), 6.90 (d, J=8.2 Hz, 1H), 6.83 (s, 1H), 6.61 (d, J=15.3 Hz, 1H), 6.25 (d, J=9.5 Hz, 1H), 5.91 (s, 1H), 4.27 (ddd, J=8.6, 4.6, 3.7 Hz, 1H), 3.92 (s, 3H), 3.70-3.57 (m, 1H), 3.48 (b, 3H), 3.18 (dd, J=17.1, 4.4 Hz, 1H), 3.03 (q, J=16.3 Hz, 2H), 2.83 (dd, J=17.0, 3.4 Hz, 1H), 2.49 (b, 2H), 2.44-2.35 (m, 2H), 1.41 (s, 3H), 1.37 (s, 3H).

¹³C NMR (101 MHz, CDCl₃): δ 169.45, 165.77, 160.94, 156.27, 154.22, 147.48, 146.71, 143.45, 142.92, 129.17, 127.66, 121.89, 115.77, 114.77, 114.06, 113.90, 113.30, 110.00, 104.96, 61.25, 56.02, 47.88, 28.74, 24.87, 24.79.

MS (ESI): m/z [M+H]⁺ 548.2, found.

### Example 37: Preparation of N-(8,8-dimethyl-2-oxo-7,8-dihydro-2H,6H-pyrano[3,2-g]chromen-7-yl)-8-((1,4-dioxo-1,4-dihydronaphthalen-2-yl)thio)octanamide (Compound 38)

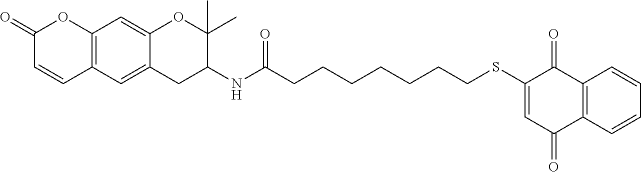 [SMILES: CC1(C)Oc2cc3oc(=O)ccc3cc2CC1NC(=O)CCCCCCCSC1=CC(=O)c2ccccc2C1=O]

Compound 1 (66 mg, 0.27 mmol), 8-((1,4-dioxo-1,4-dihydronaphthalen-2-yl)thio)octanoic acid (90 mg, 0.27 mmol), EDC·HCl (104 mg, 0.54 mmol), hydroxybenzotriazole (55 mg, 0.41 mmol), and 4-methylmorpholine (59 μL, 0.54 mmol) were dissolved in N,N-dimethylformamide (DMF), and the resulting solution was stirred at room temperature for 16 hours. Upon completion of the reaction, the precipitate was vacuum filtered, washed with distilled water, and dried, thereby affording Compound 38 as a yellow solid (127 mg, 84%).

Mp: 221° C.

¹H NMR (400 MHz, CDCl₃): δ 8.13-8.04 (m, 2H), 7.79-7.68 (m, 2H), 7.58 (d, J=9.5 Hz, 1H), 7.18 (s, 1H), 6.74 (s, 1H), 6.57 (s, 1H), 6.21 (d, J=9.5 Hz, 1H), 5.79 (d, J=9.2 Hz, 1H), 4.34 (dq, J=8.6, 3.0 Hz, 1H), 3.18 (dd, J=17.3, 4.8 Hz, 1H), 2.85-2.72 (m, 3H), 2.23-2.12 (m, 2H), 1.73 (p, J=7.4 Hz, 2H), 1.63 (p, J=7.4 Hz, 2H), 1.51-1.43 (m, 2H), 1.41 (s, 3H), 1.38-1.27 (m, 7H).

¹³C NMR (101 MHz, CDCl₃): 182.13, 181.55, 172.85, 161.07, 156.39, 155.22, 154.10, 143.06, 134.35, 133.30, 132.18, 131.92, 129.30, 126.96, 126.89, 126.54, 115.99, 113.60, 113.24, 105.08, 77.64, 47.68, 36.62, 30.54, 28.96, 28.87, 28.77, 28.70, 27.17, 25.55, 24.87, 24.78.

MS (ESI): m/z [M+H]⁺ 560.2, found.

### Example 38: Preparation of N-(8,8-dimethyl-2-oxo-7,8-dihydro-2H,6H-pyrano[3,2-g]chromen-7-yl)-3-((3-hydroxy-1,4-dioxo-1,4-dihydronaphthalen-2-yl)thio)propanamide (Compound 39)

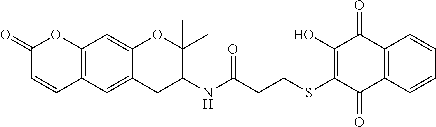 [SMILES: CC1(C)Oc2cc3oc(=O)ccc3cc2CC1NC(=O)CCSC1=C(O)C(=O)c2ccccc2C1=O]

Compound 1 (88 mg, 0.36 mmol), 3-((3-hydroxy-1,4-dioxo-1,4-dihydronaphthalen-2-yl)thio)propanoic acid (100 mg, 0.36 mmol), EDC·HCl (138 mg, 0.72 mmol), hydroxybenzotriazole (73 mg, 0.54 mmol), and 4-methylmorpholine (79 μL, 0.72 mmol) were dissolved in N,N-dimethylformamide (DMF), and the mixture was stirred at room temperature for 16 hours. Upon completion of the reaction, 1 N aqueous hydrochloric acid was added to quench the reaction, and the mixture was extracted with dichloromethane. The dichloromethane extract was washed several times with distilled water and brine, dried over anhydrous sodium sulfate, and concentrated. The concentrate was purified and separated by silica gel column chromatography using a mixture of dichloromethane and methanol (v/v, 50:1), thereby affording Compound 39 as a brown solid (105 mg, 58%).

Mp: 233° C.

¹H NMR (400 MHz, DMSO): δ 8.59 (d, J=8.4 Hz, 1H), 8.05-7.87 (m, 3H), 7.82 (d, J=7.2 Hz, 1H), 7.69 (t, J=7.3 Hz, 1H), 7.57 (t, J=7.1 Hz, 1H), 7.47 (s, 1H), 6.77 (s, 1H), 6.25 (d, J=9.3 Hz, 1H), 4.14 (q, J=8.1 Hz, 1H), 3.01-2.77 (m, 4H), 2.31-2.17 (m, 2H), 1.37 (s, 3H), 1.31 (s, 3H).

¹³C NMR (101 MHz, DMSO): δ 185.58, 179.5, 172.71, 171.80, 160.78, 156.71, 154.02, 144.66, 136.32, 134.24, 131.63, 131.12, 129.75, 126.13, 125.83, 124.37, 118.48, 113.02, 112.87, 104.01, 79.08, 48.67, 36.54, 29.85, 28.45, 26.53, 21.46.

MS (ESI): m/z [M+H]⁺ 506.1, found.

### Example 39: Preparation of N-(8,8-dimethyl-2-oxo-7,8-dihydro-2H,6H-pyrano[3,2-g]chromen-7-yl)-3-((3-methyl-1,4-dioxo-1,4-dihydronaphthalen-2-yl)thio)propanamide (Compound 40)

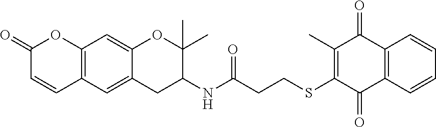 [SMILES: CC1=C(SCCC(=O)NC2Cc3cc4ccc(=O)oc4cc3OC2(C)C)C(=O)c2ccccc2C1=O]

Compound 1 (50 mg, 0.20 mmol), 3-((3-methyl-1,4-dioxo-1,4-dihydronaphthalen-2-yl)thio)propanoic acid (66 mg, 0.24 mmol), and EDC·HCl (77 mg, 0.40 mmol) were dissolved in dichloromethane, and the mixture was stirred at room temperature for 16 hours. Upon completion of the reaction, distilled water was added to quench the reaction, and the mixture was extracted with dichloromethane. The dichloromethane extract was dried over anhydrous sodium sulfate and concentrated, and the concentrate was purified and separated by silica gel column chromatography using a mixture of dichloromethane and methanol (v/v, 100:1), thereby affording Compound 40 as a yellow solid (93 mg, 92%).

Mp: 209° C.

¹H NMR (400 MHz, DMSO): δ 8.06-7.88 (m, 4H), 7.83 (dq, J=7.3, 4.0 Hz, 2H), 7.42 (s, 1H), 6.75 (s, 1H), 6.26 (d, J=9.5 Hz, 1H), 2.95 (dd, J=17.0, 5.5 Hz, 1H), 2.63 (dd, J=16.9, 7.3 Hz, 1H), 2.48 (dd, J=6.8, 2.1 Hz, 2H), 2.21 (s, 3H), 1.27 (s, 3H), 1.21 (s, 3H).

¹³C NMR (101 MHz, DMSO): δ 182.20, 181.05, 170.64, 160.73, 156.57, 153.98, 147.26, 145.99, 144.59, 134.43, 134.24, 133.05, 132.05, 129.86, 126.93, 126.53, 117.80, 113.11, 112.97, 104.09, 78.55, 48.27, 36.47, 30.04, 28.32, 26.01, 22.46, 15.62.

MS (ESI): m/z [M+H]⁺ 504.1, found.

### Example 40: Preparation of N-(8,8-dimethyl-2-oxo-7,8-dihydro-2H,6H-pyrano[3,2-g]chromen-7-yl)-3-((1,4-dioxo-1,4-dihydronaphthalen-2-yl)sulfinyl)propanamide (Compound 41)

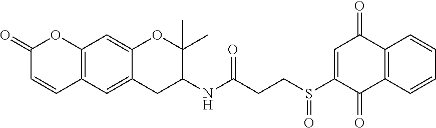 [SMILES: CC1(C)Oc2cc3oc(=O)ccc3cc2CC1NC(=O)CCS(=O)C1=CC(=O)c2ccccc2C1=O]

Compound 1 (50 mg, 0.20 mmol), 3-((1,4-dioxo-1,4-dihydronaphthalen-2-yl)sulfinyl)propanoic acid (67 mg, 0.24 mmol), EDC·HCl (77 mg, 0.40 mmol), hydroxybenzotriazole (41 mg, 0.54 mmol), and 4-methylmorpholine (44 μL, 0.40 mmol) were dissolved in N,N-dimethylformamide (DMF), and the mixture was stirred at room temperature for 16 hours. Upon completion of the reaction, the reaction mixture was quenched by addition of an aqueous 1 N hydrochloric acid solution and extracted with dichloromethane. The dichloromethane extract was washed several times with distilled water and brine, dried over anhydrous sodium sulfate, and concentrated. The concentrate was purified and separated by silica gel column chromatography using a mixture of dichloromethane and methanol (v/v, 50:1), thereby affording Compound 41 as a yellow solid (86 mg, 85%).

Mp: 168° C.

¹H NMR (400 MHz, DMSO): δ 8.11 (ddd, J=6.9, 4.4, 2.0 Hz, 2H), 7.84-7.76 (m, 2H), 7.53 (dd, J=12.2, 9.5 Hz, 1H), 7.23 (s, 1H), 7.14 (d, J=12.4 Hz, 1H), 6.67 (s, 1H), 6.43 (dd, J=22.9, 9.1 Hz, 1H), 6.13 (dd, J=9.5, 2.8 Hz, 1H), 4.12 (dddd, J=23.9, 8.7, 5.1, 3.2 Hz, 1H), 3.58-3.33 (m, 2H), 3.14-2.97 (m, 1H), 2.76-2.47 (m, 3H), 1.33 (d, J=11.2 Hz, 3H), 1.26 (s, 3H).

MS (ESI): m/z [M+H]⁺ 506.1, found.

### Example 41: Preparation of N-(8,8-dimethyl-2-oxo-7,8-dihydro-2H,6H-pyrano[3,2-g]chromen-7-yl)-2-(1-(((1,4-dioxo-1,4-dihydronaphthalen-2-yl)thio)methyl)cyclopropyl)acetamide (Compound 42)

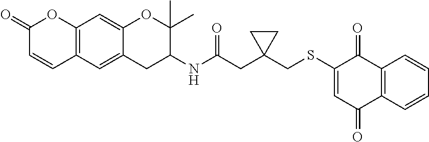 [SMILES: CC1(C)Oc2cc3oc(=O)ccc3cc2CC1NC(=O)CC1(CSC2=CC(=O)c3ccccc3C2=O)CC1]

Compound 1 (60 mg, 0.24 mmol), 2-(1-(((1,4-dioxo-1,4-dihydronaphthalen-2-yl)thio)methyl)cyclopropyl)acetic acid (89 mg, 0.31 mmol), and EDC·HCl (92 mg, 0.48 mmol) were dissolved in dichloromethane, and the mixture was stirred at room temperature for 16 hours. Upon completion of the reaction, the reaction mixture was quenched by addition of distilled water and extracted with dichloromethane. The dichloromethane extract was dried over anhydrous sodium sulfate and concentrated, and the concentrate was purified and separated by silica gel column chromatography using a mixture of dichloromethane and methanol (v/v, 100:1), thereby affording Compound 42 as a yellow solid (114 mg, 90%).

Mp: 220° C.

¹H NMR (400 MHz, DMSO): δ 8.10-8.03 (m, 2H), 7.74 (dtd, J=18.6, 7.4, 1.5 Hz, 2H), 7.46 (d, J=9.4 Hz, 1H), 7.13 (s, 1H), 6.66 (s, 1H), 6.47 (s, 1H), 6.09 (d, J=9.5 Hz, 1H), 6.00 (d, J=9.0 Hz, 1H), 4.32 (ddt, J=8.1, 5.0, 2.7 Hz, 1H), 3.21-3.12 (m, 1H), 2.95 (d, J=12.0 Hz, 1H), 2.83-2.75 (m, 2H), 2.41 (d, J=15.0 Hz, 1H), 2.24 (d, J=15.0 Hz, 1H), 1.40 (s, 3H), 1.31 (s, 3H), 0.77-0.61 (m, 4H).

¹³C NMR (101 MHz, DMSO): δ 181.77, 181.37, 170.52, 160.90, 156.26, 154.72, 154.01, 142.86, 134.48, 133.47, 132.05, 131.69, 129.24, 127.32, 126.91, 126.60, 115.79, 113.58, 113.20, 105.09, 77.38, 47.96, 42.47, 39.17, 28.85, 24.96, 24.68, 16.19, 13.17, 13.10.

MS (ESI): m/z [M+H]⁺ 530.2, found.

### Example 42: Preparation of N-(8,8-dimethyl-2-oxo-7,8-dihydro-2H,6H-pyrano[3,2-g]chromen-7-yl)-2-(4-(3-((1,4-dioxo-1,4-dihydronaphthalen-2-yl)thio)propanoyl)piperazin-1-yl)acetamide (Compound 43)

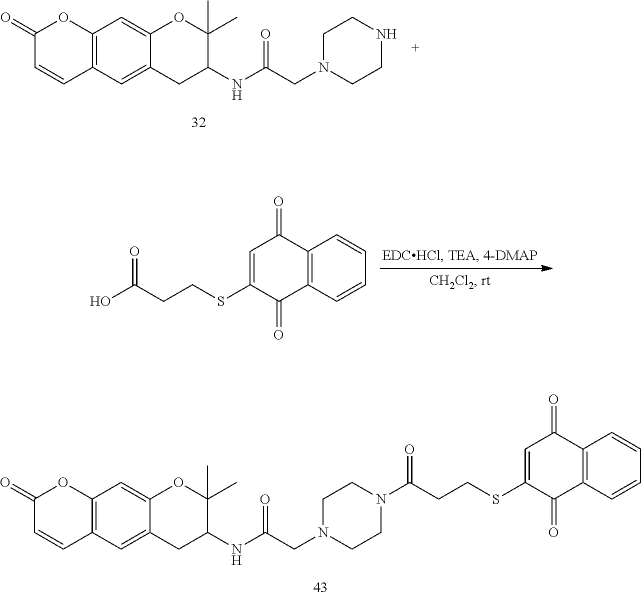 [SMILES: CC1(C)Oc2cc3oc(=O)ccc3cc2CC1NC(=O)CN1CCNCC1 ; O=C(O)CCSC1=CC(=O)c2ccccc2C1=O ; CC1(C)Oc2cc3oc(=O)ccc3cc2CC1NC(=O)CN1CCN(C(=O)CCSC2=CC(=O)c3ccccc3C2=O)CC1]

Compound 32 (60 mg, 0.16 mmol), 3-((1,4-dioxo-1,4-dihydronaphthalen-2-yl)thio)propanoic acid (42 mg, 0.16 mmol), EDC·HCl (61 mg, 0.32 mmol), hydroxybenzotriazole (32 mg, 0.24 mmol), and N,N-diisopropylethylamine (56 μL, 0.32 mmol) were dissolved in N,N-dimethylformamide (DMF), and the mixture was stirred at room temperature for 16 hours. Upon completion of the reaction, the reaction mixture was quenched by addition of an aqueous 1 N hydrochloric acid solution and extracted with dichloromethane. The dichloromethane extract was washed several times with distilled water and brine, dried over anhydrous sodium sulfate, and concentrated. The concentrate was purified and separated by silica gel column chromatography using a mixture of dichloromethane and methanol (v/v, 100:1), thereby affording Compound 43 as a yellow solid (56 mg, 57%).

Mp: 178-180° C.

¹H NMR (400 MHz, CDCl₃): δ 8.12-8.05 (m, 2H), 7.73 (dtd, J=19.4, 7.4, 1.3 Hz, 2H), 7.58 (d, J=9.5 Hz, 1H), 7.24 (d, J=9.1 Hz, 1H), 7.18 (s, 1H), 6.82 (s, 1H), 6.65 (s, 1H), 6.26 (d, J=9.5 Hz, 1H), 4.31-4.22 (m, 1H), 3.60-3.51 (m, 1H), 3.40-3.32 (m, 1H), 3.29 (t, J=4.7 Hz, 2H), 3.21-3.12 (m, 3H), 3.02 (q, J=16.3 Hz, 2H), 2.81 (dd, J=17.0, 3.2 Hz, 1H), 2.69 (t, J=7.2 Hz, 2H), 2.54-2.41 (m, 2H), 2.37 (dt, J=10.6, 4.8 Hz, 2H), 1.40 (s, 3H), 1.36 (s, 3H).

¹³C NMR (101 MHz, CDCl₃): δ 182.01, 181.49, 169.27, 168.19, 160.90, 156.25, 154.45, 154.21, 142.89, 134.41, 133.35, 132.16, 131.87, 129.16, 127.07, 126.88, 126.59, 115.74, 113.93, 113.30, 104.97, 61.22, 53.28, 52.96, 47.85, 45.10, 41.67, 30.99, 28.76, 25.49, 24.85, 24.78.

MS (ESI): m/z [M+H]⁺ 616.2, found.

### Example 43: Preparation of N-(2-(2-(2-((2-((8,8-dimethyl-2-oxo-7,8-dihydro-2H,6H-pyrano[3,2-g]chromen-7-yl)amino)-2-oxoethyl)amino)ethoxy)ethoxy)ethyl)-3-((1,4-dioxo-1,4-dihydronaphthalen-2-yl)thio)propanamide (Compound 44)

1) Preparation of tert-butyl (2-(2-(2-((2-((8,8-dimethyl-2-oxo-7,8-dihydro-2H,6H-pyrano[3,2-g]chromen-7-yl)amino)-2-oxoethyl)amino)ethoxy)ethoxy)ethyl)carbamate (Compound 44a)

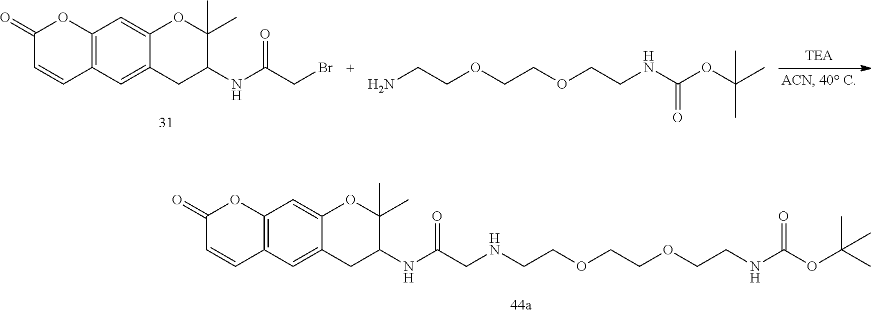 [SMILES: CC1(C)Oc2cc3oc(=O)ccc3cc2CC1NC(=O)CBr ; CC(C)(C)OC(=O)NCCOCCOCCN ; CC(C)(C)OC(=O)NCCOCCOCCNCC(=O)NC1Cc2cc3ccc(=O)oc3cc2OC1(C)C]

A solution of tert-butyl (2-(2-(2-aminoethoxy)ethoxy)ethyl)carbamate (147 μL, 0.62 mmol) and triethylamine (TEA) (50 μL, 0.49 mmol) in acetonitrile was stirred at room temperature for 10 minutes. After 10 minutes, Compound 31 (150 mg, 0.41 mmol) was added, and the mixture was reacted at 40° C. for 7 hours. Upon completion of the reaction, the reaction mixture was quenched by addition of distilled water and extracted with ethyl acetate. The ethyl acetate extract was dried over anhydrous sodium sulfate and concentrated, and the concentrate was purified and separated by silica gel column chromatography using a mixture of dichloromethane and methanol (v/v, 50:1), thereby affording Compound 44a as a white solid (186 mg, 85%).

¹H NMR (400 MHz, CDCl₃): δ 7.65-7.54 (m, 2H), 7.19 (s, 1H), 6.83 (s, 1H), 6.26 (d, J=9.5 Hz, 1H), 5.01 (s, 1H), 4.30 (dt, J=9.3, 4.7 Hz, 1H), 3.54 (ddd, J=15.0, 9.9, 4.2 Hz, 5H), 3.49-3.28 (m, 6H), 3.28-3.08 (m, 2H), 2.82 (dd, J=17.1, 4.1 Hz, 1H), 2.78-2.59 (m, 2H), 1.45 (s, 9H), 1.39 (d, J=7.4 Hz, 6H).

MS (ESI): m/z [M+H]⁺ 534.3, found.

### 2) Preparation of 2-((2-(2-(2-aminoethoxy)ethoxy)ethyl)amino)-N-(8,8-dimethyl-2-oxo-7,8-dihydro-2H,6H-pyrano[3,2-g]chromen-7-yl)acetamide (Compound 44b)

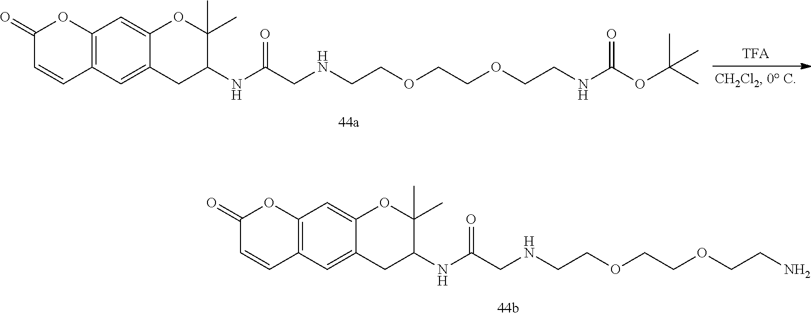 [SMILES: CC(C)(C)OC(=O)NCCOCCOCCNCC(=O)NC1Cc2cc3ccc(=O)oc3cc2OC1(C)C ; CC1(C)Oc2cc3oc(=O)ccc3cc2CC1NC(=O)CNCCOCCOCCN]

Compound 44a (150 mg, 0.28 mmol) was dissolved in dichloromethane, and the resulting solution was cooled at 0° C. for 10 minutes. After 10 minutes, trifluoroacetic acid (TFA) (500 μL) was slowly added, and the mixture was stirred for 1 hour. Upon completion of the reaction, the reaction mixture was concentrated. The concentrate was purified and separated by silica gel column chromatography using a mixture of dichloromethane and methanol (v/v, 15:1), thereby affording Compound 44b as a colorless semisolid (110 mg, 91%).

¹H NMR (400 MHz, CDCl₃): δ 7.50 (d, J=9.5 Hz, 1H), 7.10 (s, 2H), 6.67 (s, 1H), 6.14 (d, J=9.5 Hz, 1H), 4.20 (dt, J=9.0, 4.5 Hz, 1H), 3.69-3.63 (m, 2H), 3.58 (d, J=4.5 Hz, 2H), 3.53 (s, 4H), 3.48-3.44 (m, 2H), 3.42 (s, 1H), 3.08-3.02 (m, 3H), 2.90 (d, J=4.4 Hz, 2H), 2.72 (dd, J=17.2, 3.9 Hz, 1H), 1.28 (d, J=7.3 Hz, 6H).

MS (ESI): m/z [M+H]⁺ 434.2, found.

### 3) Preparation of N-(2-(2-(2-((2-((8,8-dimethyl-2-oxo-7,8-dihydro-2H,6H-pyrano[3,2-g]chromen-7-yl)amino)-2-oxoethyl)amino)ethoxy)ethoxy)ethyl)-3-((1,4-dioxo-1,4-dihydronaphthalen-2-yl)thio)propanamide (Compound 44)

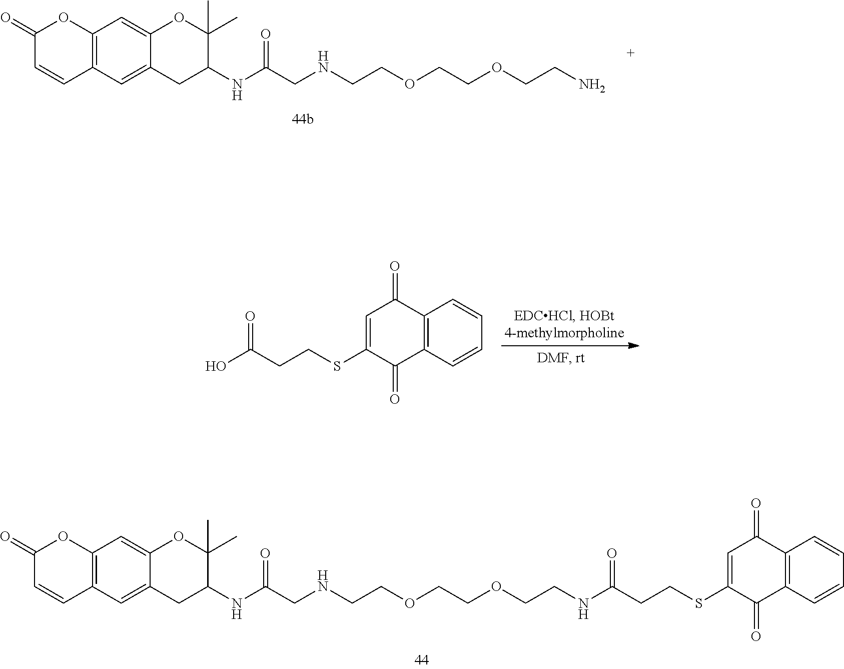 [SMILES: CC1(C)Oc2cc3oc(=O)ccc3cc2CC1NC(=O)CNCCOCCOCCN ; O=C(O)CCSC1=CC(=O)c2ccccc2C1=O ; CC1(C)Oc2cc3oc(=O)ccc3cc2CC1NC(=O)CNCCOCCOCCNC(=O)CCSC1=CC(=O)c2ccccc2C1=O]

Compound 44b (100 mg, 0.23 mmol), 3-((1,4-dioxo-1,4-dihydronaphthalen-2-yl)thio)propanoic acid (64 mg, 0.23 mmol), EDC·HCl (88 mg, 0.46 mmol), hydroxybenzotriazole (47 mg, 0.35 mmol), and 4-methylmorpholine (51 μL, 0.46 mmol) were dissolved in N,N-dimethylformamide (DMF), and the mixture was stirred at room temperature for 16 hours. Upon completion of the reaction, the reaction mixture was quenched by addition of 1 N aqueous hydrochloric acid and extracted with dichloromethane. The dichloromethane extract was washed several times with distilled water and brine, dried over anhydrous sodium sulfate, and concentrated. The concentrate was purified and separated by silica gel column chromatography using a mixture of dichloromethane and methanol (v/v, 50:1), thereby affording Compound 44 as a yellow solid (100 mg, 64%).

Mp: 128-130° C.

¹H NMR (400 MHz, CDCl₃): δ 7.50 (d, J=9.5 Hz, 1H), 7.10 (s, 2H), 6.67 (s, 1H), 6.14 (d, J=9.5 Hz, 1H), 4.20 (dt, J=9.0, 4.5 Hz, 1H), 3.69-3.63 (m, 2H), 3.58 (d, J=4.5 Hz, 2H), 3.53 (s, 4H), 3.48-3.44 (m, 2H), 3.42 (s, 1H), 3.08-3.02 (m, 3H), 2.90 (d, J=4.4 Hz, 2H), 2.72 (dd, J=17.2, 3.9 Hz, 1H), 1.28 (d, J=7.3 Hz, 6H).

¹³C NMR (101 MHz, CDCl₃): δ 182.13, 181.29, 170.69, 161.12, 156.37, 154.25, 153.78, 143.15, 134.60, 133.38, 131.97, 131.51, 129.34, 127.17, 126.71, 126.61, 115.95, 113.32, 112.98, 104.69, 77.46, 77.24, 70.47, 70.13, 48.87, 48.37, 48.36, 48.34, 47.77, 39.46, 33.41, 28.35, 26.41, 24.96, 24.11.

MS (ESI): m/z [M+H]⁺ 678.2, found.

### Example 44: Preparation of N-(8,8-dimethyl-2-oxo-7,8-dihydro-2H,6H-pyrano[3,2-g]chromen-7-yl)-4-((1,4-dioxo-1,4-dihydronaphthalen-2-yl)amino)butanamide (Compound 45)

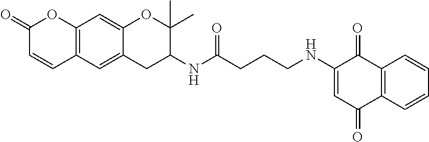 [SMILES: CC1(C)Oc2cc3oc(=O)ccc3cc2CC1NC(=O)CCCNC1=CC(=O)c2ccccc2C1=O]

Compound 1 (69 mg, 0.28 mmol) and 4-((1,4-dioxo-1,4-dihydronaphthalen-2-yl)amino)butanoic acid (80 mg, 0.34 mmol) were reacted according to the method described in Preparation Example 2. The dichloromethane extract was concentrated, and the residue was purified and separated by silica gel column chromatography using a mixture of dichloromethane and methanol (v/v, 50:1), thereby affording Compound 45 as a red solid (124 mg, 91%).

Mp: 212° C.

¹H NMR (400 MHz, CDCl₃): δ 8.00 (dd, J=16.4, 7.5 Hz, 2H), 7.70 (t, J=7.2 Hz, 1H), 7.58 (t, J=7.3 Hz, 1H), 7.45 (d, J=9.4 Hz, 1H), 7.09 (s, 1H), 6.60 (s, 1H), 6.38-6.24 (m, 2H), 6.10 (d, J=9.4 Hz, 1H), 5.65 (s, 1H), 4.42-4.33 (m, 1H), 3.27-3.14 (m, 3H), 2.76 (d, J=16.5 Hz, 1H), 2.36 (tt, J=16.1, 7.7 Hz, 2H), 2.11-2.01 (m, 2H), 1.41 (s, 3H), 1.33 (s, 3H).

¹³C NMR (101 MHz, CDCl₃): δ 182.89, 181.67, 172.09, 161.15, 156.44, 153.92, 148.12, 143.10, 134.69, 133.57, 131.99, 130.48, 129.35, 126.28, 126.08, 115.90, 113.32, 113.07, 104.94, 100.70, 77.61, 47.85, 42.25, 33.65, 28.81, 24.99, 24.85, 23.79.

MS (ESI): m/z [M+H]⁺ 487.2, found.

### Example 45: Preparation of N-(8,8-dimethyl-2-oxo-7,8-dihydro-2H,6H-pyrano[3,2-g]chromen-7-yl)-3-(naphthalen-2-ylthio)propanamide (Compound 46)

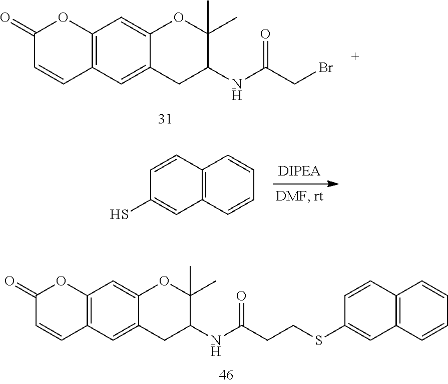 [SMILES: CC1(C)Oc2cc3oc(=O)ccc3cc2CC1NC(=O)CBr ; Sc1ccc2ccccc2c1 ; CC1(C)Oc2cc3oc(=O)ccc3cc2CC1NC(=O)CCSc1ccc2ccccc2c1]

A solution of 2-naphthalenethiol (43 mg, 0.27 mmol) and N,N-diisopropylethylamine (DIPEA) (63 μL, 0.36 mmol) in N,N-dimethylformamide (DMF) was stirred at room temperature for 10 minutes. After 10 minutes, Compound 31 (70 mg, 0.18 mmol) was added and the mixture was stirred for 16 hours. Upon completion of the reaction, the reaction mixture was quenched by addition of distilled water and extracted with ethyl acetate. The ethyl acetate extract was washed several times with distilled water and brine, dried over anhydrous sodium sulfate, and concentrated. The residue was purified and separated by silica gel column chromatography using a mixture of dichloromethane and methanol (v/v, 150:1), thereby affording Compound 46 as a white solid (60 mg, 73%).

Mp: 182° C.

¹H NMR (400 MHz, CDCl₃): δ 7.75 (d, J=7.8 Hz, 1H), 7.70-7.61 (m, 3H), 7.50 (d, J=9.4 Hz, 1H), 7.43 (p, J=6.7 Hz, 2H), 7.32 (d, J=8.5 Hz, 1H), 7.12 (s, 1H), 6.72 (s, 1H), 6.14 (d, J=9.4 Hz, 1H), 5.91 (d, J=8.9 Hz, 1H), 4.36-4.29 (m, 1H), 3.29 (t, J=6.9 Hz, 2H), 3.15 (dd, J=17.0, 4.9 Hz, 1H), 2.81-2.72 (m, 1H), 2.49 (tt, J=15.1, 7.3 Hz, 2H), 1.41 (s, 3H), 1.33 (s, 3H).

¹³C NMR (101 MHz, CDCl₃): δ 170.82, 161.05, 156.34, 154.08, 143.00, 133.66, 132.48, 131.93, 129.31, 128.62, 127.86, 127.72, 127.59, 127.03, 126.71, 125.99, 115.90, 113.53, 113.22, 105.06, 77.54, 48.04, 35.99, 29.55, 28.77, 24.84.

MS (ESI): m/z [M+H]⁺ 460.2, found.

### Example 46: Preparation of N-(8,8-dimethyl-2-oxo-7,8-dihydro-2H,6H-pyrano[3,2-g]chromen-7-yl)-3-((1,4-dioxo-1,4-dihydronaphthalen-2-yl)amino)propanamide (Compound 47)

### 1) Preparation of 3-bromo-N-(8,8-dimethyl-2-oxo-7,8-dihydro-2H,6H-pyrano[3,2-g]chromen-7-yl)propanamide (Compound 47a)

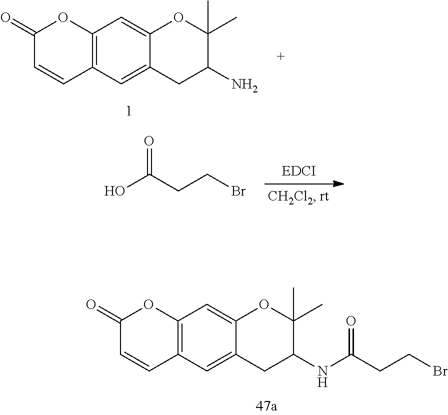 [SMILES: CC1(C)Oc2cc3oc(=O)ccc3cc2CC1N ; O=C(O)CCBr ; CC1(C)Oc2cc3oc(=O)ccc3cc2CC1NC(=O)CCBr]

A solution of Compound 1 (100 mg, 0.40 mmol), 3-bromopropionic acid (92 mg, 0.60 mmol), and EDC·HCl (153 mg, 0.80 mmol) in dichloromethane was stirred at room temperature for 4 hours. Upon completion of the reaction, distilled water was added to quench the reaction, and the mixture was extracted with dichloromethane. The dichloromethane extract was dried over anhydrous sodium sulfate and concentrated, and the residue was purified and separated by silica gel column chromatography using a mixture of dichloromethane and methanol (v/v, 100:1), thereby affording Compound 47a as an ivory solid (114 mg, 75%).

¹H NMR (400 MHz, CDCl₃): δ 7.58 (d, J=9.5 Hz, 1H), 7.19 (s, 1H), 6.77 (s, 1H), 6.23 (d, J=9.5 Hz, 1H), 5.86 (d, J=8.9 Hz, 1H), 4.36 (ddd, J=8.6, 5.1, 3.1 Hz, 1H), 3.62 (dtt, J=10.1, 6.2, 3.2 Hz, 2H), 3.19 (dd, J=17.0, 5.2 Hz, 1H), 2.80 (dd, J=17.1, 2.9 Hz, 1H), 2.74 (t, J=6.4 Hz, 2H), 1.44 (s, 3H), 1.35 (s, 3H).

MS (ESI): m/z [M+H]⁺ 380.1, found.

### 2) Preparation of 3-azido-N-(8,8-dimethyl-2-oxo-7,8-dihydro-2H,6H-pyrano[3,2-g]chromen-7-yl)propanamide (Compound 47b)

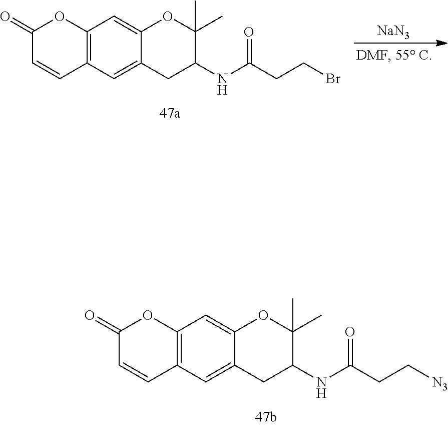 [SMILES: CC1(C)Oc2cc3oc(=O)ccc3cc2CC1NC(=O)CCBr ; CC1(C)Oc2cc3oc(=O)ccc3cc2CC1NC(=O)CCN=[N+]=[N-] ; "NaN3"]

A solution of Compound 47a (110 mg, 0.29 mmol) and sodium azide (34 mg, 0.52 mmol) in N,N-dimethylformamide (DMF) was stirred at 55° C. for 1 hour. Upon completion of the reaction, distilled water was added to quench the reaction, and the mixture was extracted with ethyl acetate. The ethyl acetate extract was washed several times with water and brine, dried over anhydrous sodium sulfate, and concentrated. The residue was purified and separated by silica gel column chromatography using a mixture of dichloromethane and methanol (v/v, 100:1), thereby affording Compound 47b as a white solid (94 mg, 95%).

¹H NMR (400 MHz, CDCl₃): δ 7.58 (d, J=9.4 Hz, 1H), 7.19 (s, 1H), 6.77 (s, 1H), 6.23 (d, J=9.4 Hz, 1H), 5.86 (d, J=8.9 Hz, 1H), 4.36 (dq, J=8.4, 3.0 Hz, 1H), 3.61 (t, J=6.3 Hz, 2H), 3.19 (dd, J=16.9, 4.9 Hz, 1H), 2.79 (dd, J=17.0, 2.8 Hz, 1H), 2.39 (qd, J=9.0, 7.6, 5.5 Hz, 2H), 1.43 (s, 3H), 1.35 (s, 3H).

MS (ESI): m/z [M+H]⁺ 343.1, found.

### 3) Preparation of 3-amino-N-(8,8-dimethyl-2-oxo-7,8-dihydro-2H,6H-pyrano[3,2-g]chromen-7-yl)propanamide (Compound 47c)

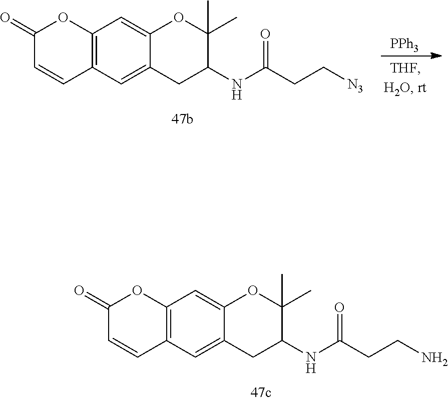 [SMILES: CC1(C)Oc2cc3oc(=O)ccc3cc2CC1NC(=O)CCN=[N+]=[N-] ; CC1(C)Oc2cc3oc(=O)ccc3cc2CC1NC(=O)CCN ; "PPh3"]

A solution of Compound 47b (100 mg, 0.29 mmol), triphenylphosphine (152 mg, 0.58 mmol), and water (13 μL) in tetrahydrofuran (THF) was stirred at room temperature for 18 hours. Upon completion of the reaction, saturated sodium bicarbonate solution was added to quench the reaction, and the mixture was extracted with dichloromethane. The dichloromethane extract was dried over anhydrous sodium sulfate and concentrated, and the residue was purified and separated by silica gel column chromatography using a mixture of dichloromethane and methanol (v/v, 20:1), thereby affording Compound 47c as a white solid (34 mg, 37%).

¹H NMR (400 MHz, CDCl₃): δ 7.77 (d, J=8.4 Hz, 1H), 7.58 (d, J=9.5 Hz, 1H), 7.17 (s, 1H), 6.79 (s, 1H), 6.24 (d, J=9.5 Hz, 1H), 4.31 (dt, J=8.9, 4.5 Hz, 1H), 3.16 (dd, J=16.8, 4.9 Hz, 1H), 3.02-2.88 (m, 2H), 2.78 (dd, J=16.9, 3.7 Hz, 1H), 2.29 (dd, J=6.8, 4.6 Hz, 2H), 1.40 (s, 3H), 1.35 (s, 3H).

MS (ESI): m/z [M+H]⁺ 317.1, found.

### 4) Preparation of N-(8,8-dimethyl-2-oxo-7,8-dihydro-2H,6H-pyrano[3,2-g]chromen-7-yl)-3-((1,4-dioxo-1,4-dihydronaphthalen-2-yl)amino)propanamide (Compound 47)

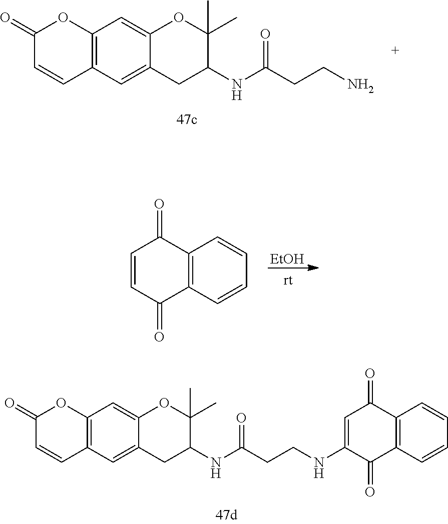 [SMILES: "EtOH" ; CC1(C)Oc2cc3oc(=O)ccc3cc2CC1NC(=O)CCN ; O=C1C=CC(=O)c2ccccc21 ; CC1(C)Oc2cc3oc(=O)ccc3cc2CC1NC(=O)CCNC1=CC(=O)c2ccccc2C1=O]

A solution of Compound 47c (30 mg, 0.09 mmol) and 1,4-naphthoquinone (16 mg, 0.10 mmol) in ethanol was stirred at room temperature for 1 hour. Upon completion of the reaction, the precipitate was collected by vacuum filtration, washed with distilled water, and dried to afford Compound 47 as an orange solid (23 mg, 55%).

Mp: 204° C.

¹H NMR (400 MHz, CDCl₃): δ 7.77 (d, J=8.4 Hz, 1H), 7.58 (d, J=9.5 Hz, 1H), 7.17 (s, 1H), 6.79 (s, 1H), 6.24 (d, J=9.5 Hz, 1H), 4.31 (dt, J=8.9, 4.5 Hz, 1H), 3.16 (dd, J=16.8, 4.9 Hz, 1H), 3.02-2.88 (m, 2H), 2.78 (dd, J=16.9, 3.7 Hz, 1H), 2.29 (dd, J=6.8, 4.6 Hz, 2H), 1.40 (s, 3H), 1.35 (s, 3H).

¹³C NMR (101 MHz, CDCl₃): δ 182.91, 181.54, 170.36, 161.20, 156.42, 153.90, 147.73, 143.15, 134.72, 133.48, 132.04, 130.48, 129.37, 126.27, 126.14, 115.81, 113.36, 113.10, 104.94, 100.88, 77.51, 47.96, 38.66, 34.20, 28.74, 25.00, 24.83.

MS (ESI): m/z [M+H]⁺ 473.2, found.

### Example 47: Preparation of (2R)-2-acetamido-N-(8,8-dimethyl-2-oxo-7,8-dihydro-2H,6H-pyrano[3,2-g]chromen-7-yl)-3-mercaptopropanamide (Compound 48)

### 1) Preparation of (2R)-2-acetamido-N-(8,8-dimethyl-2-oxo-7,8-dihydro-2H,6H-pyrano[3,2-g]chromen-7-yl)-3-(tritylthio)propanamide (Compound 48a)

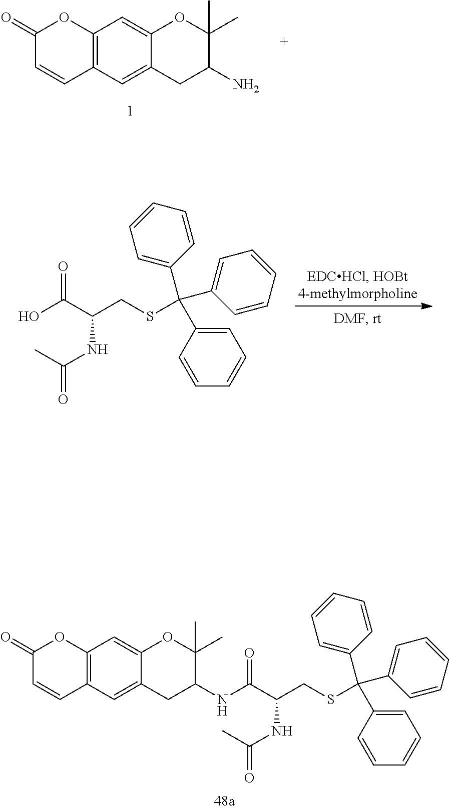 [SMILES: CC1(C)Oc2cc3oc(=O)ccc3cc2CC1N ; CC(=O)N[C@@H](CSC(c1ccccc1)(c1ccccc1)c1ccccc1)C(=O)O ; CC(=O)N[C@@H](CSC(c1ccccc1)(c1ccccc1)c1ccccc1)C(=O)NC1Cc2cc3ccc(=O)oc3cc2OC1(C)C]

A solution of Compound 1 (250 mg, 1.02 mmol), N-acetyl-S-trityl-L-cysteine (495 mg, 1.22 mmol), EDC·HCl (495 mg, 2.04 mmol), hydroxybenzotriazole (207 mg, 1.53 mmol), and 4-methylmorpholine (224 μL, 2.04 mmol) in N,N-dimethylformamide (DMF) was stirred at room temperature for 1 hour. Upon completion of the reaction, the reaction mixture was quenched with 1 N aqueous hydrochloric acid and extracted with ethyl acetate. The ethyl acetate extract was washed several times with water and brine, dried over anhydrous sodium sulfate, and concentrated, and the residue was purified by silica gel column chromatography using a mixture of dichloromethane and methanol (v/v, 100:1), thereby affording Compound 48a as a white solid (536 mg, 83%).

¹H NMR (400 MHz, CDCl₃): δ 7.40 (dd, J=14.0, 8.6 Hz, 2H), 7.32-7.16 (m, 16H), 7.01 (s, 1H), 6.80 (s, 1H), 6.20 (d, J=9.5 Hz, 1H), 6.09 (dd, J=8.9, 3.7 Hz, 1H), 5.85 (dd, J=7.0, 3.8 Hz, 1H), 4.15 (dh, J=9.2, 5.0 Hz, 1H), 3.52-3.44 (m, 1H), 3.07 (dd, J=17.0, 4.8 Hz, 1H), 2.79 (dd, J=17.0, 3.5 Hz, 1H), 2.68 (dd, J=13.5, 6.3 Hz, 1H), 2.51 (dd, J=13.4, 7.5 Hz, 1H), 1.82 (s, 3H), 1.31 (s, 4H), 1.29 (s, 3H).

MS (ESI): m/z [M+H]⁺ 633.2, found.

### 2) Preparation of (2R)-2-acetamido-N-(8,8-dimethyl-2-oxo-7,8-dihydro-2H,6H-pyrano[3,2-g]chromen-7-yl)-3-mercaptopropanamide (Compound 48)

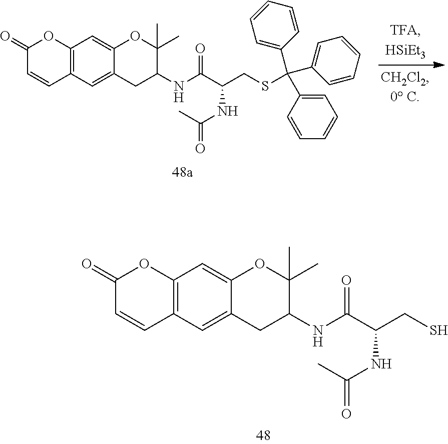 [SMILES: CC(=O)N[C@@H](CSC(c1ccccc1)(c1ccccc1)c1ccccc1)C(=O)NC1Cc2cc3ccc(=O)oc3cc2OC1(C)C ; CC(=O)N[C@@H](CS)C(=O)NC1Cc2cc3ccc(=O)oc3cc2OC1(C)C]

A solution of Compound 48a (500 mg, 0.79 mmol) in dichloromethane was cooled at 0° C. for 10 minutes. After 10 minutes, trifluoroacetic acid (TFA; 3000 μL) and triethylsilane (300 μL) were simultaneously added slowly, and the mixture was stirred for 1.5 hours. Upon completion of the reaction, the reaction mixture was concentrated under reduced pressure, and the residue was purified by silica gel column chromatography using a mixture of dichloromethane and methanol (v/v, 100:1), thereby affording Compound 48 as a white semisolid (305 mg, 99%).

Mp: 179° C.

¹H NMR (400 MHz, CDCl₃): δ 7.56 (dd, J=15.1, 9.5 Hz, 1H), 7.16 (d, J=12.6 Hz, 1H), 6.96 (d, J=9.0 Hz, 1H), 6.72 (d, J=10.4 Hz, 1H), 6.61 (d, J=7.4 Hz, 1H), 6.20 (dd, J=13.4, 9.5 Hz, 1H), 4.65-4.56 (m, 1H), 4.29 (dt, J=8.8, 4.4 Hz, 1H), 3.16 (dt, J=17.0, 4.8 Hz, 1H), 2.95 (dddt, J=13.5, 8.0, 5.9, 2.9 Hz, 1H), 2.78 (ddd, J=17.1, 8.5, 4.4 Hz, 2H), 2.17 (s, 1H), 1.59-1.52 (m, 1H), 1.40 (d, J=21.2 Hz, 3H), 1.35 (d, J=8.1 Hz, 3H).

¹³C NMR (101 MHz, CDCl₃): δ 170.16, 169.79, 161.18, 156.38, 154.08, 143.15, 143.10, 129.09, 115.91, 113.51, 113.14, 105.06, 54.42, 48.64, 28.70, 26.88, 25.11, 24.26, 23.09.

MS (ESI): m/z [M+H]⁺ 391.1, found.

### Example 48: Preparation of S-((2R)-2-acetamido-3-((8,8-dimethyl-2-oxo-7,8-dihydro-2H,6H-pyrano[3,2-g]chromen-7-yl)amino)-3-oxopropyl) ethanethioate (Compound 49)

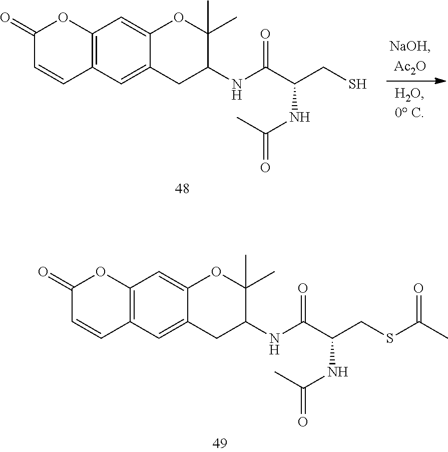 [SMILES: CC(=O)N[C@@H](CS)C(=O)NC1Cc2cc3ccc(=O)oc3cc2OC1(C)C ; CC(=O)N[C@@H](CSC(C)=O)C(=O)NC1Cc2cc3ccc(=O)oc3cc2OC1(C)C]

A solution of Compound 48 (50 mg, 0.13 mmol) and sodium hydroxide (16 mg, 0.39 mmol) in distilled water was stirred at 0° C. for 10 minutes. After 10 minutes, acetic anhydride (25 μL, 0.26 mmol) was slowly added, and the mixture was stirred for 2 hours. Upon completion of the reaction, the reaction mixture was neutralized with 3 N aqueous hydrochloric acid and extracted with ethyl acetate. The ethyl acetate extract was dried over anhydrous sodium sulfate and concentrated, and the residue was purified and separated by silica gel column chromatography using a mixture of dichloromethane and methanol (v/v, 100:1), thereby affording Compound 49 as a white solid (46 mg, 82%).

Mp: 258° C.

¹H NMR (400 MHz, DMSO): δ 8.18 (dd, J=17.3, 8.5 Hz, 2H), 7.93 (d, J=9.5 Hz, 1H), 7.48 (s, 1H), 6.79 (s, 1H), 6.27 (d, J=9.5 Hz, 1H), 4.49 (q, J=7.1 Hz, 1H), 4.11 (q, J=8.2 Hz, 1H), 3.15 (dd, J=13.2, 6.5 Hz, 1H), 3.06 (dd, J=13.2, 7.3 Hz, 1H), 2.99 (dd, J=17.0, 5.5 Hz, 1H), 2.73 (dd, J=16.9, 8.0 Hz, 1H), 2.33 (s, 3H), 1.82 (s, 3H), 1.32 (s, 3H), 1.25 (s, 3H).

¹³C NMR (101 MHz, DMSO): δ 194.90, 170.26, 169.63, 160.75, 156.55, 154.02, 144.60, 129.86, 117.83, 113.16, 112.98, 104.11, 78.60, 52.05, 48.59, 31.35, 30.98, 28.20, 26.16, 22.84, 22.13.

MS (ESI): m/z [M+H]⁺ 433.1, found.

### Preparation Example 3: Preparation of Decursin Urea Derivatives (VIII)

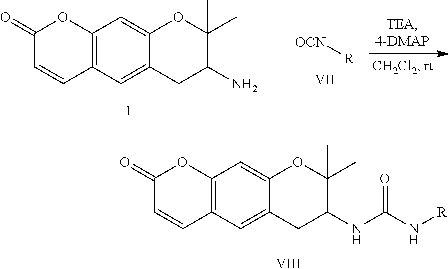

Urea derivatives of Formula (VI) were prepared by reacting Compound 1, prepared in Preparation Example 1, with isocyanates of Formula (VII). A general reaction procedure for the urea derivatives is as follows.

Compound 1 (1.0 equiv), isocyanate VII (1.2 equiv), triethylamine (TEA, 1.5 equiv), and 4-dimethylaminopyridine (4-DMAP, 0.6 equiv) were dissolved in dichloromethane, and the mixture was stirred at room temperature for 14 hours. Upon completion of the reaction, a saturated aqueous ammonium chloride solution was added to quench the reaction, and the mixture was extracted with dichloromethane. The dichloromethane extract was dried over anhydrous sodium sulfate and concentrated, and the concentrate was purified by silica gel column chromatography using a mixture of dichloromethane and methanol, thereby affording the decursin urea derivatives in solid form.

The preparation of specific decursin urea derivatives is described in the following Examples.

### Example 49: Preparation of 1-(8,8-dimethyl-2-oxo-7,8-dihydro-2H,6H-pyrano[3,2-g]chromen-7-yl)-3-phenylurea (Compound 50)

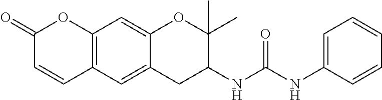 [SMILES: CC1(C)Oc2cc3oc(=O)ccc3cc2CC1NC(=O)Nc1ccccc1]

Compound 1 (108 mg, 0.44 mmol) and phenyl isocyanate (58 μL, 0.53 mmol) were reacted according to the method described in Preparation Example 3. The dichloromethane extract concentrate was purified and separated by silica gel column chromatography using a mixture of dichloromethane and methanol (v/v, 50:1), thereby affording Compound 50 as a white solid (121 mg, 75%).

Mp: 295° C.

¹H NMR (400 MHz, DMSO): δ 8.40 (s, 1H), 7.94 (d, J=9.5 Hz, 1H), 7.50 (s, 1H), 7.35 (d, J=7.8 Hz, 2H), 7.21 (t, J=7.9 Hz, 2H), 6.88 (dd, J=14.7, 7.4 Hz, 2H), 6.29 (dd, J=12.9, 9.2 Hz, 2H), 4.08-4.01 (m, 1H), 3.14 (dd, J=16.8, 4.8 Hz, 1H), 2.74 (dd, J=17.0, 4.7 Hz, 1H), 1.34 (s, 6H).

¹³C NMR (101 MHz, DMSO): δ 160.73, 156.65, 155.18, 154.05, 144.59, 140.70, 130.39, 129.18, 121.63, 117.88, 117.45, 113.20, 113.15, 104.13, 78.75, 48.26, 29.12, 25.60, 23.99.

MS (ESI): m/z [M+H]⁺ 365.2, found.

### Example 50: Preparation of 1-benzyl-3-(8,8-dimethyl-2-oxo-7,8-dihydro-2H,6H-pyrano[3,2-g]chromen-7-yl)urea (Compound 51)

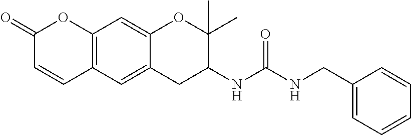 [SMILES: CC1(C)Oc2cc3oc(=O)ccc3cc2CC1NC(=O)NCc1ccccc1]

Compound 1 (110 mg, 0.45 mmol) and benzyl isocyanate (67 μL, 0.55 mmol) were reacted according to the method described in Preparation Example 3. The dichloromethane extract concentrate was purified and separated by silica gel column chromatography using a mixture of dichloromethane and methanol (v/v, 30:1), thereby affording Compound 51 as a white solid (159 mg, 93%).

Mp: 193-195° C.

¹H NMR (400 MHz, DMSO): δ 7.93 (d, J=9.5 Hz, 1H), 7.48 (s, 1H), 7.35-7.26 (m, 2H), 7.25-7.17 (m, 3H), 6.80 (s, 1H), 6.31 (t, J=5.9 Hz, 1H), 6.26 (d, J=9.5 Hz, 1H), 6.08 (d, J=9.1 Hz, 1H), 4.28-4.15 (m, 2H), 3.99 (dt, J=9.2, 5.5 Hz, 1H), 3.07 (dd, J=17.0, 5.1 Hz, 1H), 2.67 (dd, J=16.9, 5.8 Hz, 1H), 1.29 (d, J=10.5 Hz, 6H).

¹³C NMR (101 MHz, DMSO): δ 160.75, 158.06, 156.73, 154.01, 144.60, 141.11, 130.21, 128.73, 127.40, 127.08, 117.82, 113.10, 113.02, 104.04, 79.01, 48.58, 43.35, 29.29, 25.82, 23.46, 22.20, 14.36.

MS (ESI): m/z [M+H]⁺ 379.2, found.

### Example 51: Preparation of 1-(8,8-dimethyl-2-oxo-7,8-dihydro-2H,6H-pyrano[3,2-g]chromen-7-yl)-3-phenethylurea (Compound 52)

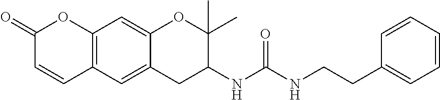 [SMILES: CC1(C)Oc2cc3oc(=O)ccc3cc2CC1NC(=O)NCCc1ccccc1]

Compound 1 (121 mg, 0.49 mmol) and phenethyl isocyanate (82 μL, 0.59 mmol) were reacted according to the method described in Preparation Example 3. The dichloromethane extract concentrate was purified and separated by silica gel column chromatography using a mixture of dichloromethane and methanol (v/v, 30:1), thereby affording Compound 52 as a white solid (174 mg, 90%).

Mp: 109° C.

¹H NMR (400 MHz, DMSO): δ 7.93 (d, J=9.5 Hz, 1H), 7.46 (s, 1H), 7.32-7.22 (m, 2H), 7.19 (dd, J=7.2, 5.0 Hz, 3H), 6.79 (s, 1H), 6.26 (d, J=9.5 Hz, 1H), 6.02 (d, J=9.0 Hz, 1H), 5.85 (t, J=5.7 Hz, 1H), 3.95 (dt, J=9.1, 5.5 Hz, 1H), 3.30-3.19 (m, 2H), 3.04 (dd, J=17.0, 5.1 Hz, 1H), 2.70-2.59 (m, 3H), 1.29 (s, 3H), 1.25 (s, 3H).

¹³C NMR (101 MHz, DMSO): δ 160.74, 156.65, 155.18, 154.04, 144.59, 140.69, 130.39, 129.19, 128.81, 121.63, 117.87, 117.45, 113.20, 113.15, 104.13, 78.75, 48.26, 29.12, 25.59, 23.99.

MS (ESI): m/z [M+H]⁺ 393.2, found.

### Example 52: Preparation of 1-(8,8-dimethyl-2-oxo-7,8-dihydro-2H,6H-pyrano[3,2-g]chromen-7-yl)-3-(3-phenylpropyl)urea (Compound 53)

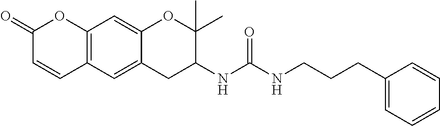 [SMILES: CC1(C)Oc2cc3oc(=O)ccc3cc2CC1NC(=O)NCCCc1ccccc1]

Compound 1 (98 mg, 0.40 mmol) and 3-phenylpropyl isocyanate (74 μL, 0.48 mmol) were reacted according to the method described in Preparation Example 3. The dichloromethane extract concentrate was purified and separated by silica gel column chromatography using a mixture of dichloromethane and methanol (v/v, 30:1), thereby affording Compound 53 as a white solid (131 mg, 81%).

Mp: 142° C.

¹H NMR (400 MHz, DMSO): δ 7.92 (d, J=9.5 Hz, 1H), 7.47 (s, 1H), 7.27 (t, J=7.4 Hz, 2H), 7.17 (d, J=7.2 Hz, 3H), 6.80 (s, 1H), 6.26 (d, J=9.5 Hz, 1H), 5.94 (d, J=8.9 Hz, 2H), 3.96 (dt, J=9.3, 5.3 Hz, 1H), 3.11-2.95 (m, 3H), 2.66 (dd, J=16.8, 5.5 Hz, 1H), 2.57-2.52 (m, 2H), 1.70-1.57 (m, 2H), 1.28 (d, J=9.6 Hz, 6H).

¹³C NMR (101 MHz, DMSO): δ 160.75, 158.07, 156.74, 154.00, 144.59, 142.20, 130.21, 128.74, 128.71, 126.17, 117.86, 113.09, 113.01, 104.04, 79.04, 48.46, 32.91, 32.20, 29.30, 25.82, 23.47.

MS (ESI): m/z [M+H]⁺ 407.2, found.

### Example 53: Preparation of 1-(8,8-dimethyl-2-oxo-7,8-dihydro-2H,6H-pyrano[3,2-g]chromen-7-yl)-3-(4-phenylbutyl)urea (Compound 54)

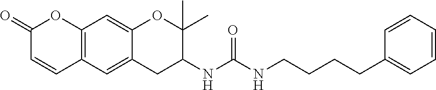 [SMILES: CC1(C)Oc2cc3oc(=O)ccc3cc2CC1NC(=O)NCCCCc1ccccc1]

Compound 1 (96 mg, 0.39 mmol) and 4-phenylbutyl isocyanate (81 μL, 0.47 mmol) were reacted according to the method described in Preparation Example 3. The dichloromethane extract concentrate was purified and separated by silica gel column chromatography using a mixture of dichloromethane and methanol (v/v, 50:1), thereby affording Compound 54 as a white solid (115 mg, 70%).

Mp: 122° C.

¹H NMR (400 MHz, DMSO): δ 7.92 (d, J=9.5 Hz, 1H), 7.46 (s, 1H), 7.28-7.23 (m, 2H), 7.20-7.13 (m, 3H), 6.79 (s, 1H), 6.26 (d, J=9.5 Hz, 1H), 5.89 (d, J=9.1 Hz, 1H), 5.84 (t, J=5.6 Hz, 1H), 4.00-3.87 (m, 1H), 3.08-2.96 (m, 3H), 2.64 (dd, J=16.9, 5.7 Hz, 1H), 2.55 (t, J=7.6 Hz, 2H), 1.58-1.47 (m, 2H), 1.41-1.31 (m, 2H), 1.28 (s, 3H), 1.25 (s, 3H).

¹³C NMR (101 MHz, DMSO): δ 160.75, 158.05, 156.74, 154.00, 144.60, 142.64, 130.20, 128.72, 128.68, 126.10, 117.86, 114.16, 113.08, 113.00, 104.02, 79.03, 48.43, 35.27, 30.05, 29.30, 28.82, 25.82, 23.45.

MS (ESI): m/z [M+H]⁺ 421.2, found.

### Example 54: Preparation of 1-(4-chlorophenyl)-3-(8,8-dimethyl-2-oxo-7,8-dihydro-2H,6H-pyrano[3,2-g]chromen-7-yl)urea (Compound 55)

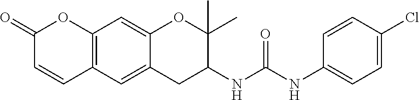 [SMILES: CC1(C)Oc2cc3oc(=O)ccc3cc2CC1NC(=O)Nc1ccc(Cl)cc1]

Compound 1 (130 mg, 0.53 mmol) and 4-chlorophenyl isocyanate (82 μL, 0.64 mmol) were reacted according to the method described in Preparation Example 3. The dichloromethane extract concentrate was purified and separated by silica gel column chromatography using a mixture of dichloromethane and methanol (v/v, 50:1), thereby affording Compound 55 as a white solid (123 mg, 58%).

Mp: 158° C.

¹H NMR (400 MHz, DMSO): δ 8.54 (s, 1H), 7.94 (d, J=9.5 Hz, 1H), 7.50 (s, 1H), 7.38 (d, J=8.9 Hz, 2H), 7.25 (d, J=8.9 Hz, 2H), 6.85 (s, 1H), 6.35 (d, J=8.9 Hz, 1H), 6.28 (d, J=9.5 Hz, 1H), 4.08-4.00 (m, 1H), 3.14 (dd, J=17.0, 5.0 Hz, 1H), 2.74 (dd, J=17.0, 4.8 Hz, 1H), 1.33 (s, 6H).

¹³C NMR (101 MHz, DMSO): δ 160.73, 156.61, 155.04, 154.05, 144.58, 139.66, 130.39, 129.01, 125.10, 119.40, 117.40, 113.22, 113.17, 104.14, 78.70, 48.34, 29.05, 25.56, 23.99.

MS (ESI): m/z [M+H]⁺ 399.1, found.

### Example 55: Preparation of 1-(8,8-dimethyl-2-oxo-7,8-dihydro-2H,6H-pyrano[3,2-g]chromen-7-yl)-3-(4-methoxyphenyl)urea (Compound 56)

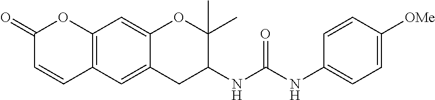 [SMILES: COc1ccc(NC(=O)NC2Cc3cc4ccc(=O)oc4cc3OC2(C)C)cc1]

Compound 1 (130 mg, 0.53 mmol) and 4-methoxyphenyl isocyanate (83 μL, 0.64 mmol) were reacted according to the method described in Preparation Example 3. The dichloromethane extract concentrate was purified and separated by silica gel column chromatography using a mixture of dichloromethane and methanol (v/v, 30:1), thereby affording Compound 56 as a white solid (179 mg, 86%).

Mp: 169° C.

¹H NMR (400 MHz, DMSO): δ 8.21 (s, 1H), 7.94 (d, J=9.5 Hz, 1H), 7.50 (s, 1H), 7.25 (d, J=9.0 Hz, 2H), 6.84 (s, 1H), 6.80 (d, J=9.0 Hz, 2H), 6.27 (d, J=9.5 Hz, 1H), 6.19 (d, J=8.9 Hz, 1H), 4.07-3.99 (m, 1H), 3.68 (s, 3H), 3.13 (dd, J=17.0, 5.0 Hz, 1H), 2.73 (dd, J=17.0, 4.9 Hz, 1H), 1.33 (s, 6H).

¹³C NMR (101 MHz, DMSO): δ 160.73, 156.67, 155.38, 154.44, 154.04, 144.59, 133.87, 130.37, 119.54, 117.53, 114.42, 113.18, 113.13, 104.11, 78.81, 55.61, 48.30, 29.17, 25.65, 23.91.

MS (ESI): m/z [M+H]⁺ 395.1, found.

### Example 56: Preparation of 1-cyclopentyl-3-(8,8-dimethyl-2-oxo-7,8-dihydro-2H,6H-pyrano[3,2-g]chromen-7-yl)urea (Compound 57)

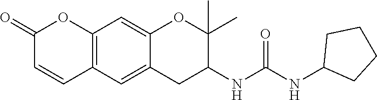 [SMILES: CC1(C)Oc2cc3oc(=O)ccc3cc2CC1NC(=O)NC1CCCC1]

Compound 1 (130 mg, 0.53 mmol) and cyclopentyl isocyanate (72 μL, 0.64 mmol) were reacted according to the method described in Preparation Example 3. The dichloromethane extract concentrate was purified and separated by silica gel column chromatography using a mixture of dichloromethane and methanol (v/v, 30:1), thereby affording Compound 57 as an ivory solid (148 mg, 78%).

Mp: 241° C.

¹H NMR (400 MHz, DMSO): δ 7.93 (d, J=9.5 Hz, 1H), 7.47 (s, 1H), 6.79 (s, 1H), 6.26 (d, J=9.5 Hz, 1H), 5.87 (d, J=7.3 Hz, 1H), 5.78 (d, J=9.1 Hz, 1H), 3.98-3.89 (m, 1H), 3.84 (dd, J=13.3, 6.6 Hz, 1H), 3.05 (dd, J=17.0, 5.1 Hz, 1H), 2.63 (dd, J=16.9, 5.5 Hz, 1H), 1.75 (dt, J=10.7, 5.3 Hz, 2H), 1.58-1.44 (m, 4H), 1.28 (s, 3H), 1.27-1.20 (m, 5H).

¹³C NMR (101 MHz, DMSO): δ 160.75, 157.64, 156.75, 154.00, 144.60, 130.24, 117.81, 113.08, 113.00, 104.01, 79.03, 51.35, 48.31, 33.52, 33.41, 29.32, 25.79, 23.57, 23.56, 23.54.

MS (ESI): m/z [M+H]⁺ 357.2, found.

### Example 57: Preparation of 1-cyclohexyl-3-(8,8-dimethyl-2-oxo-7,8-dihydro-2H,6H-pyrano[3,2-g]chromen-7-yl)urea (Compound 58)

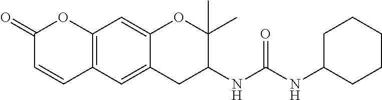 [SMILES: CC1(C)Oc2cc3oc(=O)ccc3cc2CC1NC(=O)NC1CCCCC1]

Compound 1 (95 mg, 0.39 mmol) and cyclohexyl isocyanate (60 μL, 0.47 mmol) were reacted according to the method described in Preparation Example 3. The dichloromethane extract concentrate was purified and separated by silica gel column chromatography using a mixture of dichloromethane and methanol (v/v, 50:1), thereby affording Compound 58 as a white solid (95 mg, 66%).

Mp: 238° C.

¹H NMR (400 MHz, DMSO): δ 7.93 (d, J=9.5 Hz, 1H), 7.47 (s, 1H), 6.79 (s, 1H), 6.26 (d, J=9.5 Hz, 1H), 5.82 (dd, J=12.0, 8.7 Hz, 2H), 3.94 (dt, J=9.4, 5.3 Hz, 1H), 3.37 (dd, J=10.7, 6.8 Hz, 1H), 3.05 (dd, J=16.8, 4.9 Hz, 1H), 2.63 (dd, J=16.8, 5.4 Hz, 1H), 1.72 (s, 2H), 1.64-1.55 (m, 2H), 1.49 (dd, J=8.2, 3.6 Hz, 1H), 1.28 (s, 3H), 1.26 (s, 3H), 1.21 (s, 2H), 1.19-1.11 (m, 1H), 1.06 (dd, J=10.2, 7.3 Hz, 2H).

¹³C NMR (101 MHz, DMSO): δ 160.75, 157.33, 156.76, 154.00, 144.60, 130.23, 117.82, 113.07, 112.99, 104.01, 79.07, 48.27, 48.00, 33.65, 33.57, 29.31, 25.79, 25.74, 24.73, 24.70, 23.56.

MS (ESI): m/z [M+H]⁺ 371.2, found.

### Example 58: Preparation of 1-(8,8-dimethyl-2-oxo-7,8-dihydro-2H,6H-pyrano[3,2-g]chromen-7-yl)-3-propylurea (Compound 59)

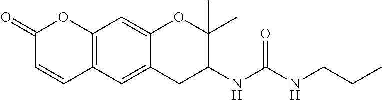 [SMILES: CCCNC(=O)NC1Cc2cc3ccc(=O)oc3cc2OC1(C)C]

Compound 1 (150 mg, 0.61 mmol) and propyl isocyanate (68 μL, 0.73 mmol) were reacted according to the method described in Preparation Example 3. The dichloromethane extract concentrate was purified and separated by silica gel column chromatography using a mixture of dichloromethane and methanol (v/v, 50:1), thereby affording Compound 59 as a white solid (147 mg, 73%).

Mp: 180° C.

¹H NMR (400 MHz, DMSO): δ 7.93 (d, J=9.5 Hz, 1H), 7.47 (s, 1H), 6.79 (s, 1H), 6.26 (d, J=9.5 Hz, 1H), 5.91 (d, J=9.1 Hz, 1H), 5.85 (t, J=5.6 Hz, 1H), 3.94 (dt, J=9.2, 5.5 Hz, 1H), 3.04 (dd, J=16.9, 5.1 Hz, 1H), 2.99-2.89 (m, 2H), 2.64 (dd, J=16.9, 5.7 Hz, 1H), 1.35 (dt, J=14.4, 7.2 Hz, 2H), 1.29 (s, 3H), 1.26 (s, 3H), 0.81 (t, J=7.4 Hz, 3H).

¹³C NMR (101 MHz, DMSO): δ 160.25, 157.56, 156.25, 153.51, 144.11, 129.71, 117.38, 112.59, 112.50, 103.53, 78.55, 47.93, 40.93, 28.81, 25.33, 23.11, 22.95, 11.27.

MS (ESI): m/z [M+H]⁺ 331.1, found.

### Example 59: Preparation of 1-(8,8-dimethyl-2-oxo-7,8-dihydro-2H,6H-pyrano[3,2-g]chromen-7-yl)-3-hexylurea (Compound 60)

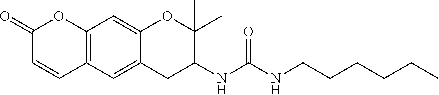 [SMILES: CCCCCCNC(=O)NC1Cc2cc3ccc(=O)oc3cc2OC1(C)C]

Compound 1 (150 mg, 0.61 mmol) and hexyl isocyanate (106 μL, 0.73 mmol) were reacted according to the method described in Preparation Example 3. The dichloromethane extract concentrate was purified and separated by silica gel column chromatography using a mixture of dichloromethane and methanol (v/v, 50:1), thereby affording Compound 60 as a white solid (133 mg, 59%).

Mp: 79° C.

¹H NMR (400 MHz, DMSO): δ 7.93 (d, J=9.5 Hz, 1H), 7.46 (s, 1H), 6.79 (s, 1H), 6.26 (d, J=9.5 Hz, 1H), 5.90 (d, J=9.1 Hz, 1H), 5.82 (t, J=5.6 Hz, 1H), 3.94 (dt, J=9.1, 5.5 Hz, 1H), 3.04 (dd, J=17.0, 5.0 Hz, 1H), 2.97 (ddd, J=8.9, 6.8, 2.9 Hz, 2H), 2.64 (dd, J=17.0, 5.8 Hz, 1H), 1.37-1.17 (m, 14H), 0.85 (td, J=6.8, 2.5 Hz, 3H).

¹³C NMR (101 MHz, DMSO): δ 160.75, 158.03, 156.74, 154.00, 144.60, 130.21, 117.87, 113.08, 113.00, 104.02, 79.04, 48.42, 31.46, 30.33, 29.30, 26.47, 25.81, 23.44, 22.54, 14.36.

MS (ESI): m/z [M+H]⁺ 373.2, found.

### Example 60: Preparation of 1-(8,8-dimethyl-2-oxo-7,8-dihydro-2H,6H-pyrano[3,2-g]chromen-7-yl)-3-nonylurea (Compound 61)

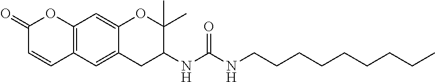 [SMILES: CCCCCCCCCNC(=O)NC1Cc2cc3ccc(=O)oc3cc2OC1(C)C]

Compound 1 (150 mg, 0.61 mmol) and nonyl isocyanate (142 μL, 0.73 mmol) were reacted according to the method described in Preparation Example 3. The dichloromethane extract concentrate was purified and separated by silica gel column chromatography using a mixture of dichloromethane and methanol (v/v, 50:1), thereby affording Compound 61 as a white solid (184 mg, 73%).

Mp: 122° C.

¹H NMR (400 MHz, DMSO): δ 7.93 (d, J=9.5 Hz, 1H), 7.46 (s, 1H), 6.79 (s, 1H), 6.26 (d, J=9.5 Hz, 1H), 5.89 (d, J=9.1 Hz, 1H), 5.81 (t, J=5.5 Hz, 1H), 3.94 (dt, J=9.2, 5.5 Hz, 1H), 3.04 (dd, J=17.0, 5.2 Hz, 1H), 2.99-2.92 (m, 2H), 2.64 (dd, J=16.9, 5.7 Hz, 1H), 1.35-1.30 (m, 2H), 1.29 (s, 3H), 1.24 (d, J=10.2 Hz, 15H), 0.85 (dd, J=7.7, 5.8 Hz, 3H).

¹³C NMR (101 MHz, DMSO): δ 160.75, 158.05, 156.74, 154.00, 144.60, 130.19, 117.88, 113.07, 113.00, 104.02, 79.04, 48.44, 31.74, 30.36, 29.45, 29.30, 29.23, 29.09, 26.80, 25.82, 23.40, 22.55, 14.41.

MS (ESI): m/z [M+H]⁺ 415.2, found.

### Preparation Example 4: Preparation of Decursin Amine Derivatives (X)

### 1) Preparation of Decursin Amine Derivative (X) by Reaction of Compound II with Benzylamine (IX)

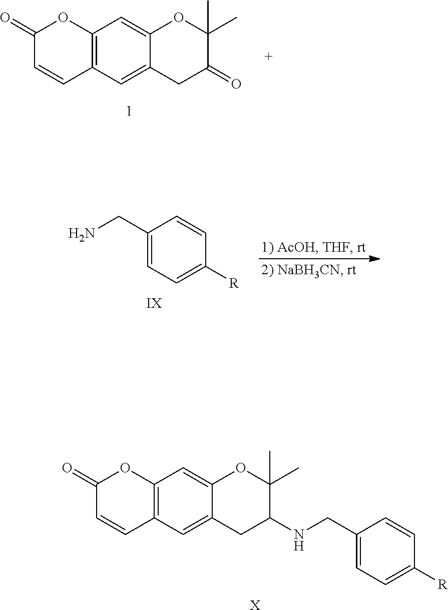

Amine derivatives of Formula (X) were prepared by reacting Compound 1, prepared in Preparation Example 1, with benzylamine derivatives of Formula (IX) according to the same method as that used for preparing Compound IV in 2) of Preparation Example 1.

The preparation of specific decursin amine derivatives is described in the following Examples.

### Example 61: Preparation of 7-(benzylamino)-8,8-dimethyl-7,8-dihydro-2H,6H-pyrano[3,2-g]chromen-2-one (Compound 62)

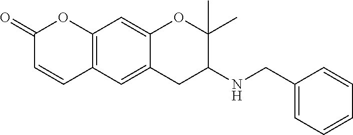 [SMILES: CC1(C)Oc2cc3oc(=O)ccc3cc2CC1NCc1ccccc1]

Compound II (100 mg, 0.41 mmol), prepared in Preparation Example 1, and benzylamine (68 μL, 0.62 mmol) were reacted according to the same method as that used for the preparation of Compound IV. The ethyl acetate extract concentrate was purified and separated by silica gel column chromatography using a mixture of hexane and ethyl acetate (v/v, 3:1), affording Compound 62 as a yellow semi-solid (110 mg, 80%).

¹H NMR (400 MHz, CDCl₃): δ 7.57 (d, J=9.5 Hz, 1H), 7.34-7.32 (m, 3H), 7.28-7.23 (m, 4H), 7.15 (s, 1H), 6.74 (s, 1H), 6.21 (d, J=9.5 Hz, 1H), 3.97 (d, J=13.4 Hz, 1H), 3.81 (d, J=13.4 Hz, 1H), 3.02 (dd, J=16.0, 4.7 Hz, 1H), 2.81 (dd, J=7.9, 4.8 Hz, 1H), 2.74-2.65 (m, 1H), 1.41 (s, 3H), 1.33 (s, 3H).

MS (ESI): m/z [M+H]⁺ 336.1, found.

### Example 62: Preparation of 8,8-dimethyl-7-((4-methylbenzyl)amino)-7,8-dihydro-2H,6H-pyrano[3,2-g]chromen-2-one (Compound 63)

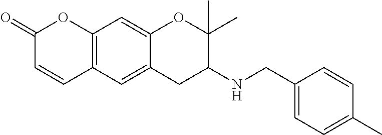 [SMILES: Cc1ccc(CNC2Cc3cc4ccc(=O)oc4cc3OC2(C)C)cc1]

Compound II (200 mg, 0.82 mmol), prepared in Preparation Example 1, and 4-methylbenzylamine (157 μL, 1.23 mmol) were reacted according to the same method as that used for the preparation of Compound IV. The ethyl acetate extract concentrate was purified and separated by silica gel column chromatography using a mixture of hexane and ethyl acetate (v/v, 3:1), affording Compound 63 as a white solid (190 mg, 66%).

Mp: 122° C.

¹H NMR (400 MHz, CDCl₃): δ 7.56 (d, J=9.5 Hz, 1H), 7.22 (d, J=7.8 Hz, 2H), 7.13 (d, J=8.3 Hz, 3H), 6.73 (s, 1H), 6.20 (d, J=9.4 Hz, 1H), 3.93 (d, J=13.2 Hz, 1H), 3.76 (d, J=13.2 Hz, 1H), 3.02 (dd, J=16.0, 4.5 Hz, 1H), 2.84-2.76 (m, 1H), 2.69 (dd, J=15.9, 8.0 Hz, 1H), 2.33 (s, 3H), 1.40 (s, 3H), 1.32 (s, 3H).

MS (ESI): m/z [M+H]⁺ 350.2, found.

### Example 63: Preparation of 7-((4-methoxybenzyl)amino)-8,8-dimethyl-7,8-dihydro-2H,6H-pyrano[3,2-g]chromen-2-one (Compound 64=Compound IV)

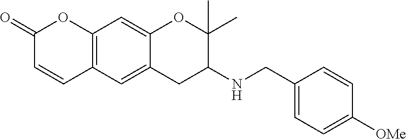 [SMILES: COc1ccc(CNC2Cc3cc4ccc(=O)oc4cc3OC2(C)C)cc1]

### Example 64: Preparation of 7-((4-fluorobenzyl)amino)-8,8-dimethyl-7,8-dihydro-2H,6H-pyrano[3,2-g]chromen-2-one (Compound 65)

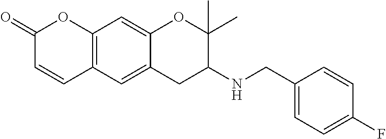 [SMILES: CC1(C)Oc2cc3oc(=O)ccc3cc2CC1NCc1ccc(F)cc1]

Compound II (200 mg, 0.82 mmol), prepared in Preparation Example 1, and 4-fluorobenzylamine (141 μL, 1.23 mmol) were reacted according to the same method as that used for the preparation of Compound IV. The ethyl acetate extract concentrate was purified and separated by silica gel column chromatography using a mixture of hexane and ethyl acetate (v/v, 3:1), affording Compound 65 as a yellow semi-solid (32 mg, 11%).

¹H NMR (400 MHz, CDCl₃): δ 7.57 (d, J=9.5 Hz, 1H), 7.30 (dd, J=8.4, 5.6 Hz, 2H), 7.15 (s, 1H), 7.00 (t, J=8.7 Hz, 2H), 6.73 (s, 1H), 6.20 (d, J=9.5 Hz, 1H), 3.94 (d, J=13.3 Hz, 1H), 3.77 (d, J=13.4 Hz, 1H), 3.03 (dd, J=15.9, 4.5 Hz, 1H), 2.78 (dd, J=7.7, 4.7 Hz, 1H), 2.70 (dd, J=15.9, 7.8 Hz, 1H), 1.40 (s, 3H), 1.33 (s, 3H).

MS (ESI): m/z [M+H]⁺ 354.1, found.

### Example 65: Preparation of 8,8-dimethyl-7-((4-nitrobenzyl)amino)-7,8-dihydro-2H,6H-pyrano[3,2-g]chromen-2-one (Compound 66)

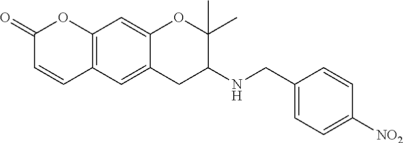 [SMILES: CC1(C)Oc2cc3oc(=O)ccc3cc2CC1NCc1ccc([N+](=O)[O-])cc1]

Compound II (200 mg, 0.82 mmol), prepared in Preparation Example 1, and 1-(4-nitrophenyl)methaneamine (149 μL, 1.23 mmol) were reacted according to the same method as that used for the preparation of Compound IV. The ethyl acetate extract concentrate was purified and separated by silica gel column chromatography using a mixture of hexane and ethyl acetate (v/v, 1:1), affording Compound 66 as a yellow semi-solid (17 mg, 5%).

¹H NMR (400 MHz, CDCl₃): δ 8.11 (d, J=8.7 Hz, 2H), 7.50 (d, J=9.5 Hz, 1H), 7.46 (d, J=8.7 Hz, 2H), 7.08 (s, 1H), 6.68 (s, 1H), 6.15 (d, J=9.5 Hz, 1H), 4.01 (d, J=14.6 Hz, 1H), 3.85 (d, J=14.6 Hz, 1H), 3.02-2.94 (m, 1H), 2.70 (ddd, J=22.6, 11.1, 5.6 Hz, 2H), 1.38 (s, 3H), 1.34 (s, 3H).

MS (ESI): m/z [M+H]⁺ 381.1, found.

### Example 66: Preparation of 8,8-dimethyl-7-(phenethylamino)-7,8-dihydro-2H,6H-pyrano[3,2-g]chromen-2-one (Compound 67)

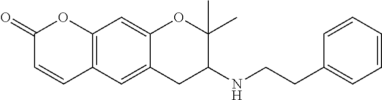 [SMILES: CC1(C)Oc2cc3oc(=O)ccc3cc2CC1NCCc1ccccc1]

Compound II (200 mg, 0.82 mmol), prepared in Preparation Example 1, and 2-phenylethylamine (151 μL, 1.23 mmol) were reacted according to the same method as that used for the preparation of Compound IV. The ethyl acetate extract concentrate was purified and separated by silica gel column chromatography using a mixture of hexane and ethyl acetate (v/v, 3:1), affording Compound 67 as a yellow semi-solid (134 mg, 47%).

¹H NMR (400 MHz, CDCl₃): δ 7.56 (d, J=9.5 Hz, 1H), 7.28 (dd, J=11.9, 4.8 Hz, 2H), 7.21 (dd, J=10.1, 4.4 Hz, 3H), 7.12 (s, 1H), 6.73 (s, 1H), 6.20 (d, J=9.5 Hz, 1H), 3.11-2.95 (m, 2H), 2.92-2.83 (m, 1H), 2.83-2.71 (m, 3H), 2.61 (dd, J=16.2, 8.4 Hz, 1H), 1.36 (s, 3H), 1.24 (s, 3H).

MS (ESI): m/z [M+H]⁺ 350.2, found.

### 2) Preparation of Decursin Amine Derivative (X) by Reaction of Compound 1 with Aldehyde

### Example 67: Preparation of 7-(cinnamylamino)-8,8-dimethyl-7,8-dihydro-2H,6H-pyrano[3,2-g]chromen-2-one (Compound 68)

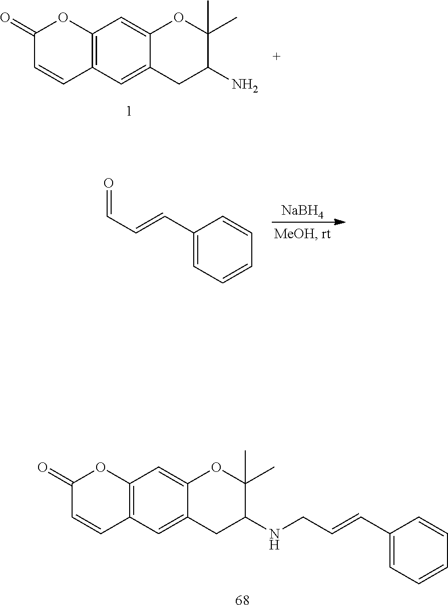

Compound 1 (55 mg, 0.22 mmol) was dissolved in methanol, and cinnamaldehyde (32 μL, 0.26 mmol) was added to the solution. The mixture was stirred at room temperature for 3 hours. After 3 hours, sodium borohydride (12 mg, 0.33 mmol) was added, and the reaction mixture was stirred at the same temperature for an additional 2 hours. Upon completion of the reaction, distilled water was added to quench the reaction, and the mixture was extracted with dichloromethane. The dichloromethane extract was dried over anhydrous sodium sulfate and concentrated, and the residue was purified and separated by silica gel column chromatography using a mixture of hexane and ethyl acetate (v/v, 1:2), thereby affording Compound 68 as a colorless semi-solid (34 mg, 43%).

¹H NMR (400 MHz, CDCl₃): δ 7.57 (d, J=9.4 Hz, 1H), 7.36 (d, J=7.1 Hz, 2H), 7.31 (t, J=7.3 Hz, 2H), 7.26-7.20 (m, 1H), 7.16 (s, 1H), 6.75 (s, 1H), 6.54 (d, J=15.8 Hz, 1H), 6.30-6.17 (m, 2H), 3.58 (dd, J=13.9, 5.8 Hz, 1H), 3.44 (dd, J=13.8, 6.3 Hz, 1H), 3.06 (dd, J=16.1, 4.4 Hz, 1H), 2.92-2.83 (m, 1H), 2.70 (dd, J=16.1, 7.9 Hz, 1H), 1.44 (s, 3H), 1.33 (s, 3H).

MS (ESI): m/z [M+H]⁺ 362.2, found.

### Experimental Example 1: Evaluation of Cytotoxicity

RAW 264.7 cells were purchased from the American Type Culture Collection (ATCC, Manassas, VA, USA). All cells were cultured in DMEM medium (Corning, NY, USA) supplemented with 10% fetal bovine serum (FBS; Sigma-Aldrich, St. Louis, MO, USA) and 1% penicillin-streptomycin (Sigma-Aldrich). Cells were maintained in an incubator (Thermo Fisher Scientific) at 37° C., 5% CO₂, and 95% humidity, and were subcultured every 2-3 days.

RAW 264.7 cells were seeded in 96-well plates at 2-5×10⁴ cells/well and incubated for 24 hours. After replacing the medium with fresh culture medium, the cells were treated with each of the test compounds prepared in the Examples at concentrations of 1, 10, or 100 μM, followed by incubation for an additional 24 hours. As a control, cells not treated with any test compound were incubated under the same conditions. After incubation, 10 μL of CCK-8 (Cell Count Kit-8, Dojindo, Korea) solution was added, and the plates were incubated for 2 hours in a CO₂ incubator (37° C., 5% CO₂). The absorbance was then measured at 450 nm, and cell viability was calculated as a percentage relative to the control group.

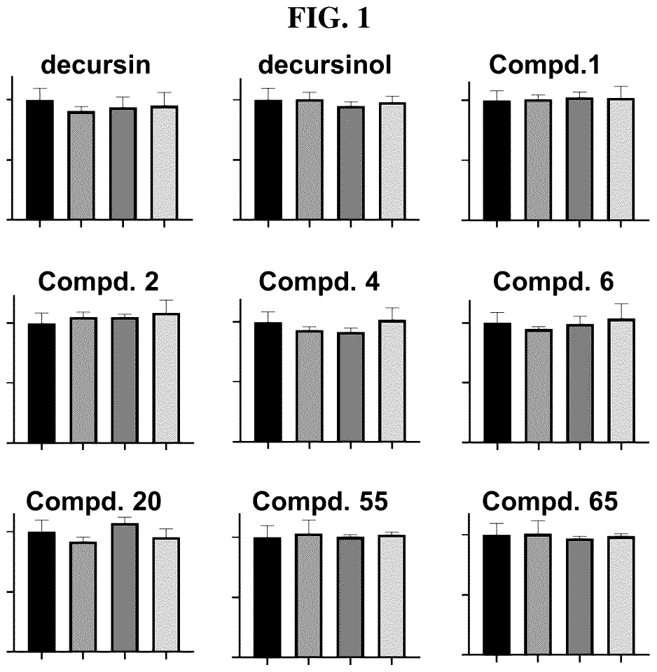

FIG. 1 shows the cell viability (%) results obtained after treatment with representative test compounds prepared in the Examples. In each graph of FIG. 1, the bars from left to right represent the control, 1 μM, 10 μM, and 100 μM treatment groups. The vertical axis indicates relative cell viability, with the control group set to 100% and 0% as the origin. All other test compounds prepared in the Examples that are not shown in FIG. 1 also exhibited no cytotoxicity within the above concentration range.

### Experimental Example 2: Evaluation of IL-6 Expression Inhibitory Activity in RAW 264.7 Cells

The anti-inflammatory activity of the decursin derivatives according to the present invention was evaluated by measuring the inhibition of lipopolysaccharide (LPS)-induced expression of interleukin-6 (IL-6).

RAW 264.7 cells were seeded in 48-well plates at 2.5×10⁵ cells/well and incubated for 24 hours. After confirming that the cells had adhered and reached 80-90% confluence, the medium was replaced with DMEM supplemented with 5% FBS. After 2 hours, 10 μL of LPS solution (5 g/mL) was added to each well, and the cells were incubated for 1 hour at 37° C. The test compounds prepared in the Examples were then added at various concentrations (e.g., 5, 10, and 20 μM), and incubation was continued for 18 hours. After treatment, the supernatant was collected and centrifuged at 13,000 rpm for 3 minutes at 4° C. The IL-6 level in the supernatant was measured using the BD OptEIA™ Mouse IL-6 ELISA Set (BD Biosciences, New Jersey, USA) according to the manufacturer's protocol.

For comparison, the anti-inflammatory activities of decursin and decursinol were also evaluated.

All data are expressed as mean±standard deviation. Statistical significance was assessed using one-way ANOVA and Tukey's multiple comparisons test with GraphPad Prism software. Compared with the group treated with LPS only (LPS-only group), statistical significance for groups treated with LPS plus a test compound was indicated as follows: * for 0.01<p<0.05, ** for 0.001<p<0.01, *** for 0.0001<p<0.001, and **** for p<0.0001.

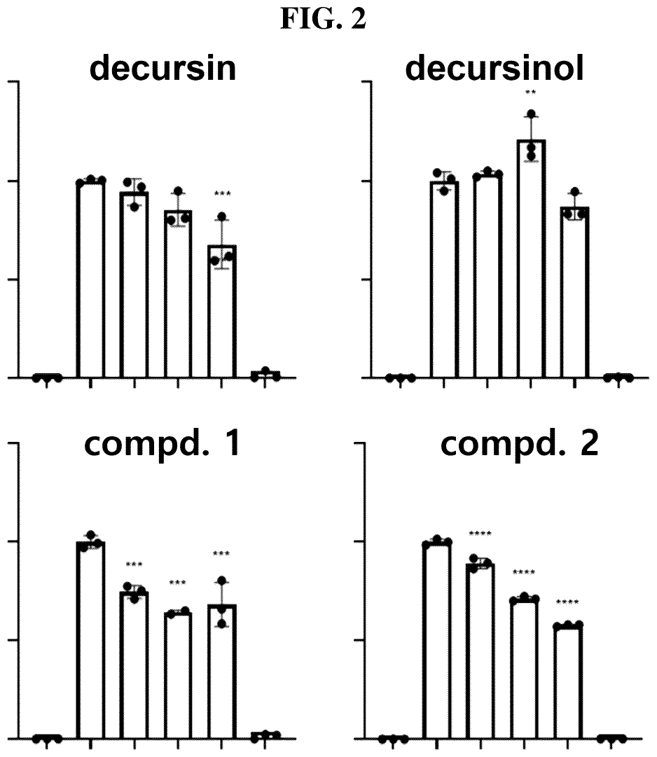

FIGS. 2 to 5 show graphs illustrating the results. In each graph in FIGS. 2 to 5, unless otherwise indicated, the bars from left to right represent the untreated group, the LPS-only group, the group treated with LPS plus a test compound at 5 μM, the group treated with LPS plus a test compound at 10 μM, the group treated with LPS plus a test compound at 20 μM, and the group treated with a test compound alone at 20 μM. The vertical axis indicates relative IL-6 expression, with the IL-6 level in the LPS-only group set to 100% and 0% as the origin. The numbers shown in each graph represent the compound numbers.

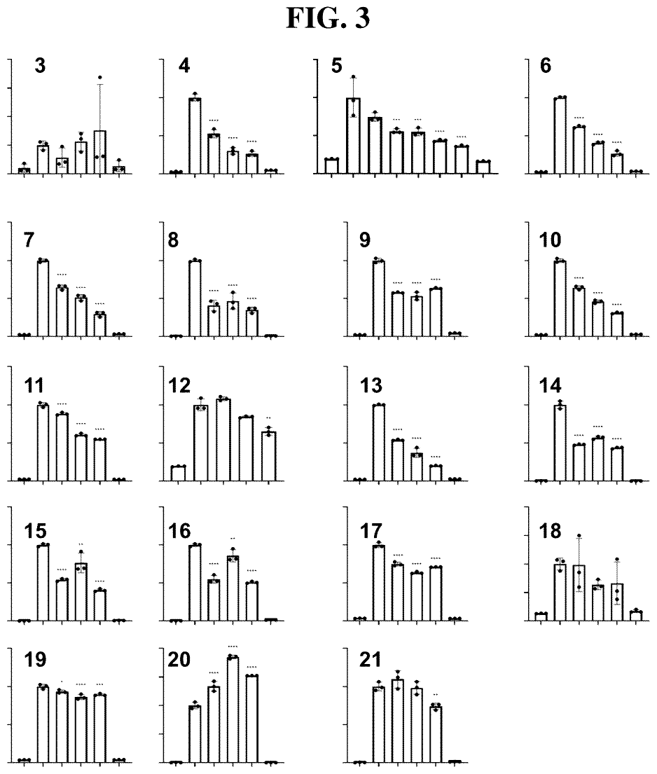

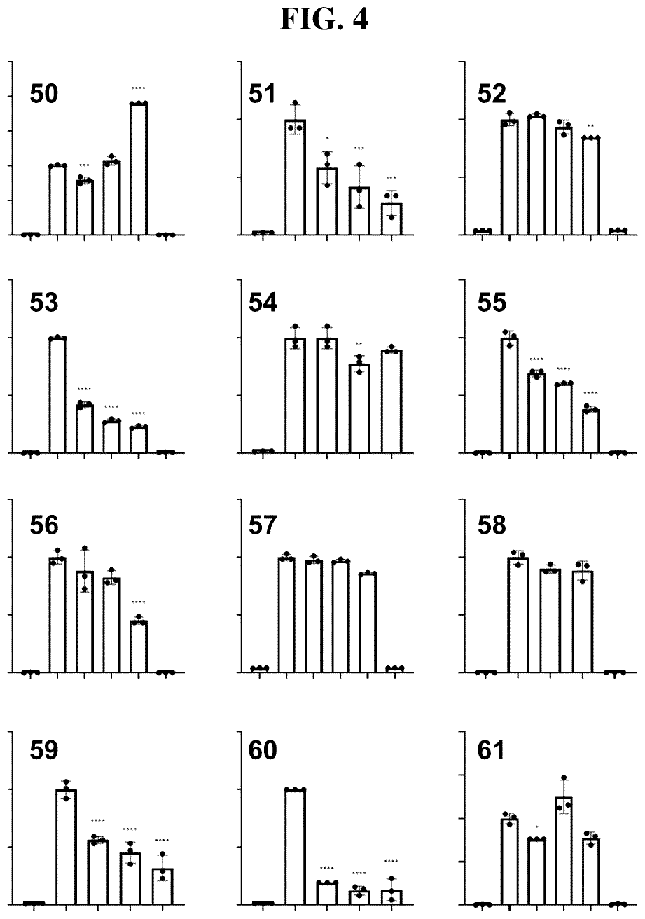

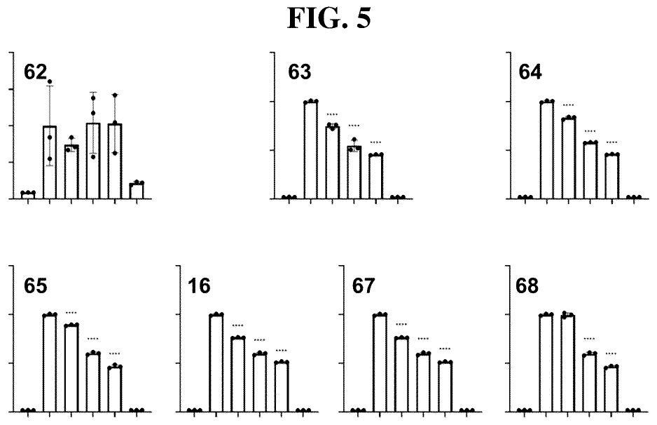

Specifically, FIG. 2 shows graphs illustrating the effects of decursinol, decursin, its amine analog decursinamine (Compound 1), and Compound 2 on IL-6 expression. FIG. 3 shows graphs illustrating the effects of representative decursin amide derivatives (Compounds 3-21) on IL-6 expression. FIG. 4 shows graphs illustrating the effects of representative decursin urea derivatives (Compounds 49-60) on IL-6 expression. FIG. 5 shows graphs illustrating the effects of representative decursin amine derivatives (Compounds 61-68) on IL-6 expression. In FIG. 3, for the graph of Compound 5, the third to seventh bars represent the groups treated with LPS plus a test compound at 0.25, 25, 50, 100, and 200 μM, respectively, and the rightmost bar represents the group treated with a test compound alone at 200 μM. In FIG. 4, the graphs for Compounds 51, 54, 58, 59, and 60 do not include the test compound-only treatment group.

As shown in FIG. 2, Compound 1 (decursinamine) and Compound 2, in which the ester group of decursin is replaced with an amide group, exhibited superior anti-inflammatory activity compared to decursinol. Overall, the amide, urea, and amine derivatives shown in FIGS. 3 to 5 demonstrated stronger anti-inflammatory activity than decursinol or decursin.

### Experimental Example 3: Evaluation of Effects on the Expression of Pro-Inflammatory Cytokines and Chemokines in Macrophages

Bone marrow cells were collected from the bone marrow of 8-week-old wild-type C57BL/6 mice. The collected bone marrow cells were suspended at a concentration of 1×10⁵/mL in culture medium [DMEM (Corning, Manassas, VA, USA) containing 4.5 g/L D-glucose, L-glutamine, and sodium pyruvate, supplemented with 10% fetal bovine serum (Gibco-BRL, Grand Island, NY, USA), penicillin G (100 IU/mL), and streptomycin (100 mg/mL)], and plated in 10-cm Petri dishes and incubated at 37° C. in 5% CO₂. After 2 hours, the cells were collected and centrifuged. Macrophage colony-stimulating factor (M-CSF) was added to the culture medium at a concentration of 25 ng/mL, and the cells were cultured for 3-4 days to induce differentiation into macrophages. Lymphocytes that had not differentiated into macrophages were removed by gently shaking the culture dishes and replacing the medium.

Macrophage cultures prepared as described above were treated with either LPS (100 ng/mL) alone or LPS (100 ng/mL) plus a test compound (20 μM) for 6 hours. After 6 hours, the medium was removed and the cells were washed with PBS. Total RNA was extracted using TRIzol reagent (Invitrogen, Carlsbad, CA, USA) according to the manufacturer's instructions and eluted in RNase-free water to a final volume of 40 μL. The extracted RNA was subjected to reverse transcription using 5× Reverse Transcriptase Pre-mix (Oligo dT) (ELPIS-BIOTECH, Daejeon, Korea) at 42° C. for 1 hour, and the reaction was then heated to 94° C. to terminate the reaction and obtain cDNA. The cDNA was diluted 1/10 with nuclease-free water, and qRT-PCR was performed to measure the expression levels of pro-inflammatory cytokines and chemokines. qRT-PCR was carried out using 2× QuantiNova SYBR Green PCR Master Mix (QIAGEN, Hilden, Germany), a primer solution prepared by mixing forward and reverse primers (10 pmol/μL each) at a 1:10 ratio, and diluted cDNA, by adding 10 μL each of the primer solution and the diluted cDNA. The reactions were performed on a Rotor-Gene Q 2plex system (QIAGEN) under the following conditions: denaturation at 95° C. for 20 s, annealing at 58° C. for 20 s, and elongation at 72° C. for 20 s, repeated for 40 cycles. Data were analyzed using the 2^{-ΔΔCt} threshold cycle method. The primer sequences used in this experiment are shown in Table 1.

TABLE 1

| | Sequences | SEQ ID NO |
|---|---|---|
| IL6 | 5′-TAC CAC TTC ACA AGT CGG-3′ | SEQ ID NO: 1 |
| | 5′-CTG CAA GTG CAT CAT CGT TGT TC-3′ | SEQ ID NO: 2 |
| Ccl2 | 5′-TGA CCC CAA GAA GGA ATG GG-3′ | SEQ ID NO: 3 |
| | 5′-ACC TTA GGG CAG ATG CAG TT-3′ | SEQ ID NO: 4 |
| Cxcl2 | 5′-CAT CCA CAG CTT GAG TGT GAC G-3′ | SEQ ID NO: 5 |
| | 5′-GGC TTC AGG GTC AAG GCA AAC T-3′ | SEQ ID NO: 6 |
| Cxcl5 | 5′-CCG CTG GCA TTT CTG TTG CTG T-3′ | SEQ ID NO: 7 |
| | 5′-CAG GGA TCA CCT CCA AAT TGA CG-3′ | SEQ ID NO: 8 |
| Cxcl10 | 5′-CCA AGT GCT GCC GTC ATT TT-3′ | SEQ ID NO: 9 |
| | 5′-CTC AAC ACG TGG GCA GGA TA-3′ | SEQ ID NO: 10 |
| Gapdh | 5′-AAG ATG GTG ATG GGC TTC CCG-3′ | SEQ ID NO: 11 |
| | 5′-TGG CAA AGT GGA GAT TGT TGC C-3′ | SEQ ID NO: 12 |

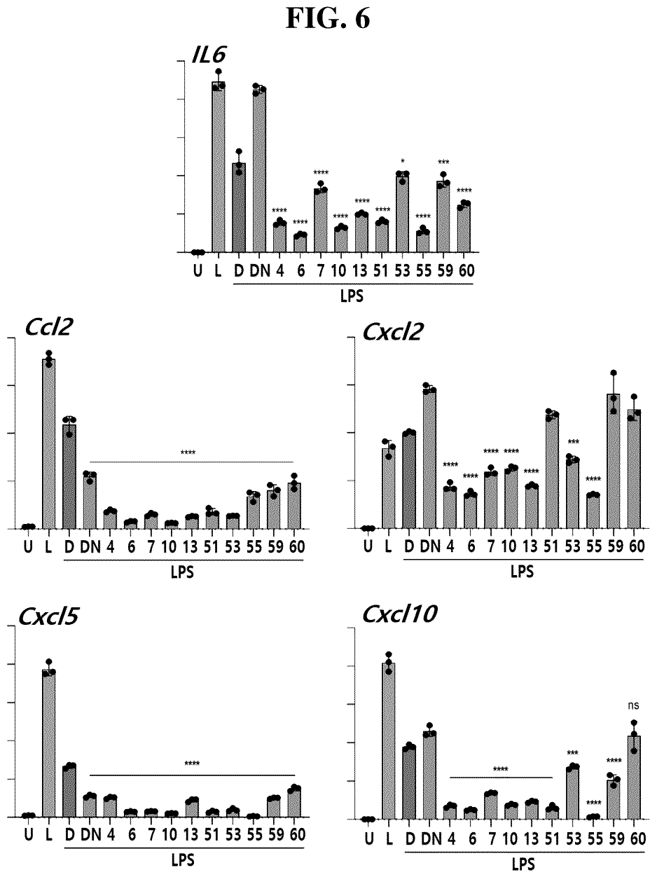

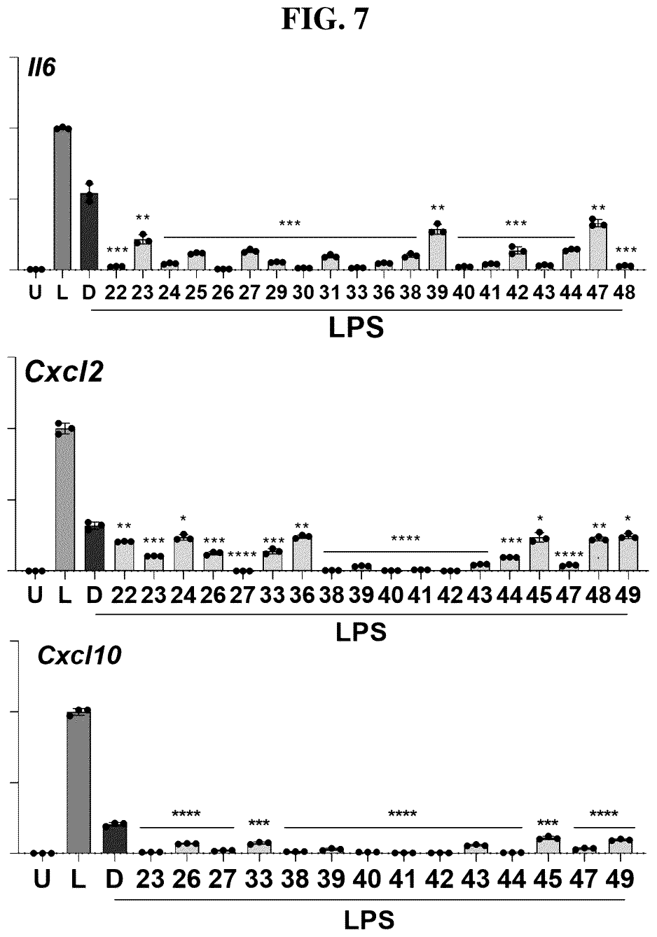

FIGS. 6 and 7 illustrate the effects of representative compounds on the expression of pro-inflammatory cytokines and chemokines induced by LPS stimulation. In each graph, “U” denotes the untreated group, “L” denotes the LPS-only group, “D” denotes decursin, “DN” denotes decursinol, and the numbers denote the compound numbers. Decursin, decursinol, and the representative compounds were all administered together with LPS. The mRNA expression levels were calculated as relative expression levels for each group, with the LPS-only group set at 100% and the untreated group set at 0%, and these relative values are shown on the vertical axis of the graphs.

As shown in FIGS. 6 and 7, the compounds of the present invention exhibited superior inhibitory effects on pro-inflammatory cytokines and chemokines compared with conventional decursin or decursinol.

1. A decursin derivative represented by Chemical Formula 1: 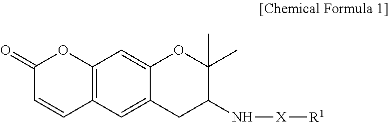
wherein X represents —C(═O)—, —C(═O)NH—, or a direct bond;
R¹ is —(CH═CH)—R², —(CH₂)ₙ—R³, or —(CH₂CH₂O)ₘ—R⁴;
n is an integer of 0 to 8, and m is an integer of 1 to 3;
R², R³, and R⁴ are each independently CH₃, C3-C6 cycloalkyl, 5- to 6-membered heterocycloalkyl, C6-C12 aryl, C4-C12 heteroaryl, 1,4-naphthoquinonyl, —CH═CH—R⁵, —S—R⁶, or —S(═O)—R⁷;
R⁵, R⁶, and R⁷ are each independently C6-C12 aryl, C4-C12 heteroaryl, or 1,4-naphthoquinonyl; and
R², R³, R⁴, R⁵, R⁶, and R⁷ may each independently be unsubstituted or substituted with a substituent.

2. The decursin derivative according to claim 1, wherein the substituent is selected from the group consisting of —F, —Cl, —Br, —CF₃, —OMe, —NO₂, -Me, —OH, —OAc, —SH, and —N(CH₃)₂, or a combination thereof.

3. The decursin derivative according to claim 1, wherein the 5- to 6-membered heterocycloalkyl is selected from the group consisting of pyrrolidine, piperidine, morpholine, thiomorpholine, and piperazine.

4. The decursin derivative according to claim 1, wherein the heteroaryl is selected from the group consisting of pyridine, pyrazine, furan, thiophene, and benzimidazole.

5. The decursin derivative according to claim 1, wherein the decursin derivative is in the form of a pharmaceutically acceptable salt.

6. A method for preparing a decursin derivative represented by Chemical Formula 1, the method comprising reacting decursin amine (1) with a carboxylic acid represented by R¹—COOH or an acyl halide or an ester thereof according to the following reaction scheme: 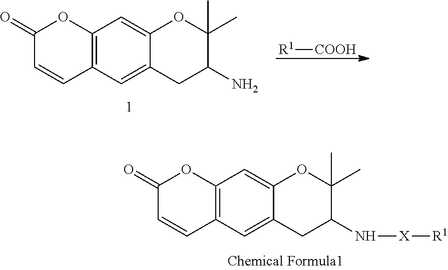
Wherein X represents —C(═O)—;
R¹ is —(CH═CH)—R², —(CH₂)ₙ—R³, or —(CH₂CH₂O)ₘ—R⁴;
n is an integer of 0 to 8, and m is an integer of 1 to 3;
R², R³, and R⁴ are each independently CH₃, C3-C6 cycloalkyl, 5- to 6-membered heterocycloalkyl, C6-C12 aryl, C4-C12 heteroaryl, 1,4-naphthoquinonyl, —CH═CH—R⁵, —S—R⁶, or —S(═O)—R⁷;
R⁵, R⁶, and R⁷ are each independently C6-C12 aryl, C4-C12 heteroaryl, or 1,4-naphthoquinonyl; and
R², R³, R⁴, R⁵, R⁶, and R⁷ may each independently be unsubstituted or substituted with a substituent.

7. A method for preparing a decursin derivative represented by Chemical Formula 1, the method comprising reacting decursin amine (1) with an isocyanate represented by R¹—NCO according to the following reaction scheme: 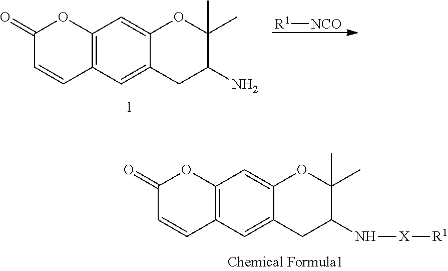
wherein X represents —C(═O)NH—;
R¹ is —(CH═CH)—R², —(CH₂)ₙ—R³, or —(CH₂CH₂O)ₘ—R⁴;
n is an integer of 0 to 8, and m is an integer of 1 to 3;
R², R³, and R⁴ are each independently CH₃, C3-C6 cycloalkyl, 5- to 6-membered heterocycloalkyl, C6-C12 aryl, C4-C12 heteroaryl, 1,4-naphthoquinonyl, —CH═CH—R⁵, —S—R⁶, or —S(═O)—R⁷;
R⁵, R⁶, and R⁷ are each independently C6-C12 aryl, C4-C12 heteroaryl, or 1,4-naphthoquinonyl; and
R², R³, R⁴, R⁵, R⁶, and R⁷ may each independently be unsubstituted or substituted with a substituent.

8. A method for preparing a decursin derivative represented by Chemical Formula 1, the method comprising:
(A) producing an imine or enamine intermediate by reacting decursin amine (1) with an aldehyde represented by R¹—CHO, or by coupling a compound (II) with an amine represented by R¹—NH₂; and
(B) reducing the imine or enamine intermediate to the corresponding amine,
according to the following reaction scheme: 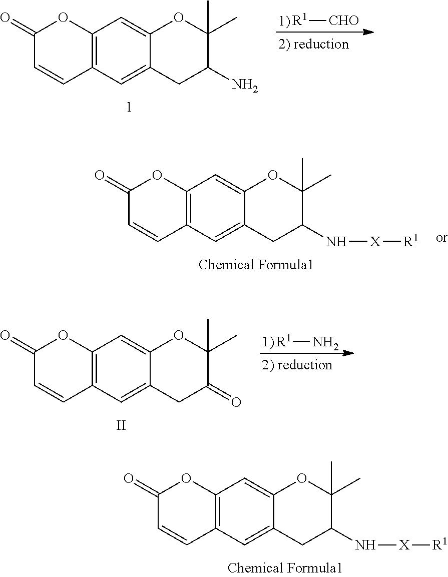
wherein X represents a direct bond;
R¹ is —(CH═CH)—R², —(CH₂)ₙ—R³, or —(CH₂CH₂O)ₘ—R⁴;
n is an integer of 0 to 8, and m is an integer of 1 to 3;
R², R³, and R⁴ are each independently CH₃, C3-C6 cycloalkyl, 5- to 6-membered heterocycloalkyl, C6-C12 aryl, C4-C12 heteroaryl, 1,4-naphthoquinonyl, —CH═CH—R⁵, —S—R⁶, or —S(═O)—R⁷;
R⁵, R⁶, and R⁷ are each independently C6-C12 aryl, C4-C12 heteroaryl, or 1,4-naphthoquinonyl; and
R², R³, R⁴, R⁵, R⁶, and R⁷ may each independently be unsubstituted or substituted with a substituent.

9. An anti-inflammatory pharmaceutical composition comprising a decursin derivative according to claim 1 or a pharmaceutically acceptable salt thereof.

10. A health functional food composition for the prevention or alleviation of inflammatory symptoms, comprising a decursin derivative according to claim 1 or a salt thereof.

11. A feed additive composition for the prevention or alleviation of inflammatory symptoms, comprising a decursin derivative according to claim 1 or a salt thereof.

12. A cosmetic composition for the prevention or alleviation of inflammatory symptoms, comprising a decursin derivative according to claim 1 or a salt thereof.
